(19) United States
(12) Patent Application Publication
(10) Pub. No.: US 2008/0194708 A1
(43) Pub. Date: Aug. 14, 2008

(54) Cationic Polymers as Thickeners for Aqueous and Alcoholic Compositions

(75) Inventors: Peter Hossel, Schifferstadt (DE); Klemens Mathauer, Heidelberg (DE); Claudia Wood, Weinheim (DE); Matthias Laubender, Schifferstadt (DE); Wolfgang Jahnel, Bellheim (DE); Ivette Garcia Castro, Ludwigshafen (DE); Volker Wendel, Frankfurt (DE); Karl Kolter, Limburgerhof (DE)

Correspondence Address: CONNOLLY BOVE LODGE & HUTZ LLP, 1875 EYE STREET, N.W., SUITE 1100, WASHINGTON, DC, 20036, US

(73) Assignee: BASF Aktiengesellschaft, Ludwigshafen (DE)

(21) Appl. No.: 11/909,034

(22) PCT Filed: Mar. 16, 2006
(86) PCT No.: PCT/EP2006/060802

§ 371 Date: Sep. 18, 2007

(30) Foreign Application Priority Data
Mar. 18, 2005 (DE) 102005013037.2
Aug. 18, 2005 (DE) 102005039537.6
Nov. 30, 2005 (EP) 05111530.1
Dec. 14, 2005 (EP) 05112165.5

(51) Int. Cl. A61K 8/81; C08L 39/04; C08L 33/24; C08F 220/52; C08F 226/06

(52) U.S. Cl. 514/772.5; 524/548; 524/555; 526/207; 514/772.4

(57) ABSTRACT

The present invention relates to the use of a water-soluble or water-dispersible crosslinked polymer obtainable by the polymerization of a mixture comprising 99.99 to 10% by weight of at least one α,β-ethylenically unsaturated compound having at least one cationogenic and/or cationic group per molecule, 0 to 90% by weight of at least one monoethylenically unsaturated compound containing amide groups which differs from a), and 0.01 to 5% by weight of a crosslinking agent for modifying the rheology of aqueous compositions.

The present invention relates to the use of a water-soluble or water-dispersible crosslinked polymer obtainable by the polymerization of a mixture comprising 99.99 to 10% by weight of at least one α,β-ethylenically unsaturated compound having at least one cationogenic and/or cationic group per molecule, 0 to 90% by weight of at least one monoethylenically unsaturated compound containing amide groups which differs from a), and 0.01 to 5% by weight of a crosslinking agent for modifying the rheology of aqueous compositions.

Polymers have many uses in hair cosmetics. Their purpose in hair cosmetics consists in influencing the properties of the hair and especially to strengthen the hair, improve the ease of combing and confer a pleasant feel.

Conditioners are thus used to improve the ease of dry and wet combing, improve the feel, luster and appearance and confer antistatic properties on the hair. It is preferable to use water-soluble polymers with polar and often cationic functional groups; these have a greater affinity for the surface of the hair, which is negatively charged for structural reasons. The structure and mode of action of various hair treatment polymers are described in Cosmetic & Toiletries 103 (1988) 23. Examples of commercially available conditioning polymers are cationic hydroxyethyl cellulose, cationic polymers based on N-vinylpyrrolidone, e.g. copolymers of N-vinylpyrrolidone and quaternized N-vinylimidazole, acrylamide and diallyldimethylammonium chloride, or silicones.

Hair styles are strengthened using vinyllactam homopolymers and copolymers and polymers containing carboxylate groups. The requirements of hair strengthening resins are e.g. a good strengthening action at high humidity, elasticity, ease of rinsing out of the hair, compatibility in the formulation and a pleasant feel of the hair.

It is often difficult to combine different properties such as a good strengthening action, a pleasant feel of the hair and a simultaneous thickening action of the polymers in cosmetic formulations for the hair.

This is particularly important in gel formulations. Furthermore, conventional strengthening polymers frequently exhibit incompatibilities with thickening polymers, giving rise to turbidity and precipitation in the cosmetic formulations. Classical thickeners, which consist of either crosslinked polyacrylic acid (Carbopol) or copolymers, have the disadvantage that, because of the crosslinking, they do not form films suitable for strengthening the hair. They are concerned with the texture of the gel, but are no longer required after the gel has dried on the hair, so they potentially interfere with the application properties of the formulation (strengthening action, moisture sensitivity).

### STATE OF THE ART

DD-117 326 (Jeschek, H.) describes a process for increasing the sensitivity and improving the stability of photographic gelatin/silver halide emulsion layers by means of polyethylene glycol derivatives, wherein graft polymers of polyethylene glycol of average molecular weight 1500-6000 and vinyl compounds comprising quaternary nitrogen are added to the gelatin/silver halide emulsions and/or their developers. A use for modifying the rheology of aqueous compositions is not described.

WO 03/080001 (BASF) relates to cationic graft polymers obtainable by the polymerization of quaternized cationic monomers polymerizable by free radical polymerization, and optionally other monomers copolymerizable by free radical polymerization, in the presence of a polyether-comprising compound, with the proviso that the reaction takes place in the presence of less than 20% by weight of water in the total reaction mixture.

The use for modifying the rheology of aqueous compositions is not described.

WO 03/068834 describes the use of graft copolymers as a constituent of cosmetic products, the graft copolymers being formed by grafting monoethylenically unsaturated, open-chain monomers comprising N-vinylamide units onto a polymeric base grafting material.

WO 03/106522 (BASF) describes the use of polyoxyalkylene-substituted alkylenediamines in cosmetic formulations. Modified polyoxyalkylene-substituted alkylenediamines, especially cationic polymers, and processes for their preparation are also included. The polymers comprise at least 40% by weight of polyoxyalkylene-substituted alkylenediamines.

WO 04/100910 (BASF) describes cosmetic products containing at least one polymer obtainable by the free radical polymerization of α,β-ethylenically unsaturated compounds, each of which comprises at least one nitrogen-containing heterocycle, in the presence of a polymeric base grafting material. Vinylpyrrolidone/vinylimidazole copolymers prepared in the presence of polyethylene glycol are described in particular. Water is used as solvent in the polymerization and the polymers are neither crosslinked nor used for modifying the rheology.

WO 03/042262 (BASF) relates to graft polymers containing a polymeric base grafting material (A) which does not have monoethylenically unsaturated units, and polymeric side chains (B) which are formed of copolymers of two different monoethylenically unsaturated monomers (B1) and (B2), each of which comprises at least one nitrogen-containing heterocycle, the proportion of side chains (B) in the total polymer being 35 to 55% by weight. Water is used as solvent in the polymerization and the polymers are not crosslinked. The preparation of these graft polymers and their use as color transfer inhibitors in detergents are further described, but their use for modifying the rheology is not disclosed.

The subject matter of WO 03/042264 (BASF) differs from that of WO 03/042262 in that the proportion of side chains (B) in the total polymer is more than 55% by weight.

WO 93/22380 (ISP) discloses hydrogels, adhesives and coatings comprising crosslinked copolymers of 80-99% by weight of N-vinylpyrrolidone and 1 to 20% by weight of N-vinylimidazole or 4-vinylpyridine which have been prepared by solution polymerization in water.

DE 198 33 287 (BASF) describes the preparation of polymers of (a1) 5 to 99.99% by weight of a monomer polymerizable by free radical polymerization and comprising a quaternized or quaternizable nitrogen atom, or mixtures of such monomers, (a2) 5 to 95% by weight of an N-vinyllactam, (b) 0.01 to 20% by weight of a monomer having a crosslinking action and containing at least two ethylenically unsaturated groups, and (c) 0 to 50% by weight of another monomer polymerizable by free radical polymerization, in supercritical carbon dioxide as inert diluent, at temperatures of between 31° C. and 150° C. and pressures above 73 bar, with thorough mixing.

DE 100 23 245 (BASF) describes cosmetic products for the hair containing (i) polymer obtainable by the free radical-initiated polymerization of (a) 1 to 100% by weight, preferably 2 to 95% by weight and particularly preferably 10 to 70% by weight of at least one cationic monomer selected from N-vinylimidazoles and diallylamines, optionally in partially or completely quaternized form, (b) 0 to 99% by weight, preferably 5 to 98% by weight and particularly preferably 30 to 90% by weight of at least one water-soluble monomer differing from (a), and (c) 0 to 50% by weight, preferably 0 to 40% by weight and particularly preferably 0 to 30% by weight of at least one other monomer copolymerizable by free radical polymerization and differing from (a) and (b), followed by partial or complete quaternization or protonation of the polymer if an unquaternized or only partially quaternized monomer is used as monomer (a), and (ii) straightening agent. The use of such polymers for modifying the rheology of aqueous compositions is not described

DE 197 31 907 (BASF) describes crosslinked cationic polymers of vinylimidazole and N-vinylpyrrolidone which are prepared by gel polymerization, reverse suspension polymerization or inverse emulsion polymerization in aqueous solvent mixtures.

DE 102 41 296 (BASF) describes a process for the preparation of aqueous dispersions of water-soluble or water-swellable, crosslinked cationic polymers based on monoethylenically unsaturated monomers comprising a quaternized or quaternizable nitrogen atom, by free radical polymerization in an aqueous salt solution in the presence of a protective colloid, and their use in cosmetic formulations for the hair. The polymers are prepared by water-in-water emulsion polymerization.

U.S. Pat. No. 4,806,345 describes crosslinked cationic thickeners for cosmetic formulations, consisting of quaternized dimethylaminoethyl methacrylate and acrylamide.

WO 93/25595 describes crosslinked cationic copolymers based on quaternized dialkylaminoalkyl acrylates or dialkylaminoalkylacrylamides. The proposed use for these crosslinked copolymers is as thickeners in cosmetic formulations.

DE A 32 09 224 describes the preparation of crosslinked polymers based on N-vinylpyrrolidone and (quaternized) N-vinylimidazole. These polymers are claimed for use as adsorbents and ion exchangers.

WO 96/37525 describes the preparation of crosslinked copolymers of N-vinyl-pyrrolidone and quaternized vinylimidazoles, inter alia, in the presence of polymerization regulators, and their use especially in detergents.

U.S. Pat. No. 4,058,491 discloses crosslinked cationic hydrogels of N-vinylimidazole or N-vinylpyrrolidone and a quaternized basic acrylate and other comonomers. These gels are proposed for the complexation and controlled release of anionic active substances.

DE A 42 13 971 describes copolymers of an unsaturated carboxylic acid, quaternized vinylimidazole and optionally other monomers and a crosslinking agent. The polymers are proposed as thickeners and dispersants.

WO 97/35544 describes the use of crosslinked cationic polymers with dialkylaminoalkyl(meth)acrylates or dialkylaminoalkyl(meth)acrylamides in shampoo compositions.

EP A 0 893 117 and EP 1 064 924 describe the use of high-molecular crosslinked cationic polymers as solution polymers having a good conditioning effect in shampoos.

DE A 19731907 describes the use of crosslinked cationic copolymers comprising N-vinylimidazoles in cosmetic formulations for the hair.

### OBJECT OF THE INVENTION

One object of the present invention was to find polymers which are suitable for cosmetic applications and, for example in the field of hair cosmetics, have good application properties such as a pleasant feel and at the same time a good conditioning effect or a good strengthening action and at the same time a thickening property. The solubility of the polymers to give a clear solution in conventional cosmetic formulations for the hair was of particular interest here.

A further object was to provide polymers which satisfy both cosmetic requirements for the hair (strengthening action, curl retention) and application requirements (thickening properties, solubility to give a clear solution).

A further object of the present invention was to find polymers which combine a thickening action with film-forming properties so that these polymers can also be used as strengthening polymers in e.g. hair gel formulations.

There was particular interest in polymers having thickening properties even without the addition of other, conventional thickeners (e.g. high-molecular polyacrylic acids of the Carbopol™ type).

A further object of the present invention was to provide polymers which assure a high salt loading stability of aqueous compositions containing them. These polymers should also be compatible with cationic polymers in the pH range from 5 to 8.

In particular, water-soluble or water-dispersible polymers based on cost-effective starting materials should be provided which, compared with the state of the art, have better properties as regards modification of the rheology of water-containing compositions, especially in the field of cosmetic formulations (for the hair). The use of even small amounts of polymers should allow an effective modification of the rheology.

Furthermore, this effective modification of rheology should also be facilitated over the widest possible pH range. The polymers should further be accessible by a cost-effective process which moreover requires comparatively small product work-up procedures. Furthermore, the solubility of the polymers to give a clear solution in conventional cosmetic formulations was of particular interest, and the skin or hair treated with the compositions and products comprising these polymers should have good sensory properties.

In the field of cosmetic formulations for the hair, especially emulsions, there is a need for polymeric thickeners which have a good thickening action even at high salt concentrations and pH values of 5 to 8.

One object was thus to provide polymers which satisfy both cosmetic requirements (for the hair) and application requirements (thickening properties, solubility to give a clear solution).

The aforesaid objects have been achieved by the use of a water-soluble or water-dispersible polymer obtainable by the polymerization of a mixture comprising
a) 99.99 to 10% by weight of at least one α,β-ethylenically unsaturated compound having at least one cationogenic and/or cationic group per molecule,
b) 0 to 90% by weight of at least one monoethylenically unsaturated compound containing amide groups which differs from a),
c) 0.01 to 5% by weight of a crosslinking agent,
d) 0 to 15% by weight of at least one monoethylenically unsaturated compound d1) comprising at least one group selected from the group comprising optionally substituted C5-C30-alkyl, C5-C30-alkenyl, C5-C8-cycloalkyl, aryl, arylalkyl and heteroaryl,
   and/or a reactive precursor d2), and
e) 0 to 30% by weight of other monoethylenically unsaturated compounds differing from a) to d),
   in the presence of
f) 0 to 70% by weight, based on the sum of components a) to e), of a polyether-containing compound,
   with the proviso that the amounts of components a) to e) add up to 100% by weight,
   the polymerization being carried out in the presence of less than 69% by weight of cyclohexane and less than 12% by weight of water, based on the total amount of all the components present during the polymerization, and in the absence of supercritical carbon dioxide,
   for modifying the rheology of aqueous compositions.

One preferred embodiment of the invention is the use of the abovementioned polymers wherein the α,β-ethylenically unsaturated compound a) is selected from
ai) esters of α,β-ethylenically unsaturated monocarboxylic and dicarboxylic acids with amino alcohols which can be monoalkylated or dialkylated on the amine nitrogen,
aii) amides of α,β-ethylenically unsaturated monocarboxylic and dicarboxylic acids with diamines which have at least one primary or secondary amino group,
aiii) N,N-diallylamines,
aiv) vinyl- and allyl-substituted nitrogen heterocycles,
av) vinyl- and allyl-substituted heteroaromatic compounds and
avi) mixtures thereof.

Within the framework of the present invention the term alkyl comprises linear and branched alkyl groups. Examples of suitable short-chain alkyl groups are linear or branched C1-C7-alkyl, preferably C1-C6-alkyl and particularly preferably C1-C4-alkyl groups. These include especially methyl, ethyl, propyl, isopropyl, n-butyl, 2-butyl, sec-butyl, tert-butyl, n-pentyl, 2-pentyl, 2-methylbutyl, 3-methylbutyl, 1,2-dimethyl-propyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-tri-methylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl, etc.

Suitable longer-chain C8-C30-alkyl or C8-C30-alkenyl groups are linear and branched alkyl or alkenyl groups. Preferably, these are predominantly linear alkyl radicals such as those also occurring in natural or synthetic fatty acids and fatty alcohols, as well as oxo alcohols, which may additionally be mono-, di- or polyunsaturated. These include e.g. n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene), etc.

Cycloalkyl is preferably C5-C8-cycloalkyl such as cyclopentyl, cyclohexyl, cycloheptyl or cyclooctyl.

Within the framework of the present invention the term heterocycloalkyl comprises saturated cycloaliphatic groups having in general 4 to 7 and preferably 5 or 6 ring atoms, in which 1 or 2 of the ring carbon atoms have been replaced by heteroatoms selected from the elements oxygen, nitrogen and sulfur, and which can optionally be substituted, it being possible, in the case of substitution, for these heterocycloaliphatic groups to carry 1, 2 or 3, preferably 1 or 2 and particularly preferably 1 substituent selected from alkyl, aryl, COOR, COO⁻M⁺ and NE¹E², alkyl being preferred. Examples of such heterocycloaliphatic groups which may be mentioned are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, tetrahydrofuranyl, tetrahydropyranyl and dioxanyl.

Aryl comprises unsubstituted and substituted aryl groups and is preferably phenyl, tolyl, xylyl, mesityl, naphthyl, fluorenyl, anthracenyl, phenanthrenyl or naphthacenyl, especially phenyl, tolyl, xylyl or mesityl.

Substituted aryl radicals have preferably 1, 2, 3, 4 or 5 and especially 1, 2 or 3 substituents selected from alkyl, alkoxy, carboxyl, carboxylate, trifluoromethyl, —SO₃H, sulfonate, NE¹E², alkylene-NE¹E², nitro, cyano and halogen.

Heteroaryl is preferably pyrrolyl, pyrazolyl, imidazolyl, indolyl, carbazolyl, pyridyl, quinolinyl, acridinyl, pyridazinyl, pyrimidinyl or pyrazinyl.

Arylalkyl represents groups containing both alkyl and aryl radicals, said arylalkyl groups being linked either via the aryl radical or via the alkyl radical to the compound carrying them.

Component a)

In general, examples of monomers a) which can be used to prepare the polymers suitable for the use according to the invention are the compounds referred to as “direct precursors a2” on p. 18, line 27 to p. 22, line 38 of WO 03/080001. Said publication is incorporated here to the full extent by way of reference.

Suitable cationogenic monomers a) are ai) the esters of α,β-ethylenically unsaturated monocarboxylic and dicarboxylic acids with amino alcohols. Preferred amino alcohols are C₂-C₁₂-amino alcohols which are C₁-C₈-monoalkylated or -dialkylated on the amine nitrogen. Examples of suitable acid components of these esters are acrylic acid, methacrylic acid, fumaric acid, maleic acid, itaconic acid, crotonic acid, maleic anhydride, monobutyl maleate and mixtures thereof.

Other suitable monomers a) are aii) the amides of the aforementioned α,β-ethylenically unsaturated monocarboxylic and dicarboxylic acids with diamines having at least one primary or secondary amino group. Diamines having one tertiary and one primary or secondary amino group are preferred.

Preferred acid components are acrylic acid, methacrylic acid and mixtures thereof.

Preferred esters ai) or amides aii) of α,β-ethylenically unsaturated monocarboxylic and dicarboxylic acids with amino alcohols or diamines, respectively, are thus aminoalkyl(meth)acrylates and aminoalkyl(meth)acrylamides of general formula I:

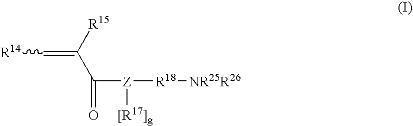

in which
R¹⁴ and R¹⁵ independently of one another are selected from the group comprising hydrogen, linear or branched C₁-C₈-alkyl, methoxy, ethoxy, 2-hydroxy-ethoxy, 2-methoxyethoxy and 2-ethoxyethyl, preference being given to hydrogen, methyl or ethyl,
R¹⁷ is hydrogen or methyl,
R¹⁸ is alkylene or hydroxyalkylene having 1 to 24 C atoms and optionally substituted by alkyl, preferably C₂H₄, C₃H₆, C₄H₈ or CH₂—CH(OH)—CH₂,
g is 0 or 1,
Z is nitrogen for g=1 or oxygen for g=0, and
R²⁵ and R²⁶ independently of one another are each selected from the group comprising hydrogen, linear or branched C₁-C₄₀-alkyl, formyl, linear or branched C₁-C₁₀-acyl, N,N-dimethylaminoethyl, 2-hydroxyethyl, 2-methoxyethyl, 2-ethoxyethyl, hydroxypropyl, methoxypropyl, ethoxy-propyl and benzyl, preference being given to hydrogen, methyl, ethyl, n-propyl and benzyl.

The following are particularly preferred as component ai):

N-methylaminoethyl(meth)acrylate, N-ethylaminoethyl(meth)acrylate, N-(n-propyl)aminoethyl(meth)acrylate, N-(n-butyl)aminoethyl(meth)acrylate, N-(tert-butyl)aminoethyl(meth)acrylate, N,N-dimethylaminomethyl(meth)acrylate, N,N-dimethylaminoethyl(meth)acrylate, N,N-diethylaminoethyl(meth)acrylate, N,N-dimethylaminopropyl(meth)acrylate, N,N-diethylaminopropyl(meth)acrylate and N,N-dimethylaminocyclohexyl(meth)acrylate. N-(tert-butyl)aminoethyl acrylate and N-(tert-butyl)aminoethyl methacrylate are used in particular as component ai).

The amides can be unsubstituted, monosubstituted by N-alkyl or N-alkylamino, disubstituted by N,N-dialkyl or disubstituted by N,N-dialkylamino, the alkyl or alkylamino groups being derived from linear C₁-C₄₀, branched C₃-C₄₀ or carbocyclic C₃-C₄₀ units.

Preferred components aii) are

N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide, N-[4-(dimethylamino)cyclohexyl]methacrylamide, N-[8-(dimethylamino)octyl]-methacrylamide, N-[12-(dimethylamino)dodecyl]methacrylamide, N-[3-(diethyl-amino)propyl]methacrylamide and N-[3-(diethylamino)propyl]acrylamide.

The cationogenic monomer a) can also be selected from aiii) N,N-diallylamines of general formula II:

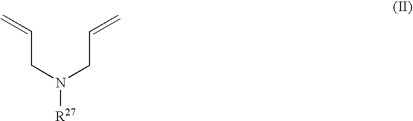

in which R²⁷ is hydrogen or C₁- to C₂₄-alkyl. N,N-diallylamine and especially N,N-diallyl-N-methylamine are particularly preferred. N,N-diallyl-N-methylamine commercially available in a quaternized form, e.g. under the name DADMAC (diallyldimethylammonium chloride), is particularly preferred.

Other particularly preferred cationogenic monomers a) are aiv) vinyl- and allyl-substituted nitrogen heterocycles such as N-vinylimidazole and N-vinylimidazole derivatives, e.g. N-vinyl-2-methylimidazole, and vinyl- and allyl-substituted heteroaromatic compounds such as 2- and 4-vinylpyridine and 2- and 4-allylpyridine.

Very particularly preferred N-vinylimidazoles are those of general formula (III):

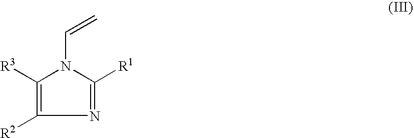

in which R¹ to R³ are hydrogen, C₁-C₄-alkyl or phenyl.

Examples of compounds of general formula (III) can be found in Table 1 below:

TABLE 1

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

The most preferred component a) is aiv) N-vinylimidazole, i.e. the compound of formula III in which all the radicals R¹ to R³ are hydrogen.

The conversion of the compounds a) to quaternary compounds can take place during or, preferably, after the reaction. In the case of a subsequent conversion, the intermediate polymer can be isolated and purified first or converted directly. The conversion can be total or partial. Preferably at least 10%, particularly preferably at least 20% and very particularly preferably at least 30% of the incorporated monomers a) are converted to the corresponding quaternary form. The degree of conversion to quaternary compounds is preferably inversely proportional to the solubility of the monomer a) in water.

Preferably, the monomers a) are used for the polymerization in predominantly cationogenic form, i.e. more than 70, preferably more than 90, particularly preferably more than 95 and very particularly preferably more than 99 mol % cationogenic, i.e. not in quaternized or protonated form, and only converted to the cationic or protonated form by quaternization during or, particularly preferably, after the polymerization.

Protonation/Quaternization

In one preferred embodiment of the invention the polymer is partially or completely protonated or quaternized only during or, particularly preferably, after the polymerization, because the component a) used for the polymerization is preferably a monomer that is only partially quaternized or protonated, if at all.

The monomers a) can either be used in protonated or quaternized form or, preferably, polymerized in unquaternized or unprotonated form, the copolymer obtained in the latter case being either quaternized or protonated during or, preferably, after the polymerization for the use according to the invention.

In the case where the monomers are used in quaternized form, they can be used either as the dried substance, or in the form of concentrated solutions in solvents suitable for the monomers, e.g. in polar solvents such as water, methanol, ethanol or acetone, or in the other components a) to f) provided these are suitable as solvents, or in electrolyte solutions.

Examples of compounds suitable for the protonation are mineral acids such as HCl and H₂SO₄, monocarboxylic acids, e.g. formic acid and acetic acid, dicarboxylic acids and polyfunctional carboxylic acids, e.g. oxalic acid and citric acid, and any other proton-donating compounds and substances that are capable of protonating the appropriate nitrogen atom. Water-soluble acids are particularly suitable for the protonation.

Possible organic acids which may be mentioned are optionally substituted monobasic and polybasic aliphatic and aromatic carboxylic acids, optionally substituted monobasic and polybasic aliphatic and aromatic sulfonic acids or optionally substituted monobasic and polybasic aliphatic and aromatic phosphonic acids.

Preferred organic acids are hydroxycarboxylic acids such as glycolic acid, lactic acid, tartaric acid and citric acid, lactic acid being particularly preferred.

Preferred inorganic acids which may be mentioned are phosphoric acid, phosphorous acid, sulfuric acid, sulfurous acid and hydrochloric acid, phosphoric acid being particularly preferred.

The polymer is protonated either directly after the polymerization or only when the cosmetic product is formulated, during which the pH is normally adjusted to a physiologically acceptable value.

Protonation is understood as meaning that at least some of the protonatable groups of the polymer, preferably at least 20, preferably more than 50, particularly preferably more than 70 and very particularly preferably more than 90 mol %, are protonated, resulting in an overall cationic charge on the polymer.

Examples of suitable reagents for quaternizing the compounds a) are alkyl halides having 1 to 24 C atoms in the alkyl group, e.g. methyl chloride, methyl bromide, methyl iodide, ethyl chloride, ethyl bromide, propyl chloride, hexyl chloride, dodecyl chloride, lauryl chloride, propyl bromide, hexyl bromide, dodecyl bromide and lauryl bromide, and benzyl halides, especially benzyl chloride and benzyl bromide. The preferred quaternizing agent is methyl chloride. Quaternization with long-chain alkyl radicals is preferably carried out with the corresponding alkyl bromides such as hexyl bromide, dodecyl bromide or lauryl bromide.

Other suitable quaternizing agents are dialkyl sulfates, especially dimethyl sulfate or diethyl sulfate.

The quaternization of the basic monomers a) can also be carried out with alkylene oxides such as ethylene oxide or propylene oxide, in the presence of acids.

Preferred quaternizing agents are methyl chloride, dimethyl sulfate or diethyl sulfate, methyl chloride being particularly preferred.

The quaternization of the monomers or polymers with one of said quaternizing agents can be effected by generally known methods.

According to the invention, the polymers can be used for modifying the rheology of aqueous compositions in the pH range from 1 to 12 and preferably from 2 to 10.

In the pH range from 1 to 5 it is advantageous if the quaternizable groups of the polymers are less than 20%, preferably less than 10% and particularly preferably less than 1% quaternized.

In the pH range from 6 to 10 it is advantageous if the quaternizable groups of the polymers are at least 10% and preferably at least 20% quaternized and at most 99% and preferably at most 90% quaternized.

Between pH 5 and pH 6 the polymers can advantageously be either partially quaternized or unquaternized, depending on their quantitative and/or qualitative monomer composition.

Routine experiments enable those skilled in the art to discover whether a particular polymer is advantageously quaternized or unquaternized in this pH range. Further routine experiments also make it possible to determine the degree of quaternization (=quotient of the amount of quaternized groups and the sum of the amounts of quaternized groups and quaternizable groups that are unquaternized) which is most suitable for the desired effect.

The polymer suitable for the use according to the invention comprises at most 99.99 and at least 10, preferably at least 30 and particularly preferably at least 60% by weight of the monomer(s) a).

Particularly for use of the polymers as thickeners in cosmetic formulations for the hair, the proportion of component a) incorporated during the polymerization is advantageously at least 60 and preferably at least 70% by weight.

Component b)

The polymer suitable for the use according to the invention comprises 0 to 90, preferably 10 to 70 and particularly preferably 30 to 60% by weight of at least one monoethylenically unsaturated compound b) containing amide groups which differs from a), incorporated during the polymerization.

This additional component b) is preferably selected from compounds of general formula IV:

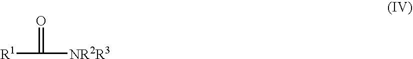

in which

R¹ is a group of the formula CH₂═CR⁴—, where R⁴═H or C₁-C₄-alkyl, and R² and R³ independently of one another are H, alkyl, cycloalkyl, heterocycloalkyl, aryl or heteroaryl, or R² and R³, together with the nitrogen atom to which they are bonded, are a five-membered to eight-membered nitrogen heterocycle, or

R² is a group of the formula CH₂═CR⁴— and R¹ and R³ independently of one another are H, alkyl, cycloalkyl, heterocycloalkyl, aryl or heteroaryl, or R¹ and R³, together with the amide group to which they are bonded, are a lactam having 5 to 8 ring atoms.

Preferably, the polymer used according to the invention additionally comprises, as monomer b), at least one N-vinyllactam incorporated during the polymerization. Suitable N-vinyllactams b) are unsubstituted N-vinyllactams and N-vinyllactam derivatives which can have e.g. one or more C₁-C₆-alkyl substituents such as methyl, ethyl, n-propyl, isopropyl, n-butyl, sec-butyl, tert-butyl, etc. These include e.g. N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam, etc., and mixtures thereof.

Preferably, the polymer suitable for the use according to the invention comprises incorporated monomers b) in which, in formula IV, R² is CH₂=CH— and R¹ and R³, together with the amide group to which they are bonded, are a lactam having 5 ring atoms.

It is particularly preferable to use N-vinylpyrrolidone, N-vinylcaprolactam, N-vinylformamide, (meth)acrylamide or mixtures thereof, N-vinylpyrrolidone and methacrylamide being very particularly preferred.

In one special embodiment the polymer suitable for the use according to the invention only comprises monomers a) and b) incorporated in it, a) preferably being N-vinyl-imidazole and b) preferably being N-vinylpyrrolidone.

The polymer suitable for the use according to the invention comprises at most 90, preferably at most 70 and particularly preferably at most 40% by weight of monomer(s) b) incorporated during the polymerization. In one embodiment of the invention the polymer suitable for the use according to the invention comprises at least 1, particularly preferably at least 10 and very particularly preferably at least 20% by weight of monomer(s) b) incorporated during the polymerization.

Crosslinking Agent c)

In another preferred embodiment of the invention the crosslinking agent c) used to prepare the polymers suitable for the use according to the invention is selected from compounds having at least 2 ethylenically unsaturated, non-conjugated double bonds per molecule.

Examples of suitable crosslinking agents c) are acrylic acid esters, methacrylic acid esters, allyl ethers or vinyl ethers of at least dihydric alcohols. The OH groups of the alcohols on which they are based can be completely or partially etherified or esterified, but the crosslinking agents comprise at least two ethylenically unsaturated groups.

Examples of the alcohols on which said crosslinking agents are based are dihydric alcohols such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexane-diol, 1,4-bis(hydroxymethyl)cyclohexane, hydroxypivalic acid neopentyl glycol monoester, 2,2-bis(4-hydroxyphenyl)propane, 2,2-bis[4-(2-hydroxypropyl)phenyl]-propane, diethylene glycol, triethylene glycol, tetraethylene glycol, dipropylene glycol, tripropylene glycol, tetrapropylene glycol and 3-thiopentane-1,5-diol, and polyethylene glycols, polypropylene glycols and polytetrahydrofurans having molecular weights of 200 to 10,000 in each case.

As well as ethylene oxide or propylene oxide homopolymers, it is also possible to use block copolymers of ethylene oxide or propylene oxide, or copolymers comprising incorporated ethylene oxide and propylene oxide groups.

Examples of alcohols on which said crosslinking agents are based and which have more than two OH groups are trimethylolpropane, glycerol, pentaerythritol, 1,2,5-pentanetriol, 1,2,6-hexanetriol, triethoxycyanuric acid, sorbitan, and sugars such as sucrose, glucose and mannose. Other preferred polyhydric alcohols in this context are disaccharides and trisaccharides.

Of course, the polyhydric alcohols can also be used after reaction with ethylene oxide or propylene oxide as the corresponding ethoxylates or propoxylates. The polyhydric alcohols can also be converted first to the corresponding glycidyl ethers by reaction with epichlorohydrin.

Other suitable crosslinking agents are the esters of vinyl alcohol or monohydric unsaturated alcohols with olefinically unsaturated C₃ to C₆ carboxylic acids, e.g. acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. It is also possible, however, to esterify the monohydric unsaturated alcohols with polybasic carboxylic acids, e.g. malonic acid, tartaric acid, trimellitic acid, phthalic acid, terephthalic acid, citric acid or succinic acid.

Other suitable crosslinking agents are esters of unsaturated carboxylic acids, e.g. oleic acid, crotonic acid, cinnamic acid or 10-undecenoic acid, with the polyhydric alcohols described above.

Other suitable crosslinking agents c) are linear, branched or cyclic aliphatic or aromatic hydrocarbons having at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane, or polybutadienes having molecular weights of 200 to 20,000.

Other suitable crosslinking agents are the amides of (meth)acrylic acid, itaconic acid and maleic acid with N-allylamines of at least dibasic amines. Examples of such amines are 1,2-diaminomethane, 1,2-diaminoethane, 1,3-diaminopropane, 1,4-diaminobutane, 1,6-diaminohexane, 1,12-dodecanediamine, piperazine, diethylene-triamine or isophoronediamine. Other suitable compounds are the amides of allylamine with unsaturated carboxylic acids such as acrylic acid, methacrylic acid, itaconic acid, maleic acid or at least dibasic carboxylic acids, as described above.

Triallylamine and triallylmonoalkylammonium salts, e.g. triallylmethylammonium chloride or methylsulfate, are also suitable as crosslinking agents.

Also suitable are N-vinyl compounds of urea derivatives, at least dibasic amides, cyanurates or urethanes, e.g. those of urea, ethyleneurea, propyleneurea or tartaric acid diamide, examples being N,N′-divinylethyleneurea or N,N′-divinylpropyleneurea.

Alkylenebisacrylamides such as methylenebisacrylamide, and N,N′-(2,2)butane and 1,1′-bis(3,3′-vinylbenzimidazolin-2-one)-1,4-butane are also suitable.

Examples of other suitable crosslinking agents are alkylene glycol di(meth)acrylates such as ethylene glycol diacrylate, ethylene glycol dimethacrylate, tetraethylene glycol acrylate, tetraethylene glycol dimethacrylate, diethylene glycol acrylate, diethylene glycol methacrylate, vinyl acrylate, allyl acrylate, allyl methacrylate, divinyldioxane, pentaerythrityl allyl ether and mixtures of these crosslinking agents.

Other suitable crosslinking agents are tetraallylsilane or tetravinylsilane.

Examples of crosslinking agents that are particularly preferably used are methylenebisacrylamide, triallylamine and triallylalkylammonium salts, divinylimidazole, pentaerythrityl triallyl ether, N,N′-divinylethyleneurea, reaction products of polyhydric alcohols with acrylic acid or methacrylic acid, and methacrylic acid esters and acrylic acid esters of polyalkylene oxides or of polyhydric alcohols which have been reacted with ethylene oxide and/or propylene oxide and/or epichlorohydrin.

Very particularly preferred crosslinking agents are pentaerythrityl triallyl ether, methylenebisacrylamide, N,N′-divinylethyleneurea, triallylamine and triallylmonoalkylammonium salts, and acrylic acid esters of ethylene glycol, butanediol, trimethylol-propane or glycerol, or acrylic acid esters of glycol, butanediol, trimethylolpropane or glycerol which has been reacted with ethylene oxide and/or epichlorohydrin. Pentaerythrityl triallyl ether is very particularly preferred.

Of course, it is also possible to use mixtures of the aforesaid compounds. The crosslinking agent is preferably soluble in the reaction medium. If the solubility of the crosslinking agent in the reaction medium is low, it can be dissolved in a monomer or a monomer mixture, or it can be metered in as a solution in a solvent that is miscible with the reaction medium. Particularly preferred crosslinking agents are those which are soluble in the monomer mixture.

The crosslinking agents c) are employed for the use according to the invention in amounts of at least 0.01, preferably of at least 0.05 and particularly preferably of at least 0.1, and of at most 5, preferably of at most 2 and particularly preferably of at most 1% by weight.

In one particularly preferred embodiment of the invention, pentaerythrityl triallyl ether is used in an amount of 0.1% by weight to 0.7% by weight and particularly preferably in an amount of 0.3% by weight to 0.6% by weight.

The amount of crosslinking agent c) in % by weight is based on the amount of the mixture of components a) to e) used to prepare the polymer.

Other monoethylenically Unsaturated Compound d)

According to the invention the mixture to be polymerized also comprises 0 to 15% by weight of at least one monoethylenically unsaturated compound d1) comprising at least one group selected from the group comprising optionally substituted C5-C30-alkyl, C5-C30-alkenyl, C5-C8-cycloalkyl, aryl, arylalkyl and heteroaryl, and/or a reactive precursor d2).

The component d) carries a hydrophobic group in the polymer suitable for the use according to the invention.

The compounds d1) can be intrinsically hydrophobic monomers such as esters or amides of (meth)acrylic acid with aliphatic C5 to C30 alcohols or amines, e.g. hexyl(meth)acrylate or hexyl(meth)acrylamide, n-heptyl(meth)acrylate or n-heptyl-(meth)acrylamide, n-octyl(meth)acrylate or n-octyl(meth)acrylamide, n-nonyl(meth)acrylate or n-nonyl(meth)acrylamide, n-decyl(meth)acrylate or n-decyl-(meth)acrylamide, n-undecyl(meth)acrylate or n-undecyl(meth)acrylamide, n-dodecyl(meth)acrylate or n-dodecyl(meth)acrylamide, n-tridecyl(meth)acrylate or n-tridecyl-(meth)acrylamide, n-tetradecyl(meth)acrylate or n-tetradecyl(meth)acrylamide, n-pentadecyl(meth)acrylate or n-pentadecyl(meth)acrylamide, n-hexadecyl(meth)acrylate or n-hexadecyl(meth)acrylamide, n-heptadecyl(meth)acrylate or n-heptadecyl(meth)acrylamide, n-octadecyl(meth)acrylate or n-octadecyl-(meth)acrylamide and n-nonadecyl(meth)acrylate or n-nonadecyl(meth)acrylamide.

Other possible examples of the compounds d1) are (meth)acrylic acid esters of polyalkylene glycols substituted by hydrophobic radicals, such as alkyl-substituted (meth)acrylic acid polyethylene glycol esters.

Also suitable as d1) are long-chain allyl or vinyl ethers such as C5-C30-alkyl vinyl ethers or C5-C30-alkenyl vinyl ethers.

Also suitable as d1) are polyisobutene derivatives containing olefinically unsaturated groups and polymerizable by free radical polymerization. Of these, preferred compounds d1) are e.g. reaction products of polyisobutenesuccinic anhydride (PIBSA) with hydroxyalkyl(meth)acrylates and of polyisobutenesuccinimide (PIBSA) with hydroxyalkyl(meth)acrylates.

WO 04/035635, p. 12, line 26 to p. 27, line 2, gives a detailed description of processes for the preparation of polyisobutene derivatives which can then be converted to appropriate compounds d) by means of conventional reactions with components comprising olefinically unsaturated groups. This description is incorporated here to the full extent by way of reference. Examples of polyisobutene derivatives which can be converted to appropriate compounds d) are the products commercially available under the trade name Glissopal® or Kerocom® (both from BASF).

In one particularly preferred embodiment of the invention, 2 to 10% by weight of octadecyl vinyl ether and/or behenyl acrylate and/or stearyl methacrylate are used as component d).

In another particularly preferred embodiment of the invention, esters of (meth)acrylic acid with polyethylene glycol mono-C₁₆-C₂₂-alkyl ethers are used as component d).

Preferred polyethylene glycol mono-C₁₆-C₂₂-alkyl ethers contain from 25 to 80 units of ethylene oxide per molecule.

Esters of (meth)acrylic acid with Lutensol® AT 25, Lutensol® AT 50 or Lutensol® AT 80, for example, can be used as such compounds d).

Methacrylic acid esters of ethoxylated C₁₆-C₁₈ fatty alcohol mixtures (containing e.g. 25 mol of ethylene oxide), such as those commercially available as PLEX®O-6877 or PLEX®O-6954 (Degussa), are also suitable.

Within the framework of the present invention, reactive precursors d2) are understood as meaning monomers polymerizable by free radical polymerization which, either before or after their incorporation during the polymerization, can be covalently linked by means of an optionally polymer-analogous reaction to at least one group selected from the group comprising optionally substituted C5-C30-alkyl, C5-C30-alkenyl, C5-C8-cycloalkyl, aryl, arylalkyl and heteroaryl. Monoethylenically unsaturated compounds carrying an epoxy group may be mentioned by way of example. These epoxy groups can be covalently linked by reaction with C5-C30 alcohols having a C5-C30-alkyl chain, e.g. after incorporation into a polymer during the polymerization.

Preferred compounds d) are selected from the group comprising C₁₈-C₃₀-alkyl(meth)acrylates and C₁₈-C₃₀-alkyl vinyl ethers.

The polymer suitable for the use according to the invention comprises at most 20, preferably at most 15 and particularly preferably at most 10, and preferably at least 1 and particularly preferably at least 2-10% by weight of the component(s) d) incorporated during the polymerization.

A proportion of at least 2 and preferably of at least 4% by weight of component d) incorporated during the polymerization is particularly advantageous for use of the polymers as thickeners in cosmetic formulations for the skin.

Polyether-Containing Compound f)

The polymer suitable for the use according to the invention is obtainable by polymerization in the presence of 0 to 70% by weight, based on the amount of components a) to e), of a polyether-containing compound f).

Suitable polyether-containing compounds f) are generally water-soluble or water-dispersible, non-ionic polymers containing polyalkylene glycol groups. The proportion of polyalkylene glycol groups is preferably at least 40% by weight, based on the total weight of the compound f). Polyalkylene glycols, polyesters based on polyalkylene glycols, and polyetherurethanes are examples of polyether-containing compounds f) which can be used.

The component f) is preferably a polyether from the group comprising polyalkylene oxides based on ethylene oxide, propylene oxide and butylene oxides, polytetrahydrofuran and polyglycerol. Depending on the type of monomeric structural units used for their preparation, the polyether-containing compounds f) contain the following structural units:

—(CH₂)₂—O—, —(CH₂)₃—O—, —(CH₂)₄—O—, —CH₂—CH(CH₃)—O—, —CH₂—CH(CH₂—CH₃)—O—, —CH₂—CHOR^{a}—CH₂—O—,

in which R^{a} is C₁-C₂₄-alkyl and preferably C₁-C₄-alkyl.

Both homopolymers and copolymers are suitable, it being possible for the copolymers to comprise the alkylene oxide units in a random distribution or as blocks.

The compounds f) can additionally contain bridging groups selected e.g. from:

—C(═O)—O—, —O—C(═O)—O—, —C(═O)—NR^{b}—, —O—C(═O)—NR^{b}—, —NR^{c}—(C═O)—NR^{b}—,

in which R^{b} and R^{c} independently of one another are hydrogen, C₁-C₃₀-alkyl, preferably C₁-C₄-alkyl, or cycloalkyl.

The polyethers f) preferably have a number-average molecular weight Mₙ of at least 300.

The polyethers f) preferably have general formula Va or Vb:

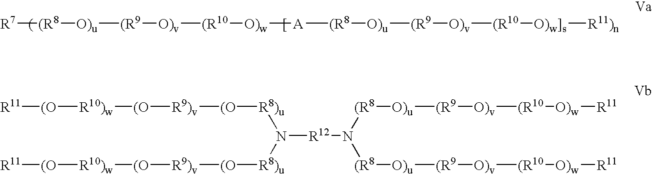

in which:
R⁷ is hydroxyl, amino, C₁-C₂₄-alkoxy, R¹³—COO—, R¹³—NH—COO— or a polyalcohol radical,
R⁸, R⁹ and R¹⁰ independently of one another are —(CH₂)₂—, —(CH₂)₃—, —(CH₂)₄—, —CH₂—CH(CH₃)—, —CH₂—CH(CH₂—CH₃)— or —CH₂—CHOR¹⁴—CH₂—,
R¹¹ is hydrogen, amino-C₁-C₆-alkyl, C₁-C₂₄-alkyl, R¹³—C(═O)— or R¹³—NH—C(═O)—,
R¹² is a C₁-C₂₀-alkylene group whose carbon chain can be interrupted by 1 to 10 non-adjacent oxygen atoms,
R¹³ is C₁-C₂₄-alkyl,
R¹⁴ is hydrogen, C₁-C₂₄-alkyl or R¹³—CO—,
A is —C(═O)—O—, —C(═O)—B—C(═O)—O— or —C(═O)—NH—B—NH—C(═O)—O—,
B is —(CH₂)ₜ—, optionally substituted cycloalkylene, optionally substituted heterocycloalkylene or optionally substituted arylene,
n is 1 or, if R⁷ is a polyalcohol radical, 1 to 8,
s is 0 to 500 and preferably 0 to 100,
t is 1 to 12 and preferably 2 to 6,
u independently of one another are each 1 to 5000 and preferably 1 to 1000,
v independently of one another are each 0 to 5000 and preferably 1 to 1000, and
w independently of one another are each 0 to 5000 and preferably 1 to 1000.

Preferred components f) are the polyethers of formula Va.

The terminal primary hydroxyl groups of the polyethers prepared on the basis of alkylene oxides, tetrahydrofuran or glycerol, and the secondary OH groups of polyglycerol can be either free or etherified with C₁-C₂₄ alcohols, esterified with C₁-C₂₄ carboxylic acids or converted to urethanes with isocyanates. Examples of alcohols suitable for this purpose are primary aliphatic alcohols such as methanol, ethanol, propanol and butanol, primary aromatic alcohols such as phenol, isopropylphenol, tert-butylphenol, octylphenol, nonylphenol and naphthol, secondary aliphatic alcohols such as isopropanol, tertiary aliphatic alcohols such as tert-butanol, and polyhydric alcohols, e.g. diols such as ethylene glycol, diethylene glycol, propylene glycol, 1,3-propanediol and butanediol, and triols such as glycerol and trimethylolpropane. However, the hydroxyl groups can also be replaced with primary amino groups by reductive amination, e.g. with hydrogen/ammonia mixtures under pressure, or converted to aminopropylene end groups by cyanoethylation with acrylonitrile and hydrogenation. Not only can the capping of the hydroxyl end groups take place later by reaction with the alcohols or with alkali metal hydroxide solutions, amines and hydroxy amines, but also these compounds can be used like Lewis acids, e.g. boron trifluoride, as starters at the beginning of the polymerization. Finally, the hydroxyl groups can also be capped by reaction with alkylating agents such as dimethyl sulfate.

The alkyl radicals in formulae Va and Vb can be branched or unbranched C₁-C₂₄-alkyl radicals, as initially defined, C₁-C₁₂-alkyl radicals being preferred and C₁-C₆-alkyl radicals being particularly preferred.

The average molecular weight Mₙ of the polyethers is at least 300 and at most 100,000. It is preferably 500 to 50,000, particularly preferably 2000 to 35,000 and very particularly preferably 2000 to 9000.

Advantageously, the base grafting material b) used consists of polytetrahydrofurans and homopolymers and copolymers of ethylene oxide, propylene oxide, butylene oxide and isobutylene oxide, which can be linear or branched. According to the invention the term homopolymers shall also embrace polymers which, apart from the polymerized alkylene oxide unit, still comprise the reactive molecules that were used for the initiation of the polymerization of the cyclic ethers or for the end group capping of the polymer.

Examples of preferred compounds f) are the polyether-containing compounds commercially available under the trade names Pluriol™, Pluronic™, Lutensol™, Pluracol™ and Plurafac™ (all from BASF), Lupranol™ (Elastogran) or PolyTHF® (BASF).

In general, polyol macromers can also be used as the component f). Such polyol macromers are known to those skilled in the art. The polyol macromers disclosed in U.S. Pat. No. 5,093,412 and WO 05/003200 may be cited in particular and they are incorporated here to the full extent by way of reference.

In one preferred embodiment of the invention the polymerization is carried out in the presence of silicones containing polyalkylene oxide as compounds f). Suitable silicones containing polyalkylene oxide are described e.g. in the following publications, whose disclosure is incorporated here to the full extent by way of reference: DE-PS 16 94 366: This relates to polysiloxane/polyoxyalkylene block copolymers whose polysiloxane block is synthesized in a manner known per se and whose polyoxyalkylene block consists of 25 to 70 percent by weight of a polyoxyalkylene having an average molecular weight of 1600 to 4000 and an ethylene oxide content of 20 to 100 percent by weight, the remainder being propylene oxide and optionally higher alkylene oxides, and 30 to 75 percent by weight of a polyoxyalkylene having an average molecular weight of 400 to 1200 and an ethylene oxide content of 65 to 100 percent by weight, the remainder being propylene oxide and optionally higher alkylene oxides.

DE-OS 25 41 865: The polysiloxane/polyoxyalkylene block copolymers are defined as follows in respect of their polyoxyalkylene blocks: one polyoxyalkylene block has an average molecular weight of 900 to 1300, and 30 to 55% by weight thereof consists of ethylene oxide, the remainder being propylene oxide, and the other polyoxyalkylene block has an average molecular weight of 3800 to 5000, and 30 to 50% by weight thereof consists of ethylene oxide, the remainder being propylene oxide.

EP-A 0 275 563: The block copolymer described comprises three different polyoxyalkylene blocks, namely one block comprising 20 to 60% by weight of oxyethylene units and having a molecular weight of 3000 to 5500, another block containing 20 to 60% by weight of oxyethylene units and having a molecular weight of 800 to 2900, and a third block consisting only of polyoxypropylene units and having a molecular weight of 130 to 1200.

Preferred silicones containing polyalkylene oxide are described in EP-A 0 670 342. On p. 3, line 22 to p. 4, line 56, EP-A 0 670 342 describes polysiloxanes comprising 1) at least two polyether radicals A and B, the polyoxyalkylene radical A having an average molecular weight of 600 to 5500 and consisting of 20 to 100% by weight of oxyethylene units and 80 to 0% by weight of oxypropylene units, and the polyoxyalkylene radical B having an average molecular weight of 700 to 5000 and consisting of 0 to <20% by weight of oxyethylene units and 100 to 80% by weight of oxypropylene units, and 2) Si-bonded hydrocarbon radicals having 6 to 30 carbon atoms.

Particularly suitable silicone derivatives are the compounds known by the INCl name Dimethicone Copolyols or silicone surfactants, e.g. those obtainable under the trade marks Abil® (Goldschmidt), Alkasil® (Rhône-Poulenc), Silicone Polyol Copolymer® (Genesee), Belsil® (Wacker), Silwet® (Witco) or Dow Corning® (Dow Corning). These comprise compounds with the CAS numbers 64365-23-7, 68937-54-2, 68938-54-5 and 68937-55-3.

Particularly suitable silicone derivatives are the compounds described on p. 10, line 24 to p. 12, line 8 and p. 13, line 3 to line 34 of WO 99/04750.

On p. 24, line 22 to p. 26, line 41, WO 01/013884 describes other particularly preferred silicones containing polyalkylene oxide.

The abovementioned publications and citations from the state of the art are incorporated to the full extent by way of reference.

Particularly preferably, the polymerization of the mixture of components a) to e) is carried out in the presence of 5 to 25% by weight of a polyethylene glycol having a molecular weight Mₙ of at least 2000 to at most 35,000, and preferably of at most 9000, and/or 5 to 25% by weight of esters of (meth)acrylic acid with polyethylene glycol mono-C₁₆-C₂₂-alkyl ethers, based in each case on the amount of components a) to e).

The polymerization of the mixture of components a) to e) is carried out in the presence of preferably at most 50 and particularly preferably at most 40% by weight of component f), based on the sum of the amounts of components a) to e).

In another embodiment of the invention the polymerization of the mixture of components a) to e) is carried out in the presence of 5 to 70, preferably 10 to 50 and particularly preferably 20 to 40% by weight of component f), based on the sum of the amounts of components a) to e).

Other Monomers e)

If desired, the polymers suitable for the use according to the invention can comprise 0 to 30% by weight of other monoethylenically unsaturated compounds differing from a) to d), incorporated during the polymerization.

These other monomers e) are preferably selected from the esters of α,β-ethylenically unsaturated monocarboxylic and dicarboxylic acids with C₁-C₃₀-alkanols, C₂-C₃₀-alkanediols and C₂-C₃₀-amino alcohols, amides of α,β-ethylenically unsaturated monocarboxylic and dicarboxylic acids with C₂-C₃₀-diamines and C₂-C₃₀-amino alcohols having a primary or secondary amino group, amides of α,β-ethylenically unsaturated monocarboxylic acids and N-alkyl and N,N-dialkyl derivatives thereof, N-vinylamides of saturated monocarboxylic acids, esters of vinyl alcohol and allyl alcohol with C₁-C₃₀-monocarboxylic acids, vinyl ethers, α,β-ethylenically unsaturated monocarboxylic and dicarboxylic acids, vinylaromatics, vinyl halides, vinylidene halides, C₁-C₈-monoolefins, non-aromatic hydrocarbons having at least two conjugated double bonds, and mixtures thereof.

Suitable additional monomers e) are methyl(meth)acrylate, methyl ethacrylate, ethyl(meth)acrylate, ethyl ethacrylate, tert-butyl(meth)acrylate, tert-butyl ethacrylate, n-octyl(meth)acrylate, 1,1,3,3-tetramethylbutyl(meth)acrylate, ethylhexyl(meth)acrylate, n-nonyl(meth)acrylate, n-decyl(meth)acrylate, n-undecyl(meth)acrylate, tridecyl(meth)acrylate, myristyl(meth)acrylate, pentadecyl(meth)acrylate, palmityl(meth)-acrylate, heptadecyl(meth)acrylate, nonadecyl(meth)acrylate, arachidyl(meth)-acrylate, behenyl(meth)acrylate, lignoceryl(meth)acrylate, cerotyl(meth)acrylate, melissyl(meth)acrylate, palmitoleyl(meth)acrylate, oleyl(meth)acrylate, linolyl(meth)acrylate, linolenyl(meth)acrylate, stearyl(meth)acrylate, lauryl(meth)acrylate and mixtures thereof.

Other suitable additional monomers e) are the esters of α,β-ethylenically unsaturated monocarboxylic and dicarboxylic acids with amino alcohols and preferably C₂-C₁₂-amino alcohols. These can preferably be C₁-C₈-monoalkylated or -dialkylated on the amine nitrogen. Examples of suitable acid components of these esters are acrylic acid, methacrylic acid, fumaric acid, maleic acid, itaconic acid, crotonic acid, maleic anhydride, monobutyl maleate and mixtures thereof. It is preferable to use acrylic acid, methacrylic acid and mixtures thereof. The following esters are preferred:

tert-butylaminoethyl(meth)acrylate, N,N-dimethylaminomethyl(meth)acrylate, N,N-di-methylaminoethyl(meth)acrylate, N,N-diethylaminoethyl(meth)acrylate, N,N-dimethyl-aminopropyl(meth)acrylate, N,N-diethylaminopropyl(meth)acrylate, N,N-dimethyl-aminocyclohexyl(meth)acrylate, etc.

Other suitable additional monomers e) are N-methyl(meth)acrylamide, N-ethyl-(meth)acrylamide, N-propyl(meth)acrylamide, N-(n-butyl)(meth)acrylamide, tert-butyl-(meth)acrylamide, n-octyl(meth)acrylamide, 1,1,3,3-tetramethylbutyl(meth)acrylamide, ethylhexyl(meth)acrylamide, n-nonyl(meth)acrylamide, n-decyl(meth)acrylamide, n-undecyl(meth)acrylamide, tridecyl(meth)acrylamide, myristyl(meth)acrylamide, pentadecyl(meth)acrylamide, palmityl(meth)acrylamide, heptadecyl(meth)acrylamide, nonadecyl(meth)acrylamide, arachidyl(meth)acrylamide, behenyl(meth)acrylamide, lignoceryl(meth)acrylamide, cerotyl(meth)acrylamide, melissyl(meth)acrylamide, palmitoleyl(meth)acrylamide, oleyl(meth)acrylamide, linolyl(meth)acrylamide, linolenyl(meth)acrylamide, stearyl(meth)acrylamide, N-lauryl(meth)acrylamide and mixtures thereof.

Other suitable additional monomers e) are 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate, 3-hydroxy-2-ethylhexyl methacrylate, etc.

Other suitable additional monomers e) are N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide and N-[4-(dimethylamino)cyclohexyl]-methacrylamide.

Other suitable additional monomers e) are acrylamide, methacrylamide, N-vinyl-formamide, N-vinylacetamide, N-vinylpropionamide and mixtures thereof.

Other suitable additional monomers e) are monoethylenically unsaturated monocarboxylic and dicarboxylic acids having 3 to 25 and preferably 3 to 6 C atoms, which can also be used in the form of their salts or anhydrides. Examples of these are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid, fumaric acid, 2-acrylamido-2-methylpropanesulfonic acid, vinylsulfonic acid, vinylsulfuric acid, vinylphosphoric acid, 10-undecenoic acid, 4-pentenoic acid, cinnamic acid, 3-butenoic acid, 5-hexenoic acid, 6-heptenoic acid, 7-octenoic acid, citraconic acid, mesaconic acid, styrenesulfonic acid, styrenesulfuric acid, 3-sulfopropyl acrylate, bis(3-sulfopropyl)itaconate, 3-sulfopropyl methacrylate, 3-allyloxy-2-hydroxy-propane-1-sulfonic acid, 2-acrylamido-2-methylethanesulfonic acid, 2-sulfoethyl acrylate, bis(2-sulfoethyl)itaconate, 2-sulfoethyl methacrylate, 3-sulfopropyl methacrylate, 3-allyloxy-2-hydroxypropane-1-sulfonic acid, 3-allyloxy-2-hydroxyethane-1-sulfonic acid and their alkali metal and ammonium salts, especially their sodium and potassium salts.

Other suitable additional monomers e) are ethylene, propylene, isobutylene, butadiene, styrene, α-methylstyrene, o-chlorostyrene, acrylonitrile, methacrylonitrile, vinyl chloride, vinylidene chloride, vinyl fluoride, vinylidene fluoride, ethers of vinyl alcohol and monoalcohols having 1 to 18 C atoms, e.g. methyl vinyl ether, and esters of vinyl alcohol and monocarboxylic acids having 1 to 18 C atoms, such as vinyl acetate, vinyl propionate, vinyl n-butyrate, vinyl laurate and vinyl stearate, and mixtures thereof.

Methyl(meth)acrylate is particularly preferred as the component e).

Polymerization

To prepare the polymers, the mixture of components a) to e) to be polymerized, in the presence of f), can be polymerized either with the aid of free radical-forming initiators or by the action of high-energy radiation, which shall also be understood as meaning the action of high-energy electrons.

The initiators used for the free radical polymerization can be the peroxo and/or azo compounds conventionally used for this purpose, e.g. alkali metal or ammonium peroxydisulfates, diacetyl peroxide, dibenzoyl peroxide, succinyl peroxide, ditert-butyl peroxide, tert-butyl perbenzoate, tert-butyl perpivalate, tert-butyl peroxy-2-ethyl-hexanoate, tert-butyl permaleate, cumene hydroperoxide, diisopropyl peroxydicarbamate, bis(o-toluoyl) peroxide, didecanoyl peroxide, dioctanoyl peroxide, dilauroyl peroxide, tert-butyl perisobutyrate, tert-butyl peracetate, ditert-amyl peroxide, tert-butyl hydroperoxide, azobisisobutyronitrile, azobis(2-amidinopropane)dihydro-chloride or 2,2′-azobis(2-methylbutyronitrile). Initiator mixtures or redox initiator systems are also suitable, examples being ascorbic acid/iron(II) sulfate/sodium peroxodisulfate, tert-butyl hydroperoxide/sodium disulfite and tert-butyl hydroperoxide/sodium hydroxymethanesulfinate.

It is preferable to use organic peroxides.

The polymerization can also be carried out by the action of ultraviolet radiation, optionally in the presence of UV initiators. Polymerization under the action of UV radiation is carried out with the photoinitiators or sensitizers conventionally used for this purpose. These are e.g. compounds such as benzoin and benzoin ether, α-methylbenzoin or α-phenylbenzoin. So-called triplet sensitizers, such as benzyl diketals, can also be used. As well as high-energy UV lamps such as carbon arc lamps, mercury vapor lamps or xenon lamps, examples of other UV radiation sources used are low-UV light sources such as fluorescent tubes with a high blue component.

The amounts of initiator or initiator mixtures used, based on the starting monomer, are between 0.01 and 10% by weight and preferably between 0.1 and 5% by weight.

The polymerization takes place at temperatures ranging from 40 to 200° C., preferably from 50 to 140° C. and particularly preferably from 60 to 110° C. It is conventionally carried out under atmospheric pressure, but it can also proceed under reduced or elevated pressure and preferably at between 1 and 5 bar.

The polymerization can be carried out e.g. as a solution polymerization, bulk polymerization, emulsion polymerization, reverse emulsion polymerization, suspension polymerization, reverse suspension polymerization or precipitation polymerization, but the methods which can be used are not restricted to this list.

Particularly preferably, the polymerization is carried out as a precipitation polymerization.

In the case of a bulk polymerization, a possible procedure is to dissolve the component f) in a mixture of the components a) to e) and, after the addition of a polymerization initiator, to polymerize the mixture to completion.

The polymerization can also be carried out semicontinuously by initially taking a fraction, e.g. 10%, of the mixture to be polymerized, consisting of component f), monomers a) to e) and initiator, heating the mixture to the polymerization temperature and, after the polymerization has started, adding the remainder of the mixture to be polymerized, as the polymerization advances. The polymers can also be obtained by placing the component f) in a reactor, heating it to the polymerization temperature and adding and polymerizing the mixture of monomers a) to e) and polymerization initiator, either all at once, in batches or, preferably, continuously.

If desired, the above-described polymerization can also be carried out in a solvent, examples of suitable solvents being alcohols such as methanol, ethanol, n-propanol, isopropanol, n-butanol, sec-butanol, tert-butanol, n-hexanol and cyclohexanol, glycols such as ethylene glycol, propylene glycol and butylene glycol, the methyl or ethyl ethers of dihydric alcohols, diethylene glycol, triethylene glycol, glycerol, dioxane, butyl acetate, ethyl acetate and toluene, particular preference being afforded to ethyl acetate, butyl acetate and mixtures thereof.

The polymerization can also be carried out with substances capable of controlling the molecular weight of the polymers, which generally are conventionally referred to as regulators.

Regulators

The free radical polymerization of the monomer mixture can take place in the presence of at least one regulator. Regulators are used in an amount preferably of 0.0005 to 5% by weight, particularly preferably of 0.001 to 2.5% by weight and very particularly preferably of 0.01 to 1.5% by weight, based on the total weight of components a) to e).

Regulators (polymerization regulators) are generally understood as meaning compounds with high transfer constants. Regulators accelerate chain transfer reactions and thereby reduce the degree of polymerization of the resulting polymers without influencing the overall reaction rate.

Regulators can be subdivided into mono-, bi- and polyfunctional regulators according to the number of functional groups in the molecule which are capable of leading to one or more chain transfer reactions. Suitable regulators are described in detail by e.g. K. C. Berger and G. Brandrup in J. Brandrup, E. H. Immergut, Polymer Handbook, 3rd edition, John Wiley & Sons, New York, 1989, pp II/81-II/141.

Examples of suitable regulators are aldehydes such as formaldehyde, acetaldehyde, propionaldehyde, n-butyraldehyde and isobutyraldehyde.

The following can also be used as regulators: formic acid, its salts or esters, such as ammonium formate, 2,5-diphenyl-1-hexene, hydroxyammonium sulfate and hydroxyammonium phosphate.

Other suitable regulators are halogen compounds, e.g. alkyl halides such as carbon tetrachloride, chloroform, bromotrichloromethane, bromoform and allyl bromide, and benzyl compounds such as benzyl chloride or benzyl bromide.

Other suitable regulators are allyl compounds, e.g. allyl alcohol, functionalized allyl ethers such as allyl ethoxylates, alkyl allyl ethers or glycerol monoallyl ether.

The regulators used are preferably compounds comprising sulfur in bonded form.

Examples of compounds of this type are inorganic hydrogen sulfites, disulfites and dithionites or organic sulfides, disulfides, polysulfides, sulfoxides and sulfones. These include di-n-butyl sulfide, di-n-octyl sulfide, diphenyl sulfide, thiodiglycol, ethylthio-ethanol, diisopropyl disulfide, di-n-butyl disulfide, di-n-hexyl disulfide, diacetyl disulfide, diethanol sulfide, di-t-butyl trisulfide, dimethyl sulfoxide, dialkyl sulfide, dialkyl disulfide and/or diary sulfide.

Organic compounds comprising sulfur in bonded form are particularly preferred.

Compounds which are preferably used as polymerization regulators are thiols (compounds containing sulfur in the form of SH groups, also called mercaptans). Preferred regulators are mono-, bi- and polyfunctional mercaptans, mercapto alcohols and/or mercapto carboxylic acids.

Examples of these compounds are allyl thioglycolates, ethyl thioglycolate, cysteine, 2-mercaptoethanol, 1,3-mercaptopropanol, 3-mercaptopropane-1,2-diol, 1,4-mercapto-butanol, mercaptoacetic acid, 3-mercaptopropionic acid, mercaptosuccinic acid, thioglycerol, thioacetic acid, thiourea, and alkylmercaptans such as n-butylmercaptan, n-hexylmercaptan or n-dodecylmercaptan.

Particularly preferred thiols are cysteine, 2-mercaptoethanol, 1,3-mercaptopropanol, 3-mercaptopropane-1,2-diol, thioglycerol and thiourea.

Examples of bifunctional regulators comprising two sulfurs in bonded form are bifunctional thiols such as dimercaptopropanesulfonic acid (sodium salt), dimercaptosuccinic acid, dimercapto-1-propanol, dimercaptoethane, dimercaptopropane, dimercaptobutane, dimercaptopentane, dimercaptohexane, ethylene glycol bisthioglycolates and butanediol bisthioglycolate.

Examples of polyfunctional regulators are compounds comprising more than two sulfurs in bonded form, e.g. trifunctional and/or tetrafunctional mercaptans.

Preferred trifunctional regulators are trifunctional mercaptans such as trimethylolpropane tris(2-mercaptoethanoate), trimethylolpropane tris(3-mercaptopropionate), trimethylolpropane tris(4-mercaptobutanoate), trimethylolpropane tris(5-mercaptopentanoate), trimethylolpropane tris(6-mercaptohexanoate), trimethylolpropane tris(2-mercaptoacetate), glyceryl thioglycolate, glyceryl thiopropionate, glyceryl thioethylate, glyceryl thiobutanoate, 1,1,1-propanetriyl tris(mercaptoacetate), 1,1,1-propanetriyl tris(mercaptoethanoate), 1,1,1-propanetriyl tris(mercaptopropionate), 1,1,1-propanetriyl tris(mercaptobutanoate), 2-hydroxmethyl-2-methyl-1,3-propanediol tris(mercaptoacetate), 2-hydroxmethyl-2-methyl-1,3-propanediol tris(mercaptoethanoate), 2-hydroxmethyl-2-methyl-1,3-propandiol tris(mercaptopropionate) and 2-hydroxmethyl-2-methyl-1,3-propanediol tris(mercaptobutanoate).

Particularly preferred trifunctional regulators are glyceryl thioglycolate, trimethylolpropane tris(2-mercaptoacetate) and 2-hydroxmethyl-2-methyl-1,3-propandiol tris(mercaptoacetate).

Preferred tetrafunctional mercaptans are pentaerythritol tetrakis(2-mercaptoacetate), pentaerythritol tetrakis(2-mercaptoethanoate), pentaerythritol tetrakis(3-mercaptopropionate), pentaerythritol tetrakis(4-mercaptobutanoate), pentaerythritol tetrakis-(5-mercaptopentanoate) and pentaerythritol tetrakis(6-mercaptohexanoate).

Other suitable polyfunctional regulators are Si compounds formed by reacting compounds of formula (IVa). Other suitable polyfunctional regulators are Si compounds of the formula

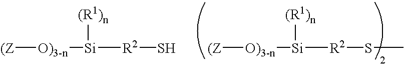

in which
n has a value of 0 to 2,
R¹ is a C₁-C₁₆-alkyl group or a phenyl group,
R² is a C₁-C₁₈-alkyl group or a cyclohexyl or phenyl group, and
Z is a C₁-C₁₈-alkyl group, C₂-C₁₈-alkylene group or C₂-C₁₈-alkynyl group whose carbon atoms can be replaced with non-adjacent oxygen or halogen atoms, or one of the groups

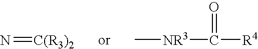

in which
R³ is a C₁-C₁₂-alkyl group and
R⁴ is a C₁-C₁₈-alkyl group.

All of said regulators can be used individually or in combination with one another.

In one embodiment of the invention no regulator is used.

Preferred polymers are those obtainable by the free radical polymerization of

99.99 to 10% by weight of component a), especially N-vinylimidazole,

0 to 90% by weight of component b), especially N-vinylpyrrolidone,

0.01 to 5% by weight of a crosslinking agent c), especially pentaerythrityl triallyl ether,

0 to 20% by weight of a component d), especially octadecyl vinyl ether and/or stearyl methacrylate, and

0 to 30% by weight of a component e), in the presence of

0 to 70% by weight, based on the sum of components a) to e), of a polyether-containing compound f), especially polyethylene glycol,

with the proviso that the amounts of components a) to e) add up to 100% by weight, the polymerization being carried out in the presence of less than 69% by weight of cyclohexane and less than 12% by weight of water, based on the total amount of all the components present during the polymerization, and in the absence of supercritical carbon dioxide.

Other preferred polymers are those obtainable by the free radical graft copolymerization of

97.95 to 40% by weight of component a), especially N-vinylimidazole,

1 to 60% by weight of component b), especially N-vinylpyrrolidone,

0.05 to 2% by weight of a crosslinking agent c), especially pentaerythrityl triallyl ether,

1 to 15% by weight of a component d), especially octadecyl vinyl ether and/or stearyl methacrylate, and

0 to 20% by weight of a component e), in the presence of

0 to 50% by weight, based on the sum of components a) to e), of a polyether-containing compound f), especially polyethylene glycol,

with the proviso that the amounts of components a) to e) add up to 100% by weight, the polymerization being carried out in the presence of less than 69% by weight of cyclohexane and less than 12% by weight of water, based on the total amount of all the components present during the polymerization, and in the absence of supercritical carbon dioxide.

Very particularly preferred polymers are those obtainable by the free radical graft copolymerization of

a) 96.9 to 60% by weight of component a), especially N-vinylimidazole,

b) 1 to 40% by weight of component b), especially N-vinylpyrrolidone and/or methacrylamide,

c) 0.1 to 1% by weight of a crosslinking agent c), especially pentaerythrityl triallyl ether,

d) 2 to 10% by weight of a component d), especially octadecyl vinyl ether and/or stearyl methacrylate and/or behenyl acrylate and/or esters of (meth)acrylic acid with polyethylene glycol mono-C₁₆-C₂₂-alkyl ethers, and

e) 0 to 10% by weight of a component e), in the presence of

f) 0 to 35% by weight, based on the sum of components a) to e), of a polyether-containing compound f), especially polyethylene glycol and/or polyethylene glycol mono-C₁₆-C₂₂-alkyl ether and/or polytetrahydrofuran,

with the proviso that the amounts of components a) to e) add up to 100% by weight, the polymerization being carried out in the presence of less than 69% by weight of cyclohexane and less than 12% by weight of water, based on the total amount of all the components present during the polymerization, and in the absence of supercritical carbon dioxide.

The preferred method of preparing the polymers suitable for the use according to the invention is precipitation polymerization. This polymerization method uses solvents in which the starting materials for the polymerization are soluble and the polymer formed is insoluble. Examples of suitable solvents are aromatic hydrocarbons such as toluene, xylenes and benzene, or aliphatic hydrocarbons such as n-alkanes and cyclohexane, acetic acid esters such as ethyl acetate and butyl acetate, ethers such as diethyl ether, dipropyl ether, dibutyl ether, methyl tert-butyl ether and diethylene glycol dimethyl ether, ketones such as acetone and methyl ethyl ketone, and mixtures of these solvents. Mixtures of e.g. ethyl acetate and butyl acetate are particularly suitable because in this solvent mixture the polymers are obtained in a form that can readily be separated off (sedimentation is accelerated), and moreover because the reaction temperature in mixtures of butyl acetate and ethyl acetate can be chosen above the boiling point of ethyl acetate, with simultaneous cooling due to the boiling ethyl acetate.

The polymerization is carried out in the presence of less than 69% by weight of cyclohexane and less than 12% by weight of water, based on the total amount of all the components present during the polymerization.

The polymerization is carried out in the presence of preferably less than 50, particularly preferably less than 40 and very particularly preferably less than 30% by weight of cyclohexane.

The polymerization is carried out in the presence of preferably less than 10, particularly preferably less than 8 and very particularly preferably less than 5% by weight of water.

In one preferred embodiment, at least 30, preferably 50 and particularly preferably 70% by weight of the solvent used for the polymerization consists of ethyl acetate or n-butyl acetate or mixtures thereof.

The initiators used can be any of those also used in solution polymerization. It is preferable to use 0.01 to 1.5% by weight of initiator, based on the starting monomers.

The precipitation polymerization is conventionally carried out at temperatures of 20 to 150° C., preferably of 40 to 120° C. and particularly preferably of 60 to 100° C.

The precipitation polymerization is conventionally carried out at pressures of 1 to 15 bar and especially of 1 to 6 bar.

The solvent or solvent mixture determines the maximum reaction temperature according to the corresponding boiling points, provided the polymerization is carried out under atmospheric pressure. However, polymerization under pressure is also possible.

In general the precipitation polymerization can be carried out with solids contents of up to approx. 40% by weight, the preferred range being between 25 and 40% by weight. Particularly in the case of high solids contents, it is advisable to carry out the polymerization in the presence of a protective colloid polymer. Suitable protective colloid polymers are those which dissolve readily in the solvents used and do not react with the monomers. Examples of polymers suitable as protective colloids are copolymers of maleic acid with vinyl alkyl ethers and/or olefins having 8 to 20 C atoms, or corresponding copolymers of maleic acid half-esters with C10-C20 alcohols, or monoamides and diamides of maleic acid with C10-C20-alkylamines, and polyvinyl alcohol ethers containing alkyl groups having 1 to 20 C atoms, or polyvinyl methyl, ethyl, isobutyl or octadecyl ether. The amount of protective colloid polymer used normally ranges from 0.05 to 4% by weight (based on monomers) and preferably from 0.1 to 2% by weight. It is often advantageous to use mixtures of several protective colloid polymers.

The polymerization is carried out by taking the solvent, component f), protective colloid polymer and optionally crosslinking agent c), heating them and effecting the polymerization by adding initiator and monomers a), b), d) and e) (optionally dissolved in the same solvent or solvent mixture). Another possibility, however, is to take fractions of the monomers and initiator (e.g. 10%), heat this mixture to the polymerization temperature and, after the reaction has started, add the remainder of the mixture to be polymerized, as the polymerization advances. It is also possible initially to introduce a fraction of the crosslinking agent used and add the remainder together with the rest of the components. In the case of lower solids contents, it is also conceivable to introduce all the starting materials into a batch reaction.

In one preferred embodiment the polymerization for the preparation of the polymers suitable for the use according to the invention is carried out by a fed batch method. Here, all or part of the individual reactants or of all the reactants is added to a reaction mixture batchwise or continuously and together or in separate feed streams. Separate feed streams are advantageous e.g. when the solubilities of components a) to e) in specific solvents are markedly different. For example, in the copolymerization of vinylimidazole as a) and methacrylamide as b) with the other components c) to e), a method with separate feed streams is advantageous because vinylimidazole and methacrylamide have very different solubilities.

The monomers and initiator are generally metered in over a period of 1 to 10 hours and preferably of 2 to 5 hours.

Other polymers can optionally also be present in the polymerization for the preparation of the polymers suitable for the use according to the invention, examples being polyamides, polyurethanes, polyesters, and homopolymers and copolymers of ethylenically unsaturated monomers. Examples of such polymers, some of which are also used in cosmetics, are the polymers known by the trade names Amerhold™, Ultrahold™, Ultrahold Strong™, Luviflex™ VBM, Luvimer™, Acronal™, Acudyne™, Stepanhold™, Lovocryl™, Versatyl™, Amphomer™ or Eastman AQ™, Luviset™ marks, Sokalan™ marks and Luviquat™ marks.

The precipitated polymer is then isolated from the reaction mixture, which can be done using any general method of isolating polymers in conventional precipitation polymerization. Such methods are filtration, centrifugation, solvent evaporation or combinations of these methods. For further purification, the polymer is washed to remove unpolymerized constituents. In principle, the same solvents as those suitable for the polymerization can be used for this purpose. If the polymers are to be alkylated after the polymerization, it is advantageous to use the same solvent for polymerization and alkylation.

If the polymer is to be dried, it is recommended to change the solvent after polymerization or alkylation and to use low-boiling solvents, e.g. acetone, for the drying.

Neutralization

In one preferred embodiment of the invention, before being used according to the invention in aqueous compositions, the polymer is neutralized after the polymerization and before or after the filtration.

Acids or bases may be required for the neutralization, depending on the choice of monomers a) to e).

Organic or inorganic acids are used as neutralizing agents for monomers carrying basic groups.

Possible organic acids which may be mentioned are monobasic and polybasic, optionally substituted aliphatic and aromatic carboxylic acids, monobasic and polybasic, optionally substituted aliphatic and aromatic sulfonic acids, monobasic and polybasic, optionally substituted aliphatic and aromatic phosphonic acids, polymers carrying acid groups, or ascorbic acid.

Preferred organic acids are hydroxycarboxylic acids, i.e. carboxylic acid derivatives in which one or more H atoms have been replaced by hydroxyl groups.

Glycolic acid, lactic acid, tartaric acid and citric acid may be mentioned as examples of hydroxycarboxylic acids.

Preferred inorganic acids which may be mentioned are phosphoric acid, phosphorous acid, sulfuric acid, sulfurous acid and hydrochloric acid.

Examples of neutralizing agents which can be used for monomers carrying acid groups are mineral bases such as sodium carbonate, alkali metal hydroxides and ammonia, and organic bases such as amino alcohols, especially 2-amino-2-methyl-1-propanol, monoethanolamine, diethanolamine, triethanolamine, triisopropanolamine, tri[(2-hydroxy)-1-propyl]amine, 2-amino-2-methyl-1,3-propanediol and 2-amino-2-hydroxymethyl-1,3-propanediol, and diamines, e.g. lysine.

Modification of the Rheological Properties

Modification of the rheological properties is understood quite generally as meaning the change in the deformation behavior and flow behavior of matter. The most important rheological properties are viscosity, thixotropy, structural viscosity, rheopexy and dilatancy. These terms are known to the person skilled in the art.

Viscosity is usually understood as meaning the “ropiness” of a liquid. It results from the intermolecular forces in a liquid, and is thus dependent on cohesion (intramolecular) and adhesion (intermolecular). The viscosity characterizes the flow behavior of a liquid. High viscosity means thick-liquid, whereas low viscosity means thin-liquid.

Thixotropy is usually understood as meaning the property of a fluid to exhibit a lower viscosity after shearing and to build up the original viscosity when motionless.

Rheopexy is usually understood as meaning the property of a fluid to exhibit a higher viscosity after shearing. This behavior is closely related to the dilatancy, in the case of which the viscosity is higher only during shearing.

Modification of the rheology is, in the context of this invention, in particular understood as meaning, the increase in the viscosity of liquids, usually also referred to as “thickening”. This viscosity increase can extend to the formation of gels or solids.

Aqueous Compositions

The invention also provides cosmetic or pharmaceutical products comprising at least one polymer defined as above, suitable for the use according to the invention.

Preferred products are aqueous compositions comprising the at least one polymer in an amount ranging from 0.01 to 5% by weight.

Particularly preferred products are aqueous cosmetic compositions (for the hair).

The polymers described above are outstandingly suitable for the preparation of cosmetic and pharmaceutical products, where they serve e.g. as polymeric film-forming agents in body care formulations, comprising the application of cosmetic formulations to keratinous surfaces such as the skin, hair and nails, as well as oral care preparations. They can be universally used and formulated in a wide variety of cosmetic compositions and are compatible with the conventional components. The polymers suitable for the use according to the invention can develop special effects in the cosmetic formulations. The polymers can contribute inter alia to retaining moisture in the skin, conditioning the skin and improving the feel of the skin.

The polymers act especially as thickeners and conditioners in the formulations.

In certain formulations the addition of the polymers suitable for the use according to the invention can substantially improve the skin tolerance.

In addition to the polymers suitable for the use according to the invention, the products according to the invention contain at least one cosmetically or pharmaceutically acceptable carrier B) selected from
i) water,
ii) water-miscible organic solvents, preferably C₁-C₄-alkanols,
iii) oils, fats and waxes,
iv) esters of C₆-C₃₀-monocarboxylic acids with mono-, di- or trihydric alcohols, which differ from iii),
v) saturated acyclic and cyclic hydrocarbons,
vi) fatty acids,
vii) fatty alcohols and
viii) mixtures thereof.

The compositions have, for example, an oil or fat component B) which is chosen from: hydrocarbons of low polarity, such as mineral oils; linear saturated hydrocarbons, preferably having more than 8 carbon atoms, such as tetradecane, hexadecane, octadecane etc.; cyclic hydrocarbons, such as decahydronaphthalene; branched hydrocarbons; animal and vegetable oils; waxes; wax esters; Vaseline; esters, preferably esters of fatty acids, such as, for example, the esters of C₁-C₂₄-mono-alcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexancosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkyl benzoates, benzyl benzoate; other cosmetic esters, such as fatty acid triglycerides, propylene glycol monolaurate, polyethylene glycol monolaurate, C₁₀-C₁₅-alkyl lactates, etc. and mixtures thereof.

Suitable silicone oils B) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are commercially available, for example, under the name cyclomethicone.

Preferred oil and fat components B) are chosen from paraffin and paraffin oils; Vaseline; natural fats and oils, such as castor oil, soya oil, peanut oil, olive oil, sunflower oil, sesame oil, avocado oil, coco butter, almond oil, peach kernel oil, resinous oil, cod-liver oil, lard, spermaceti, spermaceti oil, sperm oil, wheat germ oil, macadamia nut oil, evening primrose oil, jojoba oil; fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic acid and saturated, unsaturated, and substituted fatty acids different therefrom; waxes, such as beeswax, carnauba wax, candililla wax, spermaceti and mixtures of the abovementioned oil and fat components.

Suitable cosmetically and pharmaceutically compatible oil and fat components B) are described in Karl-Heinz Schrader, Grundlagen und Rezepturen der Kosmetika [Fundamentals and formulations of cosmetics], 2nd Edition, Verlag Hüthig, Heidelberg, pp. 319-355, which is hereby incorporated by reference.

Suitable hydrophilic carriers B) are chosen from water, 1-, 2- or polyhydric alcohols having preferably 1 to 8 carbon atoms, such as ethanol, n-propanol, isopropanol, propylene glycol, gycerol, sorbitol, etc.

The cosmetic compositions may be skin cosmetic or hair cosmetic compositions.

Preferably, the compositions are in the form of a spray, gel, foam, ointment, cream, emulsion, suspension, lotion, milk or paste. If desired, liposomes or microspheres can also be used.

The cosmetic, dermatological or pharmaceutical compositions can additionally comprise cosmetically, dermatologically or pharmaceutically active ingredients, and auxiliaries.

Preferably, the compositions comprise at least one polymer, as defined above, which is suitable for the use according to the invention, at least one carrier B) as defined above and at least one constituent different therefrom which is chosen from cosmetically active ingredients, emulsifiers, surfactants, preservatives, perfume oils, further thickeners, hair polymers, hair and skin conditioners, graft polymers, water-soluble or dispersible silicone-containing polymers, photoprotective agents, bleaches, gel formers, care agents, colorants, tinting agents, tanning agents, dyes, pigments, consistency regulators, humectants, refatting agents, collagen, protein hydrolysates, lipids, antioxidants, antifoams, antistats, emollients and softeners.

Thickeners

The cosmetic, dermatological or pharmaceutical compositions can, in addition to the polymer which is suitable for the use according to the invention, also comprise further thickeners. However, it is preferred to use no further thickeners.

Typical thickeners in such formulations are crosslinked polyacrylic acids and derivatives thereof, polysaccharides and derivatives thereof, such as xanthan gum, agar agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidones. Preference is given to using nonionic thickeners.

Cosmetically and/or Dermatologically Active Ingredients

Suitable cosmetically and/or dermatologically active ingredients are, for example, coloring active ingredients, skin and hair pigmentation agents, tinting agents, tanning agents, bleaches, keratin-hardening substances, antimicrobial active ingredients, photofilter active ingredients, repellent active ingredients, hyperemic substances, keratolytic and keratoplastic substances, antidandruff active ingredients, antiphlogistics, keratinizing substances, active ingredients which are antioxidative or act as free-radical scavengers, substances which moisturize the skin or retain moisture in the skin, refatting active ingredients, antierythimatos or antiallergic active ingredients and mixtures thereof.

Active ingredients which tan the skin artificially which are suitable for tanning the skin without natural or artificial irradiation with UV rays are, for example, dihydroxyacetone, alloxan and walnut shell extract. Suitable keratin-hardening substances are generally active ingredients as are also used in antiperspirants, such as, for example, potassium aluminum sulfate, aluminum hydroxychloride, aluminum lactate, etc. Antimicrobial active ingredients are used in order to destroy microorganisms and/or to inhibit their growth and thus serve both as preservatives and also as a deodorizing substance which reduces the formation or the intensity of body odor. These include, for example, customary preservatives known to the person skilled in the art, such as p-hydroxybenzoic esters, imidazolidinylurea, formaldehyde, sorbic acid, benzoic acid, salicylic acid etc. Such deodorizing substances are, for example, zinc ricinoleate, triclosan, undecylenic acid alkylolamides, triethyl citrate, chlorhexidine etc.

Suitable photofilter active ingredients are substances which absorb UV rays in the UV-B and/or UV-A region. Suitable UV filters are, for example, 2,4,6-triaryl-1,3,5-triazines in which the aryl groups may in each case carry at least one substituent which is preferably chosen from hydroxy, alkoxy, specifically methoxy, alkoxycarbonyl, specifically methoxycarbonyl and ethoxycarbonyl and mixtures thereof. Also suitable are p-aminobenzoic esters, cimminic esters, benzophenones, camphor derivatives, and pigments which deflect UV rays, such as titanium dioxide, talc and zinc oxide.

Photoprotective agents suitable for use in the compositions comprising water are all of the compounds specified in EP-A 1 084 696 in the paragraphs [0036] to [0053], which is hereby incorporated in its entirety by reference.

The list of specified UV photoprotective filters which can be used in the preparations according to the invention is not of course intended to be limiting.

Antimicrobial Agents

In addition, antimicrobial agents can also be used in the compositions comprising water. These generally include all suitable preservatives with a specific effect against gram-positive bacteria, e.g. triclosan (2,4,4′-trichlor-2′-hydroxydiphenyl ether), chlorhexidine (1,1′-hexamethylenebis[5-(4-chlorophenyl)biguanide) and TTC (3,4,4′-trichlorocarbanilide). Commercially available products are Phenonip®, Euxyl®400, Euxyl®100 or Euxyl®500.

Quaternary ammonium compounds are in principle likewise suitable, but are preferably used for disinfecting soaps and washing lotions.

Numerous fragrances also have antimicrobial properties. Special combinations with particular effectiveness against gram-positive bacteria are used for the composition of so-called deodorant perfumes.

A large number of essential oils or their characteristic ingredients, such as, for example, oil of cloves (eugenol), mint oil (menthol) or thyme oil (thymol), also exhibit marked antimicrobial effectiveness.

The antibacterially effective substances are generally used in concentrations of from about 0.1 to 0.3% by weight.

Suitable repellant active ingredients are compounds which are able to repel or drive away animals, in particular insects, from people. These include, for example, 2-ethyl-1,3-hexanediol, N,N-diethyl-m-toluamide etc.

Suitable hyperemic substances, which promote circulation in the skin, are, for example, essential oils, such as dwarf pine needle, lavender, rosemary, juniper berry, horse chestnut extract, birch leaf extract, hayflower extract, ethyl acetate, camphor, menthol, peppermint oil, rosemary extract, eucalyptus oil, etc.

Suitable keratolytic and keratoplastic substances are, for example, salicylic acid, calcium thioglycolate, thioglycolic acid and its salts, sulfur, etc.

Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics which counteract skin irritations are, for example, allantoin, bisabolol, dragosantol, Camille extract, panthenol, etc.

The cosmetic, dermatological or pharmaceutical compositions can comprise, as cosmetic and/or pharmaceutical active ingredient (and also if appropriate as auxiliary), at least one cosmetically or pharmaceutically acceptable polymer.

Preference is given to compositions which additionally comprise at least one nonionic, one anionic, one cationic or one ampholytic polymer.

Anionic polymers preferred as additional polymers are, for example, homopolymers and copolymers of acrylic acid and methacrylic acid and salts thereof. These also include crosslinked polymers of acrylic acid, as are available under the INCl name Carbomer. Such crosslinked homopolymers of acrylic acid are, for example, commercially available under the name Carbopol® (Noveon). Preference is also given to hydrophobically modified crosslinked polyacrylate polymers, such as Carbopol®Ultrez 21 (Noveon).

Further examples of suitable additional anionic polymers are copolymers of acrylic acid and acrylamide and salts thereof, sodium salts of polyhydroxycarboxylic acids, water-soluble or water-dispersible polyesters, polyurethanes and polyureas.

Preference is also given to compositions which additionally comprise a polyurethane as anionic polymer.

Particularly suitable additional polymers are the water-soluble or water-dispersible polyurethanes described in DE 4225045 A1, which is hereby incorporated in its entirety by reference. Of particular suitability is Luviset®P.U.R. (BASF).

In addition, particular preference is given to silicone-containing polyurethanes as are described in DE 19807908 A1, which is hereby incorporated in its entirety by reference. Of particular suitability is Luviset®Si-P.U.R. (BASF).

Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers which are obtainable from Rohm and Haas under the name Aculyn®. Particularly suitable polymers are also copolymers of t-butyl acrylate, ethyl acrylate, methacrylic acid (e.g. Luvimer®100P), copolymers of ethyl acrylate and methacrylic acid (e.g. Luviumer® MAE), copolymers of N-tert-butyl acrylamide, ethyl acrylate, acrylic acid (Ultrahold®8, strong), copolymers of vinyl acetate, crotonic acid and, if appropriate, further vinyl esters (e.g. Luviset® grades), maleic anhydride copolymers, if appropriate reacted with alcohol, anionic polysiloxanes, e.g. carboxyfunctional ones, t-butyl acrylate, methacrylic acid (e.g. Luviskol®VBM), copolymers of acrylic acid and methacrylic acid with hydrophobic monomers, such as, for example, C₄-C₃₀-alkyl esters of meth(acrylic acid), C₄-C₃₀-alkyl vinyl esters, C₄-C₃₀-alkyl vinyl ethers and hyaluronic acid. Examples of anionic polymers are also vinyl acetate/crotonic acid copolymers, as are sold, for example, under the names Resyn® (National Starch) and Gafset® (GAF) and vinylpyrrolidone/vinyl acrylate copolymers obtainable, for example, under the trade name Luviflex® (BASF). Further suitable polymers are the vinylpyrrolidone/acrylate terpolymer available under the name Luviflex®VBM-35 (BASF) and sodium sulfonate-containing polyamides or sodium sulfonate-containing polyesters.

The group of suitable anionic polymers also comprises, by way of example, Balance® CR (National Starch; Acrylate Copolymer), Balance® 0/55 (National Starch; Acrylate Copolymer), Balance® 47 (National Starch; Octylacrylamide/Acrylate/Butylaminoethyl Methacrylate Copolymer), Aquaflex® FX 64 (ISP; Isobutylene/Ethylmaleimide/Hydroxyethylmaleimide Copolymer), Aquaflex® SF-40 (ISP/National Starch; VP/Vinylcaprolactam/DMAPA Acrylate Copolymer), Allianz® LT-120 (ISP; Rohm & Haas; Acrylate/C1-2 Succinate/Hydroxyacrylate Copolymer), Aquarez® HS (Eastman; Polyester-1), Diaformer® Z-400 (Clariant; Methacryloylethylbetaine/Methacrylate Copolymer), Diaformer® Z-711 (Clariant; Methacryloylethyl N-Oxide/Methacrylate Copolymer), Diaformer® Z-712 (Clariant; Methacryloylethyl N-Oxide/Methacrylate Copolymer), Omnirez® 2000 (ISP; Monoethyl Ester of Poly(Methyl Vinyl Ether/Maleic Acid in Ethanol), Amphomer® HC (National Starch; Acrylate/Octylacrylamide Copolymer), Amphomer® 28-4910 (National Starch; Octylacrylamide/Acrylate/Butyl-aminoethyl Methacrylate Copolymer), Advantage® HC 37 (ISP; Terpolymer of Vinylcaprolactam/Vinylpyrrolidone/Dimethylaminoethyl Methacrylate), Advantage® LC55 and LC80 or LC A and LC E, Advantage® Plus (ISP; VA/Butyl Maleate/Isobornyl Acrylate Copolymer), Aculyne® 258 (Rohm & Haas; Acrylate/Hydroxy Ester Acrylate Copolymer), Luviset® P.U.R. (BASF, Polyurethane-1), Luviflex® Silk (BASF), Eastman® AQ 48 (Eastman), Styleze® CC-10 (ISP; VP/DMAPA Acrylates Copolymer), Styleze® 2000 (ISP; VP/Acrylates/Lauryl Methacrylate Copolymer), DynamX® (National Starch; Polyurethane-14 AMP-Acrylates Copolymer), Resyn® XP (National Starch; Acrylates/Octylacrylamide Copolymer), Fixomer® A-30 (Ondeo Nalco; Polymethacrylic Acid (and) Acrylamidomethylpropanesulfonic Acid), Fixate® G-100 (Noveon; AMP-Acrylates/Allyl Methacrylate Copolymer).

Suitable additional polymers are also the terpolymers of vinylpyrrolidone, C₁-C₁₀-alkyl, cycloalkyl and aryl(meth)acrylates and acrylic acid described in U.S. Pat. No. 3,405,084. Suitable additional polymers are also the terpolymers of vinylpyrrolidone tert-butyl meth)acrylate and (meth)acrylic acid described in EP-A-0 257 444 and EP-A-0 480 280. Suitable additional polymers are also the copolymers described in DE-A-42 23 066 which comprise at least one (meth)acrylic ester, (meth)acrylic acid, and N-vinylpyrrolidone and/or N-vinylcaprolactam in copolymerized form. The disclosure of these documents is hereby incorporated by reference.

Suitable polymers comprising carboxylic acid groups are also polyurethanes comprising carboxylic acid groups.

EP-A-636361 discloses suitable block copolymers with polysiloxane blocks and polyurethane/polyurea blocks which have carboxylic acid and/or sulfonic acid groups. Suitable silicone-containing polyurethanes are also described in WO 97/25021 and EP-A-751 162. Suitable polyurethanes are also described in DE-A-42 25 045, which is hereby incorporated in its entirety by reference.

These polyurethanes are in principle constructed from
i) at least one compound which comprises two or more active hydrogen atoms per molecule,
ii) at least one diol comprising carboxylic acid groups, or a salt thereof and
iii) at least one polyisocyanate.

Component i) is, for example, a diol, diamine, amino alcohol, or mixture thereof. The molecular weight of these compounds is preferably in a range from about 56 to 280. If desired, up to 3 mol % of said compounds can be replaced by triols or triamines.

Diols i) which can be used on, for example, ethylene glycol, propylene glycol, butylene glycol, neopentyl glycol, cyclohexanedimethylol, di-, tri-, tetra-, penta- or hexaethylene glycol and mixtures thereof. Preference is given to using neopentyl glycol and/or cyclohexanedimethylol. Suitable amino alcohols i) are, for example, 2-aminoethanol, 2-(N-methylamino)ethanol, 3-aminopropanol, 4-aminobutanol, 1-ethylaminobutan-2-ol, 2-amino-2-methyl-1-propanol, 4-methyl-4-aminopentan-2-ol etc. Suitable diamines i) are, for example, ethylenediamine, propylenediamine, 1,4-diaminobutane, 1,5-diamino-pentane and 1,6-diaminohexane, and α,ω-diaminopolyethers which can be prepared by amination of polyalkylene oxides with ammonia.

Component i) may also be a polymer with a number-average molecular weight in the range from about 300 to 5000, preferably about 400 to 4000, in particular 500 to 3000. Polymers i) which can be used are, for example, polyesterdiols, polyetherols and mixtures thereof. Polyetherols are preferably polyalkylene glycols, e.g. polyethylene glycols, polypropylene glycols, polytetrahydrofurans etc., block copolymers of ethylene oxide and propylene oxide or block copolymers of ethylene oxide, propylene oxide and butylene oxide, which comprise the copolymerized alkylene oxide units in random distribution or in the form of blocks. Suitable polytetrahydrofurans i) can also be prepared by cationic polymerization of tetrahydrofuran in the presence of acidic catalysts, such as, for example, sulfuric acid or fluorosulfuric acid. Such preparation methods are known to the person skilled in the art. Polyesterdiols i) which can be used preferably have a number-average molecular weight in the range from about 400 to 5000, preferably 500 to 3000, in particular 600 to 2000. Suitable polyesterdiols i) which can be used are all those which are usually used for producing polyurethanes, in particular those based on aromatic dicarboxylic acids, such as terephthalic acid, isophthalic acid, phthalic acid, Na or K sulfoisophthalic acid etc., aliphatic dicarboxylic acids, such as adipic acid or succinic acid etc., and cycloaliphatic dicarboxylic acids, such as 1,2-, 1,3- or 1,4-cyclohexanedicarboxylic acid. Suitable diols are, in particular, aliphatic diols, such as ethylene glycol, propylene glycol, 1,6-hexanediol, neopentyl glycol, diethylene glycol, polyethylene glycols, polypropylene glycols, 1,4-dimethylol-cyclohexane, etc.

Suitable compounds ii) which have two active hydrogen atoms and at least one carboxylic group per molecule are, for example, dimethylolpropanoic acid and mixtures which comprise dimethylolpropanoic acid.

Component iii) is a customary aliphatic, cycloaliphatic and/or aromatic polyisocyanate, such as tetramethylene diisocyanate, hexamethylene diisocyanate, methylenediphenyl diisocyanate, 2,4- and 2,6-tolylene diisocyanate, and isomer mixtures thereof, o- and m-xylylene diisocyanate, 1,5-naphthylene diisocyanate, 1,4-cyclohexylene diisocyanate, dicyclohexylmethane diisocyanate and mixtures thereof, in particular isophorone diisocyanate and/or dicyclohexylmethane diisocyanate. If desired, up to 3 mol % of said compounds can be replaced by triisocyanates.

Suitable additional polymers are also cationic polymers. These include, for example, polymers with the INCl name Polyquaternium, e.g. copolymers of vinylpyrrolidone/N vinylimidazolium salts (Luviquat™ FC, Luviquat™ HM, Luviquat™ MS, Luviquat™ Care), copolymers of N-vinylpyrrolidone/dimethylaminoethyl methacrylate, quaternized with diethyl sulfate (Luviquat™ PQ 11), copolymers of N-vinylcaprolactam/N-vinyl-pyrrolidone/N-vinylimidazolium salts (Luviquat™Hold); cationic cellulose derivatives (Polyquaternium-4 and -10), acrylamido copolymers (Polyquaternium-7) and chitosan. Suitable cationic (quaternized) polymers are also Merquat™ (polymer based on dimethyldiallylammonium chloride), Gafquat™ (quaternary polymers which form by the reaction of polyvinylpyrrolidone with quaternary ammonium compounds), polymer™ JR (hydroxyethylcellulose with cationic groups) and plant-based cationic polymers, e.g. guar polymers, such as the Jaguar™ grades from Rhodia.

Suitable additional polymers are also amphoteric or zwitterionic polymers, such as the octylacrylamide/methyl methacrylate/tert-butylaminoethyl methacrylate/2-hydroxypropyl methacrylate copolymers obtainable under the names Amphomer™ (National Starch) and zwitterionic polymers, as are described, for example, in the German patent applications DE 39 29 973, DE 21 50 557, DE 28 17 369 and DE 37 08 451. Acrylamidopropyltrimethylammonium chloride/acrylic acid or methacrylic acid copolymers and the alkali metal and ammonium salts thereof are preferred zwitterionic polymers. Further suitable zwitterionic polymers are methacroylethylbetaine/methacrylate copolymers which are commercially available under the name Amersette™ (AMERCHOL), and copolymers of hydroxyethyl methacrylate, methyl methacrylate, N,N-dimethylaminoethyl methacrylate and acrylic acid (Jordapon™).

Neutral polymers suitable as additional polymers are, for example, polyvinylpyrrolidones, copolymers of N-vinylpyrrolidone and vinyl acetate and/or vinyl propionate, polysiloxanes, polyvinylcaprolactam and other copolymers with N-vinyl-pyrrolidone, polyethyleneimines and salts thereof, polyvinylamines and salts thereof, cellulose derivatives, polyaspartic acid salts and derivatives. These include, for example, Luviflex™ Swing (partially saponified copolymer of polyvinyl acetate and polyethylene glycol, BASF).

Suitable polymers are also nonionic, water-soluble or water-dispersible polymers or oligomers, such as polyvinylcaprolactam, e.g. Luviskol™Plus (BASF), or polyvinylpyrrolidone and copolymers thereof, in particular with vinylesters, such as vinyl acetate, e.g. Luviskol™VA 37 (BASF); polyamides, e.g. based on itaconic acid and aliphatic diamines, as are described, for example, in DE-A-43 33 238.

Suitable polymers are also nonionic, siloxane-containing, water-soluble or -dispersible polymers, e.g. polyethersiloxanes, such as Tegopren™ (Goldschmidt) or Belsil™ (Wacker).

The polymers suitable for the use according to the invention can also be used for modifying the rheology of skin-cleansing compositions.

Skin-cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, showering and bathing preparations, such as washing lotions, shower baths and gels, foam baths, oil baths and scrub preparations, shaving foams, lotions and creams.

The polymers suitable for use according to the invention can also be used for modifying the rheology of cosmetic compositions for the care and protection of the skin, nail care compositions or preparations for decorative cosmetics.

Such skin cosmetic compositions are, for example, face toners, face masks, deodorants and other cosmetic lotions. Compositions for use in decorative cosmetics comprise, for example, concealing sticks, stage make-up, mascara and eye shadows, lipsticks, kohl pencils, eyeliners, blushers, powder and eyebrow pencils.

Furthermore, the polymers suitable for the use according to the invention can be used in nose strips for pore cleansing, in antiacne compositions, repellents, shaving compositions, hair-removal compositions, intimate care compositions, foot care compositions, and in baby care.

The skin care compositions are, in particular, W/O or O/W skin creams, day and night creams, eye creams, face creams, antiwrinkle creams, moisturizing creams, bleach creams, vitamin creams, skin lotions, care lotions and moisturizing lotions.

Skin cosmetic and dermatological compositions comprise preferably 0.05 to 20% by weight, preferably 0.1 to 15% by weight, very particularly preferably 0.1 to 10% by weight, of the polymers suitable for the use according to the invention, based on the total weight of the composition.

Particularly photoprotective agents for whose rheology modification the polymers suitable for the use according to the invention are used have the property of increasing the residence time of the UV-absorbing ingredients compared to customary auxiliaries such as polyvinylpyrrolidone.

Depending on the field of use, the compositions can be applied in a form suitable for skin care, such as, for example, in the form of a cream, foam, gel, stick, mousse, milk, spray (pump spray or propellant-containing spray) or lotion.

Besides the rheology-modifying polymer and suitable carriers, the skin cosmetic preparations can also comprise further active ingredients and auxiliaries customary in skin cosmetics and as described above. These include preferably emulsifiers, preservatives, perfume oils, cosmetic active ingredients, such as phytantriol, vitamin A, E and C, retinol, bisabolol, panthenol, photoprotective agents, bleaches, colorants, tinting agents, tanning agents, collagen, protein hydrolysates, stabilizers, pH regulators, dyes, salts, other thickeners, gel formers, consistency regulators, silicones, humectants, refatting agents and further customary additives.

Preferred oil and fat components of the skin cosmetic and dermatological compositions are the abovementioned mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanoline, or waxes, fatty acids, fatty acid esters, such as, for example, triglycerides of C₆-C₃₀-fatty acids, wax esters, such as, for example, jojoba oil, fatty alcohols, Vaseline, hydrogenated lanoline and acetylated lanoline, and mixtures thereof.

To establish certain properties, such as, for example, improving the feel to the touch, the spreading behavior, the water resistance and/or the binding of active ingredients and auxiliaries, such as pigments, the skin cosmetic and dermatological preparations can additionally also comprise conditioning substances based on silicone compounds. Suitable silicone compounds are, for example, polyalkylsiloxanes, polyarylsiloxanes, polyarylalkylsiloxanes, polyethersiloxanes or silicone resins.

The cosmetic or dermatological preparations are produced by customary methods known to the person skilled in the art.

Preferably, the cosmetic and dermatological compositions are in the form of emulsions, in particular water-in-oil (W/O) or oil-in-water (O/W) emulsions. However, it is also possible to choose other types of formulation, for example hydrodispersions, gels, oils, oleogels, multiple emulsions, for example in the form of W/O/W or O/W/O emulsions, anhydrous ointments or ointment bases, etc.

Emulsions are produced by known methods. Besides at least one polymer suitable for the use according to the invention, the emulsions usually comprise customary constituents, such as fatty alcohols, fatty acid esters and, in particular, fatty acid triglycerides, fatty acids, lanoline and derivatives thereof, natural or synthetic oils or waxes and emulsifiers in the presence of water. The selection of the additives specific to the type of emulsion and the preparation of suitable emulsions is described, for example, in Schrader, Grundlagen und Rezepturen der Kosmetika [Fundamentals and formulations of cosmetics], Hüthig Buch Verlag, Heidelberg, 2nd Edition, 1989, third part, which is hereby expressly incorporated by reference.

A suitable emulsion, e.g. for a skin cream etc., generally comprises an aqueous phase which has been emulsified by means of a suitable emulsifier system in an oil or fatty phase.

Preferred fatty components which may be present in the fatty phase of the emulsions are: hydrocarbon oils, such as paraffin oil, purcellin oil, perhydrosqualene and solutions of microcrystalline waxes in these oils; animal or vegetable oils, such as sweet almond oil, avocado oil, calophylum oil, lanoline and derivatives thereof, castor oil, sesame oil, olive oil, jojoba oil, karité oil, hoplostethus oil; mineral oils whose distillation starting point under atmospheric pressure is at about 250° C. and whose distillation end point is at 410° C., such as, for example, Vaseline oil; esters of saturated or unsaturated fatty acids, such as alkyl myristates, e.g. i-propyl, butyl or cetyl myristate, hexadecyl stearate, ethyl or isopropyl palmitate, octanoic or decanoic acid triglycerides and cetyl ricinoleate.

The fatty phase can also comprise silicon oils soluble in other oils, such as dimethylpolysiloxane, methylphenylpolysiloxane and the silicone glycol copolymer, fatty acids and fatty alcohols.

In addition, it is also possible to use waxes, such as, for example, carnauba wax, candililla wax, beeswax, microcrystalline wax, ozocerite wax and Ca, Mg and Al oleates, myristates, linoleates and stearates.

In addition, an emulsion may be in the form of a O/W emulsion. Such an emulsion usually comprises an oil phase, emulsifiers which stabilize the oil phase in the water phase, and an aqueous phase which is usually present in thickened form. Suitable emulsifiers are preferably O/W emulsifiers, such as polyglycerol esters, sorbitan esters or partially esterified glycerides.

According to a further preferred embodiment, the rheology-modifying polymers are particularly advantageously used in shower gels, shampoo formulations or bath preparations.

Furthermore, such formulations usually comprise anionic surfactants as base surfactants and amphoteric and/or nonionic surfactants as cosurfactants. Further suitable active ingredients and/or auxiliaries are generally chosen from lipids, perfume oils, fat dyes, organic acids, preservatives and antioxidants, and also thickeners/gel formers, skin conditioners and moisturizers.

These formulations advantageously comprise 2 to 50% by weight, preferably 5 to 40% by weight, particularly preferably 8 to 30% by weight, of surfactants, based on the total weight of the formulation.

In the washing, showering and bathing preparation it is possible to use all of the anionic, neutral, amphoteric or cationic surfactants customarily used in body-cleansing compositions.

Suitable anionic surfactants are, for example, alkyl sulfates, alkyl ether sulfates, alkylsulfonates, alkylarylsulfonates, alkyl succinates, alkylsulfosuccinates, N-alkyl sarcosinates, acyl taurates, acyl isothionates, alkyl phosphates, alkyl ether phosphates, alkyl ether carboxylates, alpha-olefinsulfonates, in particular the alkali metal and alkali earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide unit, preferably 1 to 3 ethylene oxide units, in the molecule.

These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkylamphoacetates or amphopropionates, alkyl amphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mols per mole of alcohol. In addition, alkylamine oxides, mono- or dialkylalkanolamides, fatty acid esters of polyethylene glycols, ethoxylated fatty acid amides, alkyl polyglycosides or sorbitan ether esters are suitable.

In addition, the washing, showering and bathing preparations can comprise customary cationic surfactants, such as, for example, quaternary ammonium compounds, for example cetyltrimethylammonium chloride.

In addition, the shower gel/shampoo formulations can comprise further thickeners, such as, for example, sodium chloride, PEG-55, propylene glycol oleate, PEG-120 methylglucose dioleate and others, and also preservatives, further active ingredients and auxiliaries and water.

A particularly preferred embodiment of the invention is hair-treatment compositions.

Hair-treatment compositions preferably comprise a polymer suitable for the use according to the invention in an amount in the range from about 0.1 to 20% by weight, preferably 0.3 to 15% by weight, based on the total weight of the composition.

Preferably, the hair-treatment compositions according to the invention are in the form of a setting foam, hair mousse, hair gel, shampoo, hairspray, hair foam, end fluids, neutralizers for permanent waves, hair colorants and bleaches or hot-oil treatments. Depending on the field of use, the hair cosmetic preparations can be applied in the form of an (aerosol) spray, (aerosol) foam, gel, gel spray, cream, lotion or wax. Here, hairsprays comprise both aerosol sprays and also pump sprays without propellant gas. Hair foams comprise both aerosol foams and also pump foams without propellant gas. Hairsprays and hair foams comprise preferably predominantly or exclusively water-soluble or water-dispersible components. If the compounds used in the hairsprays and hair foams according to the invention are water-dispersible, they can be used in the form of aqueous microdispersions with particle diameters of from usually 1 to 350 nm, preferably 1 to 250 nm. The solids contents of these preparations here are usually in a range from about 0.5 to 20% by weight. These microdispersions generally require no emulsifiers or surfactants for their stabilization.

In a preferred embodiment of the invention, the compositions according to the invention comprise a fraction of volatile organic components (VOCs) of at most 80% by weight, particularly preferably at most 55% by weight.

The hair cosmetic formulations according to the invention comprise, in a preferred embodiment,
a) 0.05 to 20% by weight of at least one polymer suitable for the use according to the invention,
b) 20 to 99.95% by weight of water and/or alcohol,
c) 0 to 50% by weight of at least one propellant gas,
d) 0 to 5% by weight of at least one emulsifier,
e) up to 25% by weight of further constituents.

Alcohol is understood as meaning all alcohols customary in cosmetics, e.g. ethanol, isopropanol, n-propanol.

Further constituents are understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein hydrolysates, alpha- and beta-hydroxycarboxylic acids, protein hydrolysates, stabilizers, pH regulators, dyes, viscosity regulators, gel formers, dyes, salts, humectants, refatting agents, complexing agents and further customary additives.

All ingredients suitable for cosmetic compositions may, if appropriate, also be used for the hair cosmetic compositions. These also include all styling, setting and conditioning polymers known in cosmetics.

To establish certain properties, the preparations can additionally also comprise conditioning substances based on silicone compounds. Suitable silicone compounds are, for example, polyalkylsiloxanes, polyarylsiloxanes, polyarylalkylsiloxanes, polyethersiloxanes, silicone resins or dimethicone copolyols (CTFA) and aminofunctional silicone compounds, such as amodimethicone (CTFA).

Emulsifiers which may be used are all emulsifiers customarily used in hair foams. Suitable emulsifiers may be nonionic, cationic or anionic or amphoteric.

Propellants which are particularly suitable for aerosol foams are mixtures of dimethyl ether and, if appropriate halogenated, hydrocarbons, such as propane, butane, pentane or HFC-152 a. In this case the ratios of the propellants are to be varied depending on further solvents and desired application.

Examples of nonionic emulsifiers (INCl nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, quaternium-1 to x (INCl).

Anionic emulsifiers can, for example, be chosen from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:
a) 0.1 to 10% by weight of at least one polymer suitable for the use according to the invention,
b) 80 to 99.85% by weight of water and/or alcohol,
c) 0 to 20% by weight of further constituents.

The use of the polymers suitable for the use according to the invention as gel formers is advantageous if specific rheological or other application-related properties of the gels are to be established. On account of the excellent compatibility with the polymers suitable for the use according to the invention, it is also possible to use further gel formers customary in cosmetics. These include slightly crosslinked polyacrylic acid, for example carbomer (INCl), cellulose derivatives, e.g. hydroxypropylcellulose, hydroxyethylcellulose, cationically modified celluloses, polysaccharides, e.g. xanthan gum, caprylic/capric triglyceride, sodium acrylate copolymers, polyquaternium-32 (and) paraffinum liquidum (INCl), sodium acrylate copolymers (and) paraffinum liquidum (and) PPG-1 trideceth-6, acrylamidopropyltrimonium chloride/acrylamide copolymers, steareth-10 allyl ether acrylate copolymers, polyquaternium-37 (and) paraffinum liquidum (and) PPG-1 trideceth-6, polyquaternium 37 (and) propylene glycol dicaprate dicaprylate (and) PPG-1 trideceth-6, polyquaternium-7, polyquaternium-44.

The polymers can be used according to the invention as thickeners in shampoos. Preferred shampoo formulations comprise
a) 0.05 to 10% by weight of at least one polymer suitable for the use according to the invention,
b) 25 to 94.95% by weight of water,
c) 5 to 50% by weight of surfactant,
d) 0 to 5% by weight of a further conditioning agent,
e) 0 to 10% by weight of further cosmetic constituents.

Polymers containing methacrylamide as component b) incorporated during the polymerization are particularly suitable for use as thickeners in shampoos and other compositions containing surfactants.

In the shampoo formulations, it is possible to use all of the anionic, neutral, amphoteric or cationic surfactants customarily used in shampoos.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

For example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate are suitable.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mols per mole of alcohol. In addition, alkylamine oxides, mono- or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethylammonium chloride.

To achieve certain effects, customary conditioners may be used in the shampoo formulations. These include, for example, the abovementioned cationic polymers with the INCl name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamide copolymers (Polyquaternium-7). In addition, it is possible to use protein hydrolysates, and conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and aminofunctional silicone compounds, such as amodimethicones (CTFA). It is also possible to use cationic guar derivatives, such as guar hydroxypropyltrimonium chloride (INCl).

The invention also provides aqueous compositions containing at least one polymer suitable for the use according to the invention, and polyvinylpyrrolidone, suitable polyvinylpyrrolidones being those with a K value of at least 30, preferably of at least 60 and particularly preferably of at least 90. Such polyvinylpyrrolidones are commercially available e.g. under the trade name Luviskol™ (BASF). Even at low concentrations of polymer suitable for the use according to the invention, and of polyvinylpyrrolidone [0.5% by weight], such compositions especially as gels, surprisingly have very good properties, such as a high viscosity and clarity of the aqueous composition and a very good strengthening action when applied to the hair.

### EXAMPLES

The Examples which follow will illustrate the invention in detail without implying a limitation.

### Preparation of the Polymers Suitable for the use According to the Invention

The initial ingredients were heated to 90° C. under nitrogen in a stirred reactor equipped with a nitrogen inlet, a reflux condenser and a metering device. Addition 1 was then metered in over 4 hours and Addition 2 over 5 hours (⅓ in 3 hours, ⅔ in 2 hours). When Addition 2 had ended, the mixture was heated to 100° C. and polymerization was continued for a further 2 hours at this temperature. The mixture was then cooled to room temperature (approx. 23° C.) and diluted with Addition 3 and the precipitated polymer was filtered off, washed with acetone, dried under suction and dried at 75° C. in a vacuum drying cabinet (water-jet pump vacuum).

Abbreviations used:

n-BuAc: n-butyl acetate

PEG: polyethylene glycol

VI: N-vinylimidazole

QVI: N-vinylimidazoliummethyl chloride (VI quaternized with methyl chloride)

VP: N-vinylpyrrolidone

VFA: N-vinylformamide

V-Cap: N-vinylcaprolactam

AA: acrylic acid

MAM: methacrylamide

PIB: polyisobutene

PETAE: pentaerythrityl triallyl ether

C18VE: octadecyl vinyl ether

C18 AC: stearyl methacrylate

C22AC: behenyl acrylate

Unless indicated otherwise, the amounts indicated in the Table below are in grams [g] and the ratios are by weight (w/w).

| | Polymer | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | 13 |
| Initial ingredients | | | | | | | | | | | | | |
| n-BuAc [g] | 350 | 350 | 350 | 350 | 350 | 350 | 350 | 350 | 350 | 350 | 350 | 350 | 350 |
| M_{w} PEG | — | 35 | 35 | 35 | 35 | 35 | 9 | 9 | 9 | 9 | 35 | 35 | 35 |
| [10³ g/mol] | | | | | | | | | | | | | |
| Component | — | 49 | 35 | 70 | 87.5 | 105 | 70 | 70 | 70 | 70 | 70 | 70 | 140 |
| f) PEG [g] | | | | | | | | | | | | | |
| Component | 0.90 | 0.90 | 1.10 | 0.90 | 0.84 | 0.78 | 0.90 | 1.26 | 1.79 | 0.90 | 0.90 | 0.90 | 1.41 |
| c) PETAE [g] | | | | | | | | | | | | | |
| Addition 2 [g] | 14 | 14 | 14 | 14 | 14 | 14 | 14 | 14 | 14 | 14 | 14 | 14 | 14 |
| Addition 1: | | | | | | | | | | | | | |
| n-BuAc [g] | 262 | 262 | 262 | 262 | 262 | 262 | 262 | 262 | 262 | 262 | 262 | 262 | 262 |
| Component | 168 | 118 | 189 | 168 | 158 | 147 | 168 | 168 | 168 | — | — | — | 70 |
| b) VP [g] | | | | | | | | | | | | | |
| Component | 112 | 118 | 126 | 112 | 105 | 98 | 112 | 112 | 112 | 280 | 112 | 112 | 140 |
| a) VI [g] | | | | | | | | | | | | | |
| Component | — | — | — | — | — | — | — | — | — | — | VFA | V-Cap | |
| b) [g] | | | | | | | | | | | 168 | 168 | |
| Component | | | | | | | | | | | | | MMA |
| e) [g] | | | | | | | | | | | | | 70 |
| Ratio | 60/40 | 50/50 | 60/40 | 60/40 | 60/40 | 60/40 | 60/40 | 60/40 | 60/40 | 0/100 | 60/40 | 60/40 | 33/66 |
| VP/VI (w/w) | | | | | | | | | | | VFA/ | V-Cap/ | |
| | | | | | | | | | | | VI | VI | |
| Addition 2: | | | | | | | | | | | | | |
| n-BuAc [g] | 310 | 310 | 310 | 310 | 310 | 310 | 310 | 310 | 310 | 310 | 310 | 310 | 310 |
| Initiator* | 0.56 | 0.56 | 0.56 | 0.56 | 0.56 | 0.56 | 0.56 | 0.56 | 0.56 | 0.56 | 0.56 | 0.56 | 0.56 |
| Addition 3: | | | | | | | | | | | | | |
| n-BuAc [g] | 37 | 37 | 37 | 37 | 37 | 37 | 37 | 37 | 37 | 37 | 37 | 37 | 37 |
| Initiator* | 3.23 | 3.23 | 3.23 | 3.23 | 3.23 | 3.23 | 3.23 | 3.23 | 3.23 | 3.23 | 3.23 | 3.23 | 3.23 |
| Addition 4: | | | | | | | | | | | | | |
| Acetone [g] | 450 | 450 | 450 | 450 | 450 | 450 | 450 | 450 | 450 | 450 | 450 | 450 | 450 |
| Visco 1** | 21.4 | 19.8 | 14.4 | 32.0 | 30.9 | 6.0 | 19.8 | 86.0 | 85.0 | 90.0 | 350.0 | 8.4 | 32.0 |
| Visco 0.5** | 15.9 | 11.9 | 15.0 | 18.6 | 17.3 | 3.1 | 9.0 | 24.5 | 50.0 | 28.4 | 37.3 | 5.5 | 13.8 |
| Degree of | 90 | 85 | 97 | 91 | 94 | 98 | 96 | 98 | 93 | 80 | 94 | 95 | 85 |
| alkylation [%] | | | | | | | | | | | | | |
| | Polymer | | | | | | | | | | | | |
| | 14 | 15 | 16 | 17 | 18 | 19 | 20 | 21 | 22 | 23 | 24 | 25 | 26 |
| Initial | | | | | | | | | | | | | |
| ingredients: | | | | | | | | | | | | | |
| n-BuAc [g] | 350 | 350 | 350 | 350 | 350 | 350 | 350 | 350 | 350 | 350 | 350 | 350 | 350 |
| M_{w} PEG | — | — | | — | — | 9 | 9 | 9 | 9 | — | — | — | — |
| [10³ g/mol] | | | | | | | | | | | | | |
| Component f) PEG [g] | — | — | — | — | — | 15 | 30 | 70 | 70 | — | — | — | — |
| Component c) | 1.26 | 1.79 | 1.79 | 2.1 | 2.1 | 1.26 | 1.26 | 1.79 | 2.1 | 1.79 | 1.79 | 1.79 | 2.1 |
| PETAE [g] | | | | | | | | | | | | | |
| Addition 2 [g] | 14 | 14 | 14 | 14 | 14 | 14 | 14 | 14 | 14 | 14 | 14 | 14 | 14 |
| Addition 1: | | | | | | | | | | | | | |
| n-BuAc [g] | 262 | 262 | 262 | 262 | 262 | 262 | 262 | 262 | 262 | 262 | 262 | 262 | 262 |
| Component b) | — | 112 | — | 112 | — | — | | | 60 | — | — | 60 | 60 |
| VP [g] | | | | | | | | | | | | | |
| Component a) | 280 | 168 | 280 | 168 | 280 | 280 | 280 | 280 | 220 | 252 | 252 | 192 | 220 |
| VI [g] | | | | | | | | | | | | | |
| Component d) | | | | | | | | | | C18VE | C18AC | C18VE | — |
| [g] | | | | | | | | | | 28 | 28 | 28 | |
| Ratio VP/VI (w/w) | 0/100 | 39/61 | 0/100 | 39/61 | 0/100 | 0/100 | 0/100 | 0/100 | 25/75 | 0/100 | 0/100 | 24/76 | 21/79 |
| Addition 2: | | | | | | | | | | | | | |
| n-BuAc [g] | 310 | 310 | 310 | 310 | 310 | 310 | 310 | 310 | 310 | 310 | 310 | 310 | 310 |
| Initiator* | 0.56 | 0.56 | 0.56 | 0.56 | 0.56 | 0.56 | 0.56 | 0.56 | 0.56 | 0.56 | 0.56 | 0.56 | 0.56 |
| Addition 3: | | | | | | | | | | | | | |
| n-BuAc [g] | 37 | 37 | 37 | 37 | 37 | 37 | 37 | 37 | 37 | 37 | 37 | 37 | 37 |
| Initiator* [g] | 3.23 | 3.23 | 3.23 | 323 | 3.23 | 3.23 | 3.23 | 3.23 | 3.23 | 3.23 | 3.23 | 3.23 | 3.23 |
| Addition 4: | | | | | | | | | | | | | |
| Acetone [g] | 450 | 450 | 450 | 450 | 450 | 450 | 450 | 450 | 450 | 450 | 450 | 450 | 450 |
| Visco 1** | 52 | 43 | 41 | 39 | 48 | 45 | 40 | 50 | 38 | 52 | 45 | 43 | 49 |
| Visco 0.5** | 23 | 26 | 31 | 27 | 32 | 29 | 25 | 34 | 25 | 35 | 32 | 34 | 32 |
| Degree of | 48 | 43 | 51 | 48 | 55 | 32 | 31 | 45 | 57 | 40 | 70 | 66.5 | 35 |
| alkylation*** | | | | | | | | | | | | | |
| | Polymer | | | | | | | | | | | | |
| | 27 | 28 | 29 | 30 | 31 | 32 | 33 | 34 | 35 | 36 | 37 | 38 | 39 |
| Initial | | | | | | | | | | | | | |
| ingredients: | | | | | | | | | | | | | |
| n-BuAc [g] | 350 | 350 | 350 | 350 | 350 | 350 | 350 | 350 | 350 | 350 | 350 | 350 | 350 |
| M_{w} PEG | — | — | 9 | — | 9 | 9 | — | 9 | 9 | — | — | 9 | — |
| [10³ g/mol] | | | | | | | | | | | | | |
| Component f) | — | — | 70 | — | 35 | 35 | — | 70 | 35 | — | — | 70 | — |
| PEG [g] | | | | | | | | | | | | | |
| Component | 1.58 | 2.24 | 1.26 | 1.58 | 2.24 | 1.58 | 1.05 | 1.58 | 2.24 | 1.68 | 1.58 | 1.73 | 2.1 |
| c) PETAE [g] | | | | | | | | | | | | | |
| Addition 2 [g] | 14 | 14 | 14 | 14 | 14 | 14 | 14 | 14 | 14 | 14 | 14 | 14 | 14 |
| Addition 1: | | | | | | | | | | | | | |
| n-BuAc [g] | 262 | 262 | 262 | 262 | 262 | 262 | 262 | 262 | 262 | 262 | 262 | 262 | 262 |
| Component | — | — | — | — | 52.5 | 52.5 | 227 | 70 | 52.5 | 87.5 | — | 70 | 227 |
| b) VP [g] | | | | | | | | | | | | | |
| Component | 315 | 332 | 280 | 315 | 245 | 245 | 78 | 140 | 245 | 245 | 280 | 140 | 78 |
| a) VI [g] | | | | | | | | | | | | | |
| Component | C18VE | C18VE | — | C22AC | — | C18AC | — | C18AC | — | — | | C18AC | — |
| d) [g] | 35 | 17.5 | | 35 | | 17.5 | | 35 | | | | 35 | |
| Component | — | — | — | — | — | — | MAM | — | — | MAM | — | | MAM |
| b) [g] | | | | | | | 43 | | | 17.5 | | | 43 |
| Lutensol ® AT- | | | | | 17.5 | | | 35 | 17.5 | — | 70 | 35^{#} | |
| 25 (meth)- | | | | | | | | | | | | | |
| acrylate [g] | | | | | | | | | | | | | |
| Component | | | | | | | | | | | | MMA | |
| e) [g] | | | | | | | | | | | | 105 | |
| Ratio VP/VI | 0/100 | 0/100 | 0/100 | 0/100 | 17/83 | 17/83 | 74/26 | 33/67 | 17/83 | 26/74 | 0/100 | 33/67 | 74/26 |
| (w/w) | | | | | | | | | | | | | |
| Addition 2: | | | | | | | | | | | | | |
| n-BuAc [g] | 310 | 310 | 310 | 310 | 310 | 310 | 310 | 310 | 310 | 310 | 310 | 310 | 310 |
| Initiator* [g] | 0.56 | 0.56 | 0.56 | 0.56 | 0.56 | 0.56 | 0.56 | 0.56 | 0.56 | 0.56 | 0.56 | 0.62 | 0.56 |
| Addition 3: | | | | | | | | | | | | | |
| n-BuAc [g] | 37 | 37 | 37 | 37 | 37 | 37 | 37 | 37 | 37 | 37 | 37 | 37 | 37 |
| Initiator* [g] | 3.23 | 3.23 | 3.23 | 3.23 | 3.23 | 3.23 | 3.23 | 3.23 | 3.23 | 3.23 | 3.23 | 3.23 | 3.23 |
| Addition 4: | | | | | | | | | | | | | |
| Acetone [g] | 450 | 450 | 450 | 450 | 450 | 450 | 450 | 450 | 450 | 450 | 450 | 450 | 450 |
| Visco 2** | | | | | | | 10 | 6 | | | | | 40 |
| Visco 1** | 20.4 | 40 | 35 | 21 | 41 | 19 | 4.9 | 3 | 41 | 34 | 182 | 4 | 16 |
| Visco 0.5** | 14.7 | 20 | 15 | 9 | 26 | 7 | 3.2 | 1 | 26 | 25 | 21 | 1 | 1.7 |
| Degree of | 75 | 86 | 43 | 60 | 70 | 65 | 95 | 92 | 75 | 72 | 68 | 86 | 91 |
| alkylation*** | | | | | | | | | | | | | |

| | Polymer | | | | | | | | | |
|---|---|---|---|---|---|---|---|---|---|---|
| | 40 | 41 | 42 | 43 | 44 | 45 | 46 | 47 | 48 | 49 |
| Initial | | | | | | | | | | |
| ingredients: | | | | | | | | | | |
| n-BuAc [g] | 350 | 350 | 350 | 350 | 774 | 774 | 350 | 350 | 350 | 350 |
| VI [g] | | | | | 315 | 315 | | — | | |
| Lutensol ® AT-25 acrylate [g] | | | | | 45 | 45 | | — | | |
| M_{w} PEG [10³ g/mol] | 9 | 9 | — | — | — | 9 | | — | | — |
| Component f) PEG [g] | 35 | 35 | — | — | — | 90 | | — | | — |
| M_{w} Poly-THF [10³ g/mol] | — | — | — | | 2 | | | | | |
| Component f) Poly-THF [g] | — | — | — | | 90 | | | | | |
| Component c) PETAE [g] | 2.02 | 2.02 | 2.24 | 2.24 | 1.8 | 1.8 | 2.24 | — | — | — |
| Initiator | | | | | 0.58 | 0.58 | | | | |
| Addition 2 [g] | 14 | 14 | 14 | 14 | — | — | 14 | 14 | 14 | 14 |
| Addition 1: | | | | | | | | | | |
| n-BuAc [g] | 262 | 262 | 262 | 262 | 200 | 200 | 262 | 262 | 262 | 262 |
| Component b) VP [g] | — | 52.5 | — | — | | | 110 | | | — |
| Component a) VI [g] | 297 | 245 | 332 | 280 | | | 245 | 315 | 280 | 315 |
| Component d) [g] | C18AC | C18AC | C18AC | — | — | | | C18VE | | C18VE |
| | 17.5 | 17.5 | 17.5 | | | | | 35 | | 35 |
| Component f)/d) Lutensol ® AT- | — | — | — | 70 | | | | | 70 | — |
| 25 acrylate [g] | | | | | | | | | | |
| Component b) [g] | | | | | | | MAM | | | |
| | | | | | | | 35 | | | |
| Component e) | | | | | | | | | | |
| Component c) PETAE [g] | | | | | | | | 2.24 | 2.24 | 1.58 |
| Ratio VP/VI (w/w) | 0/100 | 17/83 | 0/100 | 0/100 | 0/100 | 0/100 | 30/70 | 0/100 | 0/100 | 0/100 |
| Addition 2: | | | | | | | | | | |
| n-BuAc [g] | 310 | 310 | 310 | 310 | | | 310 | 310 | 310 | 310 |
| Initiator* | 0.56 | 0.56 | 0.56 | 0.56 | | | 0.56 | 0.56 | 0.56 | 0.56 |
| Addition 3: | | | | | | | | | | |
| n-BuAc [g] | 37 | 37 | 37 | 37 | 58 | 58 | 37 | 37 | 37 | 37 |
| Initiator* | 3.23 | 3.23 | 3.23 | 3.23 | 3.02 | 3.02 | 3.23 | 3.23 | 3.23 | 3.23 |
| Addition 4: | | | | | | | | | | |
| Acetone [g] | 450 | 450 | 450 | 450 | 450 | 450 | 450 | 450 | 450 | 450 |
| Visco 2** | | | | | | | | | | |
| Visco 1** | 42 | 24 | 180 | 103 | 80 | 30 | 47 | 44 | 50 | 18 |
| Visco 0.5** | 26 | 15 | 43 | 32 | 25 | 23 | 27 | 34 | 30 | 13 |
| Degree of alkylation*** | 66 | 46 | 25 | 53 | 65 | 72 | 68 | 56 | 63 | 70 |

| | Polymer | | | | |
|---|---|---|---|---|---|
| | 50 | 51 | 52 | 53 | 54 |
| Initial ingredients: | | | | | |
| n-BuAc [g] | 350 | 350 | 350 | 350 | 175 |
| M_{w} PEG [10³ g/mol] | 9 | 9 | — | — | 9 |
| Component f) PEG [g] | 35 | 35 | — | — | 17.5 |
| Component f) | 10.5 | 7 | — | 7 | — |
| Belsil ® DMC 6031 | | | | | |
| Component f)/d) | — | — | 35 | — | — |
| Lutensol ® AT-50 [g] | | | | | |
| Component c) PETAE [g] | 1.58 | 1.58 | 1.58 | 1.58 | 0.79 |
| Addition 2 [g] | 14 | 14 | 14 | 14 | 14 |
| Addition 1: | | | | | |
| n-BuAc [g] | 262 | 262 | 262 | 262 | 131 |
| Component b) VP [g] | — | 52.5 | 70 | | — |
| Component a) VI [g] | 315 | 245 | 245 | 315 | 148 |
| Component d) [g] | C18AC | C18AC | — | C18VE | PIB-acrylic acid |
| | 17.5 | 17.5 | | 35 | ester^{&} |
| | | | | | 8.75 |
| Ratio VP/VI (w/w) | 0/100 | 17/83 | 22/88 | 0/100 | 0/100 |
| Addition 2: | | | | | |
| n-BuAc [g] | 310 | 310 | 310 | 310 | 155 |
| Initiator* | 0.56 | 0.56 | 0.56 | 0.57 | 0.28 |
| Addition 3: | | | | | |
| Acetone [g] | 450 | 450 | 450 | 550 | 225 |
| Visco 1** | 185 | 11 | 13 | 32 | 186 |
| Visco 0.5** | 24 | 3.7 | 7 | 23 | 22 |
| Degree of alkylation*** | 71 | 78 | 65 | 62 | 67 |

*tert-Butyl peroctoate was used as initiator.
^{#}Polymer 38 was prepared using Lutensol ® AT-25 methacrylate.
^{&}reaction product of polyisobutene (M_{w} = 550) and acrylic anhydride
**Viscosity: The numerical data are in 1000 mPas. Visco 2: viscosity of a 2% by weight aqueous solution of the polymer 100% neutralized with acid Visco 1: viscosity of a 1% by weight aqueous solution of the polymer 100% neutralized with acid Visco 0.5: viscosity of a 0.5% by weight aqueous solution of the polymer 100% neutralized with acid
To prepare Polymers 33, 36, 39 and 46, VP and MAM were introduced in one addition and VI was introduced separately in a second addition. MAM was added as a solution in VP.
***Derivatives alkylated with methyl chloride were also prepared from all the polymers. This was done by alkylating the polymers in the same solvent directly after the polymerization. These alkylated derivatives of the polymers are denoted by (Q) in the Application Examples below. The polymers were washed with acetone after the polymerization. In the case of the alkylated derivatives, this washing process did not take place until after the alkylation.

Determination of the Curl Retention

Basic Formulation (Aerosol Hairspray):
5% by weight of active substance: test polymer (100% neutralized with AMP)
15% by weight of ethanol
40% by weight of water
40% by weight of dimethyl ether.

The curl retention was determined using swatches weighing approx. 2 g and 15.5 cm in length, taken from fair caucasian human hair.

Treatment of the Swatches:

The swatches were washed twice with aqueous Texapon®NSO solution. They were then rinsed with warm water until foaming was no longer detectable, rinsed again with demineralized water, combed and placed on filter paper to dry.

A water-wave was produced by swelling the swatches for 15 minutes in a solution of ethanol and water (1:1).

The swatch was carefully combed before preparation of the curl. It was fixed to a Plexiglas rod with a rubber band and then combed and wound into a spiral. The curl was firmly fixed with a cotton cloth and rubber band and dried overnight at 70° C. After cooling, the swatches were carefully opened and slipped off the Plexiglas rod without deforming the water-wave. Approx. 1.8 g of the aerosol hairspray prepared as above were uniformly sprayed onto the curl from a distance of 15 cm while the curl was being steadily rotated. The curls were dried horizontally for 1 hour at room temperature. After drying, they were fixed in a holder. Using a ruler, the length L₀ of the curls was recorded and the increase in length was followed during storage in a humidified climate. After storage for 5 h at 25° C. and 90% RH in the climate-controlled chamber, the final length L₁ of the curl was recorded and the curl retention calculated according to the following equation:

$$curl retention in \% = \frac{L - {L}_{t}}{L - {L}_{o}} * 100$$

L=length of hairs (15.5 cm)

L₀=length of hair curl after drying

Lₜ=length of hair curl after climate treatment

The curl retention was given as the mean of the 5 individual measurements.

Determination of the Strengthening Action (Flexural Rigidity):

The strengthening action of the polymers was measured by measurement of the flexural rigidity of thin swatches of hair (each weighing approx. 3 g and 24 cm in length). This was done by immersing the weighed, dry swatches in the polymer solution indicated below (solvent: ethanol/water 55:45 w/w), a uniform wetting of the swatches and distribution of the polymer solution being assured by immersing and removing them three times and then squeezing them between filter paper. The excess solution of film-forming agent was then wiped off between thumb and index finger and the swatches were shaped by hand to give them a round cross section. They were dried overnight in a climate-controlled room at 20° C. and 65% relative humidity. The tests were performed on a tensile tester in the climate-controlled room at 20° C. and 65% relative humidity. The swatch was placed symmetrically at the ends of two cylindrical rollers of the sample holder. Precisely in the middle, the swatch was then bent by approx. 40 mm from above with a rounded die (breaking of the polymer film). The force required (Fmax) was determined with a load cell (50 N). A measured value represents the arithmetic mean of the individual measurements on 5 to 10 identically treated swatches. The values determined were compared with those of a commercially available reference polymer (Amphomer®LV-71) and given in %.

Determination of the Dynamic Viscosity

The viscosities of the solutions of Polymers 1 to 13 were determined in a conventional 150 ml glass beaker using a Brookfield DV-II viscometer under conventional conditions with a no. 4 spindle at 12 rpm.

The viscosities of the solutions of Polymers 14 to 54 were determined in a conventional 250 ml glass beaker using a Brookfield DV-II+ Pro viscometer under conventional conditions with a no. 6 spindle at 20 rpm.

Quaternization of the Polymers

Methylation with methyl chloride:

The methylation with methyl chloride was effected in the conventional manner known to those skilled in the art. For example, the polymer to be quaternized was suspended in n-butyl acetate in a weight ratio of 1:4 and heated in an autoclave. Gaseous methyl chloride was introduced until the desired increase in weight had been achieved.

Alkylation with alkyl bromide:

The alkylation with alkyl bromide was effected in the conventional manner known to those skilled in the art. For example, the amount of alkyl bromide calculated for the desired degree of quaternization, and the same amount of solvent, are added to the polymer suspended in the solvent. The mixture is then heated to the reflux point and stirred for 4 hours under reflux. It is then cooled and, if appropriate, diluted with solvent at the same time, and the product is filtered off with suction, washed, filtered off with suction again and then dried in a vacuum cabinet at 75° C.

Reference Formulation

0.5% by weight of a commercially available polyacrylic acid thickener (Carbopol®940), neutralized with triethanolamine (TEA), is used to formulate a gel which, when applied to the hair, exhibits substantially no conditioning or strengthening effect 3% by weight of a commercially available hair polymer is added to this standard formulation. The application properties are shown in the Table below. The thickening is good, but the other cosmetic properties on the hair are capable of improvement.

### Formulation According to the Invention

0.5% by weight of a polymer suitable for the use according to the invention is used to formulate a gel. 1 or 3% by weight of a commercially available hair polymer is added. The resulting gel formulations are clear and have a good conditioning or strengthening effect, even when the amount of commercially available hair polymer is very small. The application properties are shown in the Table below. The thickening is good and the other cosmetic properties on the hair are markedly superior to the state of the art.

| | | Viscosity | FR** | | | Ease of wet | |
|---|---|---|---|---|---|---|---|
| Product* | Appearance | [mPas] | [cN] | pH | CR*** | combing | Flaking |
| 0.5% Carbopol ® 940 + 3% | clear | 40,700 | 129 +/− 13 | 6.9 | 35 | 21% | little |
| Luviskol ® K90 | | | | | | | |
| 0.5% Polymer 27(Q) + 3% | almost | 35,800 | 235 +/− 36 | 6.9 | 91 | 61% | little |
| Luviskol ® K90 | clear | | | | | | |
| 0.5% Polymer 27(Q) + 1% | clear | 30,900 | 123 +/− 14 | 7.1 | 68 | 57% | none |
| Luviskol ® K90 | | | | | | | |

*ad 100% by weight of water
**FR = flexural rigidity
***CR = curl retention, determined at 25° C. and 90% relative humidity

Thickening action of the polymers suitable for the use according to the invention in cosmetic formulations for the skin (emulsions), compared with Capigel®98 or Ultrez®21

| | Amount | | | | |
|---|---|---|---|---|---|
| | of | | | Viscosity with | Viscosity with |
| | polymer | | | 0.2% by weight | 0.5% by weight |
| | (% by | | Viscosity | of NaCl | of NaCl |
| Polymer | weight) | pH | (mPa * s) | (mPa * s) | (mPa * s) |
| Capigel ® | 0.2 | 7.6 | 21,500 | 6300 | 5400 |
| 98 | | | | | |
| Ultrez ® | 0.2 | 7.8 | 24,000 | 3400 | 2500 |
| 21 | | | | | |
| 30(Q) | 0.2 | 6 | 33,500 | 11,500 | 10,000 |
| 34(Q) | 0.2 | 5.5 | 25,000 | 12,000 | 10,300 |

### Application Examples

I) Cosmetic Formulations for the Hair

The amounts given below are in % by weight, unless expressly indicated otherwise. The amounts of the polymers used according to the invention are given in % by weight of polymer as a solid. When the polymer is used in the form of a solution or dispersion, it is necessary to use the amount of solution or dispersion that corresponds to the amount of polymer required (as indicated in the following Examples). q.s. denotes “quantum satis” and is familiar to those skilled in the art in the field of cosmetic formulations.

A1) Hair Gel

0.5% of polymer of Example 1(Q)

3% of Luviskol™ K90

0.50% of panthenol

q.s. perfume oil

q.s. preservative

ad 100% of water

A2) Hair Gel

0.5% of polymer of Example 1(Q)

2.5% of Luviskol™ K90

0.5% of Luviquat™ Hold

0.50% of panthenol

q.s. perfume oil

q.s. preservative

ad 100% of water

A3) Hair Gel

0.5% of polymer of Example 1(Q)

2.5% of Luviskol™ K90

0.5% of Luviquat™ Supreme

0.50% of panthenol

q.s. perfume oil

q.s. preservative

ad 100% of water

A4) Hair Gel

0.5% of polymer of Example 1(Q)

2.0% of Luviskol™ K90

1% of Luviquat™ Hold

0.50% of panthenol

q.s. perfume oil

q.s. preservative

ad 100% of water

A5) Hair Gel

0.5% of polymer of Example 1(Q)

2.0% of Luviskol™ K90

1% of Luviset™ Clear

0.50% of panthenol

q.s. perfume oil

q.s. preservative

ad 100% of water

A6) Hair gel

0.5% of polymer of Example 1(Q)

2.0% of Luviskol™ K90

1% of Polyquaternium 11

0.50% of panthenol

q.s. perfume oil

q.s. preservative

ad 100% of water

These Examples are repeated with the polymers of Examples 1 to 54 and 2(Q) to 54(Q). Hair gels with very good properties are obtained in each case.

%

A7) Hair strengthening gel

| | |
|---|---|
| 0.5 | polymer of Example 1(Q) |
| 3.0 | Luviskol ™ VA64W |
| 0.20 | panthenol |
| q.s. | perfume oil |
| q.s. | preservative |
| ad 100 | water |

A8) Hair strengthening gel

| | |
|---|---|
| 0.5 | polymer of Example 1(Q) |
| 2.5 | Luviskol ™ VA64W |
| 1.0 | Luviskol ™ K90 |
| 0.20 | panthenol |
| q.s. | perfume oil |
| q.s. | preservative |
| ad 100 | water |

A9) Hair strengthening gel

| | |
|---|---|
| 0.5 | polymer of Example 1(Q) |
| 2.0 | Luviskol ™ VA64W |
| 2.0 | Luviskol ™ K30 |
| 0.20 | panthenol |
| q.s. | perfume oil |
| q.s. | preservative |
| ad 100 | water |

These Examples are repeated with the polymers of Examples 1 to 54 and 2(Q) to 54(Q). Hair strengthening gels with very good properties are obtained in each case.

A10)

| | |
|---|---|
| 2.00 | Polymer 1(Q) |
| 6.00 | Corn Starch Modified (Amaze, National Starch) |
| 0.50 | chitosan |
| q.s. | perfume oil |
| q.s. | PEG-40 Hydrogenated Castor Oil |
| 0.10 | PEG-14 Dimethicone |
| 0.10 | preservative |
| 91.40 | demineralized water |

Preparation: All the components are mixed until a homogeneous mixture is obtained. This Example is repeated with the polymers of Examples 1 to 54 and 2(Q) to 54(Q).

A11)

| | |
|---|---|
| 2.00 | Polymer 1(Q) |
| 5.00 | VP/DMAPA Acrylates Copolymer (Styleze ® CC-10) |
| 84.85 | demineralized water |
| q.s. | perfume oil |
| q.s. | PEG-40 Hydrogenated Castor Oil |
| 0.10 | Dimethicone Copolyol |
| 0.10 | preservative |
| 2.00 | hydroxypropyl cellulose |

Preparation: All the components are mixed until a homogeneous mixture is obtained. This Example is repeated with the polymers of Examples 1 to 54 and 2(Q) to 54(Q).

A12)

| | |
|---|---|
| 2.00 | Polymer 1(Q) |
| 1.00 | VP/Acrylates/Lauryl Methacrylate Copolymer (ISP: Styleze |
| | 2000) |
| 0.26 | aminomethylpropanol |
| 90.64 | demineralized water |
| q.s. | perfume oil |
| q.s. | PEG-40 Hydrogenated Castor Oil |
| 0.10 | sorbitol |
| 0.10 | preservative |
| 2.00 | hydroxypropylguar (Rhodia Inc., N-Hance Hydroxypropylguar) |

Preparation: All the components are mixed until a homogeneous mixture is obtained. This Example is repeated with the polymers of Examples 1 to 54 and 2(Q) to 54(Q).

A13)

| | |
|---|---|
| 6.00 | Polymer 1(Q) |
| 2.00 | Acrylates/C1-2 Succinates/Hydroxyacrylates Copolymer (Rohm & |
| | Haas, Alli LT-120) |
| 0.19 | aminomethylpropanol |
| q.s. | perfume oil |
| q.s. | PEG-40 Hydrogenated Castor Oil |
| 0.10 | PEG-8 |
| 0.10 | preservative |
| 0.50 | hydroxyethyl cellulose |

Preparation: All the components are mixed until a homogeneous mixture is obtained. This Example is repeated with the polymers of Examples 1 to 54 and 2(Q) to 54(Q).

A14)

| | |
|---|---|
| 7.00 | Polymer 1(Q) |
| 7.00 | Methacrylic Acid/Sodium Acrylamidomethyl Propane Sulfonate |
| | Copolymer (Ondeo Nalco, Fixomer A30) |
| 0.70 | triethanolamine |
| q.s. | perfume oil |
| q.s. | PEG-40 Hydrogenated Castor Oil |
| 0.10 | panthenol |
| 0.10 | preservative |
| 84.90 | demineralized water |
| 1.00 | Polyacrylamide/C13-14 Isoparaffin/Laureth-7 (Seppic, |
| | Sepigel 305) |

Preparation. All the components are mixed until a homogeneous mixture is obtained. This Example is repeated with the polymers of Examples 1 to 54 and 2(Q) to 54(Q).

A15)

| | |
|---|---|
| 2.00 | Polymer 1(Q) |
| 1.00 | polyvinylformamide |
| q.s. | perfume oil |
| q.s. | PEG-40 Hydrogenated Castor Oil |
| 0.10 | preservative |
| 0.10 | ethylhexyl methoxycinnamate |
| 0.10 | PEG-14 Dimethicone |

Preparation: All the components are mixed until a homogeneous mixture is obtained. This Example is repeated with the polymers of Examples 1 to 54 and 2(Q) to 54(Q).

A16) Hair conditioning gel, superstrong

| | |
|---|---|
| 1.00 | Polymer 1(Q) |
| 0.50 | Luviquat Hold (Polyquaternium-46) |
| 3.00 | Luviskol VA 64 (VP/VA Copolymer) |
| 0.50 | panthenol |
| 0.50 | Dimethiconol (DC-193, Dow Corning) |
| 0.10 | EDTA |
| q.s. | preservative |
| ad 100 | water |

Preparation: All the components are mixed until a homogeneous mixture is obtained, which is stirred for a further 15 minutes. This Example is repeated with the polymers of Examples 1 to 54 and 2(Q) to 54(Q). A hair conditioning gel with very good properties is obtained in each case.

A17) Gel fluid

| | |
|---|---|
| 3.00 | Kollicoat ® IR (BASF) |
| q.s. | preservative |
| 2.00 | Polymer 1(Q) |
| 1.00 | hydroxypropylguar |
| 5.00 | alcohol |
| 0.20 | niacinamide |
| 0.50 | panthenol |
| 0.50 | Dimethicone Copolyol |
| 0.20 | Amodimethicone |

Preparation: All the components are mixed until a homogeneous mixture is obtained, which is stirred for a further 15 minutes. This Example is repeated with the polymers of Examples 1 to 54 and 2(Q) to 54(Q). A gel fluid with very good properties is obtained in each case.

A18) Hair pudding

| | |
|---|---|
| 3.00 | Kollicoat IR |
| q.s. | preservative |
| 2.00 | Luviset Clear |
| 1.00 | Polymer 1(Q) |
| 0.50 | Dimethicone Copolyol |
| 0.10 | EDTA |
| 0.20 | 4-benzophenone |
| ad 100 | water |

Preparation: All the components are mixed until a homogeneous mixture is obtained, which is stirred for a further 15 minutes. This Example is repeated with the polymers of Examples 1 to 54 and 2(Q) to 54(Q). A hair pudding with very good properties is obtained in each case.

A19) “Wet look” styling gel

| | | |
|---|---|---|
| 0.80 | Polymer 1(Q) | |
| 39.20 | demineralized water | aqua |
| q.s. | Cremophor ® CO 40 | PEG-40 Hydrogenated Castor Oil |
| q.s. | perfume | |
| 55.04 | demineralized water | aqua |
| 4.00 | Luviskol ® K 90 | PVP |
| q.s. | preservative | |

This Example is repeated with the polymers of Examples 1 to 54 and 2(Q) to 54(Q). A “wet look” styling gel with very good properties is obtained in each case.

A20) Hair gel, superstyle, ultrastrong

| | | | |
|---|---|---|---|
| A | 59.40 | demineralized water | aqua |
| | 10.00 | Luviskol ® K 90 Solution | PVP |
| | 8.00 | Luviskol ® VA 64 W | VP/VA Copolymer |
| | 5.00 | Karion F liquid | sorbitol |
| | 0.10 | Edeta ® BD (1) | disodium EDTA |
| | q.s. | perfume | |
| | q.s. | Cremophor ® CO 40 | PEG-40 Hydrogenated Castor |
| | | | Oil |
| | 0.05 | Uvinul ® MS 40 | 4-benzophenone |
| | q.s. | preservative | |
| | 10.00 | ethanol 96% | alcohol |
| | 6.37 | demineralized water | aqua |
| B | 0.30 | Polymer 1(Q) | |

This Example is repeated with the polymers of Examples 1 to 54 and 2(Q) to 54(Q). A hair gel, superstyle, ultrastrong with very good properties is obtained in each case.

%

A21) Hair strengthening gel

| | |
|---|---|
| 0.5 | Polymer 1(Q) |
| 1.0 | Luviskol ™ K90 |
| 0.20 | panthenol |
| q.s. | perfume oil |
| q.s. | preservative |
| ad 100 | water |

A22) Hair strengthening gel

| | |
|---|---|
| 0.3 | Polymer 1(Q) |
| 1.0 | Luviskol ™ K90 |
| 0.20 | panthenol |
| q.s. | perfume oil |
| q.s. | preservative |
| ad 100 | water |

A23) Hair strengthening gel

| | |
|---|---|
| 0.5 | Polymer 1(Q) |
| 1.5 | Luviskol ™ K90 |
| 0.20 | panthenol |
| q.s. | perfume oil |
| q.s. | preservative |
| ad 100 | water |

A24) Hair strengthening gel

| | |
|---|---|
| 0.5 | Polymer 1(Q) |
| 2 | Luviskol ™ K90 |
| 0.20 | panthenol |
| q.s. | perfume oil |
| q.s. | preservative |
| ad 100 | water |

A25) Hair strengthening gel

| | |
|---|---|
| 0.5 | Polymer 1(Q) |
| 2.5 | Luviskol ™ K90 |
| 0.20 | panthenol |
| q.s. | perfume oil |
| q.s. | preservative |
| ad 100 | water |

A26) Hair strengthening gel

| | |
|---|---|
| 0.5 | Polymer 1(Q) |
| 1 | Luviskol ™ K90 |
| 2 | Luviskol ™ K30 |
| 0.20 | panthenol |
| q.s. | perfume oil |
| q.s. | preservative |
| ad 100 | water |

A27) Hair strengthening gel

| | |
|---|---|
| 0.5 | Polymer 1(Q) |
| 2 | Luviskol ™ K90 |
| 1 | Luviskol ™ VA 64 |
| 0.20 | panthenol |
| q.s. | perfume oil |
| q.s. | preservative |
| ad 100 | water |

A28) Hair strengthening gel

| | |
|---|---|
| 2 | Polymer 1(Q) |
| 0.20 | panthenol |
| q.s. | perfume oil |
| q.s. | preservative |
| ad 100 | water |

These Examples are repeated with the polymers of Examples 1 to 54 and 2(Q) to 54(Q). A hair strengthening gel with very good properties is obtained in each case.

%

A29) Hair conditioning gel

| | |
|---|---|
| 1 | Polymer 1(Q) |
| 0.20 | panthenol |
| q.s. | perfume oil |
| q.s. | preservative |
| ad 100 | water |

A30) Hair conditioning gel

| | |
|---|---|
| 1.5 | Polymer 1(Q) |
| 0.20 | panthenol |
| q.s. | perfume oil |
| q.s. | preservative |
| ad 100 | water |

A31) Hair conditioning gel

| | |
|---|---|
| 0.5 | Polymer 1(Q) |
| 0.20 | panthenol |
| q.s. | perfume oil |
| q.s. | preservative |
| ad 100 | water |

These Examples are repeated with the polymers of Examples 1 to 54 and 2(Q) to 54(Q). A hair conditioning gel with very good properties is obtained in each case.

II) Cosmetic Formulations for the Skin

O/W foaming emulsions

| | Emulsion 1 | | Emulsion 2 | |
|---|---|---|---|---|
| | % by | % by | % by | % by |
| | weight | volume | weight | volume |
| Stearic acid | 5.00 | | 1.00 | |
| Cetyl alcohol | 5.50 | | | |
| Cetylstearyl alcohol | | | 2.00 | |
| PEG-40 stearate | 8.50 | | | |
| PEG-20 stearate | | | 1.00 | |
| Caprylic/capric triglyceride | 4.00 | | 2.00 | |
| C12-15 alkyl benzoate | 10.00 | | 15.00 | |
| Cyclomethicone | 4.00 | | | |
| Dimethicone | | | 0.50 | |
| Polymer 1(Q) | 1.00 | | | |
| Octyl isostearate | | | 5.00 | |
| Myristyl myristate | | | 2.00 | |
| Ceresin | 1.50 | | | |
| Glycerol | | | 3.00 | |
| Filter | | | | |
| Hydroxypropyl distarch phosphate | 1.00 | | 3.50 | |
| BHT | | | 0.02 | |
| Disodium EDTA | 0.50 | | 0.10 | |
| Perfume, preservative | q.s. | | q.s. | |
| Dyestuffs | q.s. | | q.s. | |
| Potassium hydroxide | q.s. | | q.s. | |
| Water | ad 100 | | ad 100 | |
| | pH adjusted | | pH adjusted | |
| | to 6.5-7.5 | | to 5.0-6.0 | |
| Emulsion 1 | | 70 | | |
| Emulsion 2 | | | | 35 |
| Gas (nitrogen) | | 30 | | |
| Gas (helium) | | | | 65 |

Analogous formulations are also prepared with Polymers 1 to 54 or the quaternized derivatives 2(Q) to 54(Q).

Gel cream

| | 1 | 2 | 3 | 4 |
|---|---|---|---|---|
| Acrylate/C10-30 alkyl acrylate crosspolymer | 0 | 0 | 0.40 | 0 |
| Polyacrylic acid | 0.20 | 0 | 0 | 0 |
| Polymer 1(Q) | 0.50 | 0.95 | 1.10 | 0.7 |
| Cetearyl alcohol | 3.00 | 2.50 | 3.00 | 2.50 |
| C12-15 alkyl benzoate | 4.00 | 4.50 | 4.00 | 4.50 |
| Caprylic/capric triglyceride | 3.00 | 3.50 | 3.00 | 3.50 |
| Aminobenzophenone | 2.00 | 1.50 | 0.75 | 1.00 |
| UVASorb K2A | | 3.00 | | |
| Ethylhexyl methoxycinnamate | 3.00 | | 1.00 | |
| Bis-ethylhexyloxyphenol methoxyphenyl triazine | | 1.50 | | 2.00 |
| Butyl methoxydibenzoylmethane | | | 2.00 | |
| Disodium phenyl dibenzimidazole tetrasulfonate | 2.50 | | 0.50 | 2.00 |
| Ethylhexyl triazone | 4.00 | | 3.00 | 4.00 |
| Octocrylene | | 4.00 | | |
| Diethylhexyl butamido triazone | 1.00 | | | 2.00 |
| Phenylbenzimidazole sulfonic acid | 0.50 | | 3.00 | |
| Methylene bis-benzotriazolyl | 2.00 | | 0.50 | 1.50 |
| tetramethylbutylphenol | | | | |
| Ethylhexyl salicylate | | | 3.00 | |
| Drometrizole trisiloxane | | | 0.50 | |
| Terephthalidene dicamphor sulfonic acid | | 1.50 | | 1.00 |
| Diethylhexyl 2,6-naphthalate | 3.50 | 4.00 | 7.00 | 9.00 |
| Titanium dioxide - microfine | 1.00 | | 3.00 | |
| Zinc oxide - microfine | | | | 0.25 |
| Cyclic dimethylpolysiloxane | 5.00 | 5.50 | 5.00 | 5.50 |
| Dimethicone polydimethylsiloxane | 1.00 | 0.60 | 1.00 | 0.60 |
| Glycerol | 1.00 | 1.20 | 1.00 | 1.20 |
| Sodium hydroxide | q.s. | q.s. | q.s. | q.s. |
| Preservative | 0.30 | 0.23 | 0.30 | 0.23 |
| Perfume | 0.20 | | 0.20 | |
| Water | ad 100 | ad 100 | ad 100 | ad 100 |
| pH adjusted to 6.0 | | | | |

Analogous formulations are also prepared with Polymers 1 to 54 or the quaternized derivatives 2(Q) to 54(Q).

O/W sunscreen formulation

| | 1 | 2 | 3 | 4 | 5 | 6 | 7 |
|---|---|---|---|---|---|---|---|
| Glycerol monostearate SE | 0.50 | 1.00 | 3.00 | | | 1.50 | |
| Glyceryl stearate citrate | 2.00 | | 1.00 | 2.00 | 4.00 | | |
| Stearic acid | | 3.00 | | 2.00 | | | |
| PEG-40 stearate | 0.50 | | | | | 2.00 | |
| Cetyl phosphate | | | | | | 1.00 | |
| Cetearyl sulfate | | | | | | | 0.75 |
| Stearyl alcohol | | | 3.00 | | | 2.00 | 0.60 |
| Cetyl alcohol | 2.50 | 1.10 | | 1.50 | 0.60 | | 2.00 |
| Polymer 1(Q) | 2.00 | 1.50 | 1.70 | 0.9 | 1.2 | 1.9 | 1.00 |
| Aminobenzophenone | 2.00 | 1.50 | 0.75 | 1.00 | 2.10 | 4.50 | 5.00 |
| UVASorb K2A | | | | | | | |
| Ethylhexyl | | | | | 5.00 | 6.00 | 8.00 |
| methoxycinnamate | | | | | | | |
| Bis-ethylhexyloxyphenol | | 1.50 | | 2.00 | 2.50 | | 2.50 |
| methoxyphenyl triazine | | | | | | | |
| Butyl | | | 2.00 | | 2.00 | 1.50 | |
| methoxydibenzoylmethane | | | | | | | |
| Disodium phenyl | 2.50 | | 0.50 | 2.00 | | 0.30 | |
| dibenzimidazole | | | | | | | |
| tetrasulfonate | | | | | | | |
| Ethylhexyl triazone | 4.00 | | 3.00 | 4.00 | | 2.00 | |
| Octocrylene | | 4.00 | | | | | 7.50 |
| Diethylhexyl butamido | 1.00 | | | 2.00 | 1.00 | | 1.00 |
| triazone | | | | | | | |
| Phenylbenzimidazole | 0.50 | | 3.00 | | | | |
| sulfonic acid | | | | | | | |
| Methylene bis-benzotriazolyl | 2.00 | | 0.50 | 1.50 | 2.50 | | |
| tetramethylbutylphenol | | | | | | | |
| Ethylhexyl salicylate | | | 3.00 | | | | 5.00 |
| Drometrizole trisiloxane | | | 0.50 | | | 1.00 | |
| Terephthalidene dicamphor | | 1.50 | | 1.00 | 1.00 | | 0.50 |
| sulfonic acid | | | | | | | |
| Diethylhexyl 2,6-naphthalate | 3.50 | | 7.00 | | 6.00 | 9.00 | |
| Titanium dioxide - microfine | 1.00 | | 3.00 | | 3.50 | | 1.50 |
| Zinc oxide - microfine | | | | 0.25 | | 2.00 | |
| C12-15 alkyl benzoate | | 0.25 | | | 4.00 | 7.00 | |
| Dicapryl ether | | | 3.50 | | 2.00 | | |
| Butylene glycol | 5.00 | | 6.00 | | | | |
| dicaprylate/dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Shea butter | | 2.00 | | | | | |
| PVP hexadecene copolymer | 0.20 | | | 0.50 | | 1.00 | |
| Glycerol | 3.00 | 7.50 | | 7.50 | 5.00 | | 2.50 |
| Xanthan gum | | | 0.05 | | | | |
| Sodium carbomer | | | | 0.15 | | | |
| Vitamin E acetate | 0.60 | | 0.23 | | 0.70 | 1.00 | |
| Fucogel 1000 | | 3.00 | 10.00 | | | | |
| Glycine soya | | | | 0.50 | | 1.50 | 1.00 |
| Ethylhexyloxyglycine | 0.30 | | | | | | |
| DMDM hydantoin | | 0.60 | 0.40 | 0.20 | | | |
| Glyacil-L | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Iminosuccinic acid | | | | 0.25 | 1.00 | | |
| Ethanol | 2.00 | 1.50 | | 3.00 | | 1.20 | 5.00 |
| Perfume | 0.10 | 0.25 | 0.30 | | 0.40 | 0.20 | |
| Water | ad 100 | ad 100 | ad 100 | ad 100 | ad 100 | ad 100 | ad 100 |

Analogous formulations are also prepared with Polymers 1 to 54 or the quaternized derivatives 2(Q) to 54(Q).

Hydrodispersions

| | 1 | 2 | 3 | 4 | 5 |
|---|---|---|---|---|---|
| Ceteareth-20 | 1.00 | | | 0.50 | |
| Cetyl alcohol | | | 1.00 | | |
| Polymer 1(Q) | 1.90 | 1.6 | 1.90 | 0.55 | 0.7 |
| Aminobenzophenone | 2.00 | 1.50 | 0.75 | 1.00 | 2.10 |
| UVASorb K2A | | 3.50 | | | |
| Ethylhexyl methoxycinnamate | | | | | 5.00 |
| Bis-ethylhexyloxyphenol methoxyphenyl | | 1.50 | | 2.00 | 2.50 |
| triazine | | | | | |
| Butyl methoxydibenzoylmethane | | | 2.00 | | 2.00 |
| Disodium phenyl dibenzimidazole | 2.50 | | 0.50 | 2.00 | |
| tetrasulfonate | | | | | |
| Ethylhexyl triazone | 4.00 | | 3.00 | 4.00 | |
| Octocrylene | | 4.00 | | | |
| Diethylhexyl butamido triazone | 1.00 | | | 2.00 | 1.00 |
| Phenylbenzimidazole sulfonic acid | 0.50 | | 3.00 | | |
| Methylene bis-benzotriazolyl | 2.00 | | 0.50 | 1.50 | 2.50 |
| tetramethylbutylphenol | | | | | |
| Ethylhexyl salicylate | | | 3.00 | | |
| Drometrizole trisiloxane | | | 0.50 | | |
| Terephthalidene dicamphor sulfonic acid | | 1.50 | | 1.00 | 1.00 |
| Diethylhexyl 2,6-naphthalate | | | 7.00 | | 9.00 |
| Titanium dioxide - microfine | 1.00 | | 3.00 | | 3.50 |
| Zinc oxide - microfine | | | | 0.25 | |
| C12-15 alkyl benzoate | 2.00 | 2.50 | | | |
| Dicapryl ether | | 4.00 | | | |
| Butylene glycol dicaprylate/dicaprate | 4.00 | | 2.00 | 6.00 | |
| Dicapryl carbonate | | 2.00 | 6.00 | | |
| Dimethicone | | 0.50 | 1.00 | | |
| Phenyltrimethicone | 2.00 | | 0.50 | | |
| Shea butter | | 2.00 | | 5.00 | |
| PVP hexadecene copolymer | 0.50 | | | 0.50 | 1.00 |
| Tricontanyl PVP | 0.50 | | 1.00 | | |
| Ethylhexylglycerol | | | 1.00 | | 0.80 |
| Glycerol | 3.00 | 7.50 | | 7.50 | 8.50 |
| Glycine soya | | | 1.50 | | 1.00 |
| Vitamin E acetate | 0.50 | | 0.25 | | 1.00 |
| Alpha glucosyl rutin | 0.60 | | | 0.25 | |
| Fucogel 1000 | | 2.50 | 0.50 | | 2.00 |
| DMDM hydantoin | | 0.60 | 0.45 | 0.25 | |
| Glyacil-S | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume | 0.20 | | 0.05 | 0.40 | |
| Water | ad 100 | ad 100 | ad 100 | ad 100 | ad 100 |

Analogous formulations are also prepared with Polymers 1 to 54 or the quaternized derivatives 2(Q) to 54(Q).

W/O sunscreen formulations

| | 1 | 2 | 3 | 4 | 5 |
|---|---|---|---|---|---|
| Cetyldimethicone copolyol | | 2.50 | | 4.00 | |
| Polyglyceryl-2 dipolyhydroxystearate | 5.00 | | | | 4.50 |
| PEG-30 dipolyhydroxystearate | | | 5.00 | | |
| Polymer 1(Q) | 2.00 | 0.90 | 0.90 | 1.4 | 1.2 |
| Aminobenzophenone | 2.00 | 1.50 | 0.75 | 1.00 | 2.10 |
| UVASorb K2A | | 2.00 | | | |
| Ethylhexyl methoxycinnamate | | | | | 5.00 |
| Bis-ethylhexyloxyphenol methoxyphenyl triazine | | 1.50 | | 2.00 | 2.50 |
| Butyl methoxydibenzoylmethane | | | 2.00 | | 2.00 |
| Disodium phenyl dibenzimidazole tetrasulfonate | 2.50 | | 0.50 | 2.00 | |
| Ethylhexyl triazone | 4.00 | | 3.00 | 4.00 | |
| Octocrylene | | 4.00 | | | |
| Diethylhexyl butamido triazone | 1.00 | | | 2.00 | 1.00 |
| Phenylbenzimidazole sulfonic acid | 0.50 | | 3.00 | | |
| Methylene bis-benzotriazolyl | 2.00 | | 0.50 | 1.50 | 2.50 |
| tetramethylbutylphenol | | | | | |
| Ethylhexyl salicylate | | | 3.00 | | |
| Drometrizole trisiloxane | | | 0.50 | | |
| Terephthalidene dicamphor sulfonic acid | | 1.50 | | 1.00 | 1.00 |
| Diethylhexyl 2,6-napthalate | | | 7.00 | | 9.00 |
| Titanium dioxide - microfine | 1.00 | | 3.00 | | 3.50 |
| Zinc oxide - microfine | | | | 0.25 | |
| Mineral oil | | 12.00 | 10.00 | | 8.00 |
| C12-15 alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone | | 4.00 | 1.00 | 5.00 | |
| Cyclomethicone | 2.00 | 25.00 | | | 2.00 |
| Shea butter | | | 3.00 | | |
| Petrolatum | | 4.50 | | | |
| PVP hexadecene copolymer | 0.50 | | | 0.50 | 1.00 |
| Ethylhexylglycerol | | 0.30 | 1.00 | | 0.50 |
| Glycerol | 3.00 | 7.50 | | 7.50 | 8.50 |
| Glycine soya | | 1.00 | 1.50 | | 1.00 |
| MgSO₄ | 1.00 | 0.50 | | 0.50 | |
| MgCl₂ | | | 1.00 | | 0.70 |
| Vitamin E acetate | 0.50 | | 0.25 | | 1.00 |
| Ascorbyl palmitate | 0.50 | | | 2.00 | |
| Fucogel 1000 | | | | 3.50 | 7.00 |
| DMDM hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume | 0.20 | | 0.40 | 0.35 | |
| Water | ad 100 | ad 100 | ad 100 | ad 100 | ad 100 |

Analogous formulations are also prepared with Polymers 1 to 54 or the quaternized derivatives 2(Q) to 54(Q).

Solid-stabilized emulsions

| | 1 | 2 | 3 | 4 | 5 |
|---|---|---|---|---|---|
| Mineral oil | | | 16.00 | 16.00 | |
| Octyldodecanol | 9.00 | 9.00 | 5.00 | | |
| Caprylic/capric triglyceride | 9.00 | 9.00 | 6.00 | | |
| C12-15 alkyl benzoate | | | | 5.00 | 8.00 |
| Butylene glycol dicaprylate/dicaprate | | | | | 8.00 |
| Dicaprylyl ether | 9.00 | | | 4.00 | |
| Dicaprylyl carbonate | | 9.00 | | | |
| Hydroxyoctacosanyl hydroxystearate | 2.00 | 2.00 | 2.20 | 2.50 | 1.50 |
| Disteardimonium hectorite | 1.00 | 0.75 | | 0.50 | 0.25 |
| Microcrystalline wax + liquid paraffin | | 0.35 | | | 5.00 |
| Hydroxypropyl methyl cellulose | | | 0.10 | | 0.05 |
| Dimethicone | | | | | 3.00 |
| Polymer 1(Q) | 2.95 | 3.20 | 2.10 | 1.80 | 3.50 |
| Aminobenzophenone | 2.00 | 1.50 | 0.75 | 1.00 | 2.10 |
| UVASorb K2A | | | 3.00 | | |
| Ethylhexyl methoxycinnamate | | | | | 5.00 |
| Bis-ethylhexyloxyphenol methoxyphenyl triazine | | 1.50 | | 2.00 | 2.50 |
| Butyl methoxydibenzoylmethane | | | 2.00 | | 2.00 |
| Disodium phenyl dibenzimidazole tetrasulfonate | 2.50 | | 0.50 | 2.00 | |
| Ethylhexyl triazone | 4.00 | | 3.00 | 4.00 | |
| Octocrylene | | 4.00 | | | |
| Diethylhexyl butamido triazone | 1.00 | | | 2.00 | 1.00 |
| Phenylbenzimidazole sulfonic acid | 0.50 | | 3.00 | | |
| Methylene bis-benzotriazolyl | 2.00 | | 0.50 | 1.50 | 2.50 |
| tetramethylbutylphenol | | | | | |
| Ethylhexyl salicylate | | | 3.00 | | |
| Drometrizole trisiloxane | | | 0.50 | | |
| Terephthalidene dicamphor sulfonic acid | | 1.50 | | 1.00 | 1.00 |
| Diethylhexyl 2,6-naphthalate | | | 7.00 | | 8.50 |
| Titanium dioxide - microfine | 1.00 | | 3.00 | | 3.50 |
| Zinc oxide - microfine | | | | 0.25 | |
| Titanium dioxide + alumina + simethicone + water | | 2.00 | 4.00 | 2.00 | 4.00 |
| Titanium dioxide + trimethoxycaprylylsilane | 2.50 | | | 6.00 | 2.50 |
| Silica dimethyl silylate | | | 1.00 | | |
| Boron nitride | 2.00 | | | | |
| Starch/sodium metaphosphate polymer | | 0.50 | | | |
| Tapioca starch | 5.00 | 7.00 | 8.50 | 3.00 | 4.50 |
| Sodium chloride | | | | 1.00 | |
| Glycerol | 1.00 | 1.00 | 1.00 | 1.00 | 1.00 |
| Trisodium EDTA | 5.00 | 10.00 | 3.00 | 6.00 | 10.00 |
| Vitamin E acetate | 1.00 | 1.00 | | 1.00 | |
| Ascorbyl palmitate | | 0.60 | | | 0.20 |
| Methylparaben | | | | | 0.20 |
| Propylparaben | | | 0.20 | | |
| Phenoxyethanol | | | 0.40 | 0.50 | 0.40 |
| Hexamidine diisethionate | | | | | 0.08 |
| Diazolidinyl urea | | | 0.23 | 0.20 | |
| Ethanol | 5.00 | | 3.00 | 4.00 | |
| Perfume | 0.20 | | 0.30 | 0.10 | |
| Water | ad 100 | ad 100 | ad 100 | ad 100 | ad 100 |

Analogous formulations are also prepared with Polymers 1 to 54 or the quaternized derivatives 2(Q) to 54(Q).

Sticks

| | 1 | 2 | 3 | 4 |
|---|---|---|---|---|
| Caprylic/capric triglyceride | 12.00 | 10.00 | 6.00 | |
| Octyldodecanol | 7.00 | 14.00 | 8.00 | 3.00 |
| Butylene glycol dicaprylate/dicaprate | | | | 12.00 |
| Pentaerythrityl tetraisostearate | 10.00 | 6.00 | 8.00 | 7.00 |
| Polyglyceryl-3 diisostearate | 2.50 | | | |
| Bis-diglyceryl polyacyladipate-2 | 9.00 | 8.00 | 10.00 | 8.00 |
| Cetearyl alcohol | 8.00 | 11.00 | 9.00 | 7.00 |
| Myristyl myristate | 3.50 | 3.00 | 4.00 | 3.00 |
| Beeswax | 5.00 | 5.00 | 6.00 | 6.00 |
| Carnauba wax | 1.50 | 2.00 | 2.00 | 1.50 |
| Alba wax | 0.50 | 0.50 | 0.50 | 0.40 |
| C16-40 alkyl stearate | | 1.50 | 1.50 | 1.50 |
| Polymer 1(Q) | 2.00 | 3.00 | 1.60 | 0.95 |
| Aminobenzophenone | 2.00 | 1.50 | 0.75 | 9.00 |
| UVASorb K2A | | 2.00 | | 4.00 |
| Ethylhexyl methoxycinnamate | | 3.00 | | |
| Bis-ethylhexyloxyphenol methoxyphenyl triazine | | 1.50 | | 2.00 |
| Butyl methoxydibenzoylmethane | | | 2.00 | |
| Disodium phenyl dibenzimidazole tetrasulfonate | 2.50 | | 0.50 | 2.00 |
| Ethylhexyl triazone | 4.00 | | 3.00 | 4.00 |
| Octocrylene | | 4.00 | | |
| Diethylhexyl butamido triazone | 1.00 | | | 2.00 |
| Phenylbenzimidazole sulfonic acid | 0.50 | | 3.00 | |
| Methylene bis-benzotriazolyl | 2.00 | | 0.50 | 1.50 |
| tetramethylbutylphenol | | | | |
| Ethylhexyl salicylate | | | 3.00 | |
| Drometrizole trisiloxane | | | 0.50 | |
| Terephthalidene dicamphor sulfonic acid | | 1.50 | | 1.00 |
| Diethylhexyl 2,6-naphthalate | | | 7.00 | |
| Titanium dioxide - microfine | 1.00 | | 3.00 | |
| Zinc oxide - microfine | | | | 0.25 |
| Vitamin E acetate | 0.50 | 1.00 | | |
| Ascorbyl palmitate | 0.05 | | 0.05 | |
| Buxux chinensis | 2.00 | 1.00 | | 1.00 |
| Perfume, BHT | 0.10 | 0.25 | | 0.35 |
| Ricinus communis | ad 100 | ad 100 | ad 100 | ad 100 |

Analogous formulations are also prepared with Polymers 1 to 54 or the quaternized derivatives 2(Q) to 54(Q).

PIT emulsions

| | 1 | 2 | 3 | 4 | 5 | 6 | 7 | 8 |
|---|---|---|---|---|---|---|---|---|
| Glycerol monostearate SE | 0.50 | 2.00 | 3.00 | 5.00 | | 0.50 | 4.00 | |
| Glyceryl isostearate | | | | | 3.50 | 4.00 | 2.00 | |
| Isoceteth-20 | | 0.50 | 5.0 | | 2.00 | | | |
| Ceteareth-12 | 4.4 | 5.00 | 4.0 | 1.00 | | 5.8 | 3.50 | 5.00 |
| Ceteareth-20 | 4.6 | 5.00 | | 1.00 | 3.5 | 6.0 | | 3.50 |
| PEG-100 stearate | | | | 2.80 | | 2.30 | 3.30 | |
| Cetyl alcohol | 5.20 | | 1.20 | 1.00 | 1.30 | | 0.50 | 0.30 |
| Cetyl palmitate | 2.50 | 1.20 | | 1.50 | | 0.50 | | 1.50 |
| Cetyl dimethicone copolyol | | | | 0.50 | | 1.00 | | |
| Polyglyceryl-2 | | | | 0.75 | 0.30 | | | |
| Polymer 1(Q) | 2.00 | 1.60 | 0.93 | 1.40 | 0.40 | 2.20 | 0.50 | 0.91 |
| Aminobenzophenone | 2.00 | 1.50 | 0.75 | 1.00 | 2.10 | 4.50 | 5.00 | 2.10 |
| UVASorb K2A | | | 4.00 | | | | 1.50 | |
| Ethylhexyl methoxycinnamate | | | | | 5.00 | 6.00 | 8.00 | 5.00 |
| Bis-ethylhexyloxyphenol | | 1.50 | | 2.00 | 2.50 | | 2.50 | 2.50 |
| methoxyphenyl triazine | | | | | | | | |
| Butyl methoxydibenzoylmethane | | | 2.00 | | 2.00 | 1.50 | | 2.00 |
| Disodium phenyl dibenzimidazole | 2.50 | | 0.50 | 2.00 | | 0.30 | | |
| tetrasulfonate | | | | | | | | |
| Ethylhexyl triazone | 4.00 | | 3.00 | 4.00 | | 2.00 | | |
| Octocrylene | | 4.00 | | | | | 7.50 | |
| Diethylhexyl butamido triazone | 1.00 | | | 2.00 | 1.00 | | 1.00 | 1.00 |
| Phenylbenzimidazole sulfonic acid | 0.50 | | 3.00 | | | | | |
| Methylene bis-benzotriazolyl | 2.00 | | 0.50 | 1.50 | 2.50 | | | 2.50 |
| tetramethylbutylphenol | | | | | | | | |
| Ethylhexyl salicylate | | | 3.00 | | | | 5.00 | |
| Drometrizole trisiloxane | | | 0.50 | | | 1.00 | | |
| Terephthalidene dicamphor sulfonic | | 1.50 | | 1.00 | 1.00 | | 0.50 | 1.00 |
| acid | | | | | | | | |
| Diethylhexyl 2,6-naphthalate | | | 7.00 | | 10.00 | 7.50 | | 8.00 |
| Titanium dioxide - microfine | 1.00 | | 3.00 | | 3.50 | | 1.50 | 3.50 |
| Zinc oxide - microfine | | | | 0.25 | | 2.00 | | |
| C12-15 alkyl benzoate | 3.50 | | | 6.35 | | | | 0.10 |
| Cocoglycerides | | 3.00 | | 3.00 | | | | 1.00 |
| Dicapryl ether | 4.50 | | | | | | | |
| Dicaprylyl carbonate | | 4.30 | | 3.00 | | | | 7.00 |
| Dibutyl adipate | | | | 0.50 | | | | 0.30 |
| Phenyltrimethicone | 2.00 | | | 3.50 | | 2.00 | | |
| Cyclomethicone | | 3.00 | | | | | | |
| Ethyl galactomannan | | 0.50 | | | 2.00 | | | |
| Hydrogenated cocoglycerides | | | | | 3.00 | 4.00 | | |
| Abil wax 2440 | | | | | | 1.50 | 2.00 | |
| PVP hexadecene copolymer | | | | 1.00 | 1.20 | | | |
| Glycerol | 4.00 | 6.00 | 5.00 | | 8.00 | 10.00 | | |
| Vitamin E acetate | 0.20 | 0.30 | 0.40 | | 0.30 | | | |
| Shea butter | | 2.00 | | 3.60 | | 2.00 | | |
| Iodopropyl butylcarbamate | 0.12 | | | | 0.20 | | | |
| Fucogel 1000 | | | | 0.10 | | | | |
| DMDM hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Octyloxyglycerol | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume | 0.20 | | 0.20 | | 0.24 | 0.16 | 0.10 | 0.10 |
| Water | ad 100 | ad 100 | ad 100 | ad 100 | ad 100 | ad 100 | ad 100 | ad 100 |

Analogous formulations are also prepared with Polymers 1 to 54 or the quaternized derivatives 2(Q) to 54(Q).

Cosmetic after-sun formulations:

| | 1 | 2 | 3 | 4 | 5 |
|---|---|---|---|---|---|
| Ceteareth-20 | 1.00 | | | 0.50 | |
| Cetyl alcohol | | | 1.00 | | |
| Luvigel EM | | | 0.9 | | |
| Acrylate/C10-30 alkyl acrylate crosspolymer | | | | 0.10 | |
| Xanthan gum | | 0.30 | | | |
| Polymer 1(Q) | 0.90 | 1.00 | 0.8 | 1.7 | 2.1 |
| C12-15 alkyl benzoate | 2.00 | 2.50 | | | |
| Dicapryl ether | | 4.00 | | | |
| Butylene glycol dicaprylate/dicaprate | 4.00 | | 2.00 | 6.00 | |
| Dicapryl carbonate | | 2.00 | 6.00 | | |
| Dimethicone | | 0.50 | 1.00 | | |
| Phenyltrimethicone | 2.00 | | 0.50 | | |
| Tricontanyl PVP | 0.50 | | 1.00 | | |
| Ethylhexylglycerol | | | 1.00 | | 0.80 |
| Glycerol | 3.00 | 7.50 | | 7.50 | 8.50 |
| Glycine soya | | | 1.50 | | 1.00 |
| Vitamin E acetate | 0.50 | | 0.25 | | 1.00 |
| Alpha glucosyl rutin | 0.60 | | | 0.25 | |
| Trisodium EDTA | | 0.01 | 0.05 | | 0.10 |
| Ethanol | 15.00 | 10.00 | 8.00 | 12.00 | 9.00 |
| Perfume | 0.20 | | 0.05 | 0.40 | |
| Water | ad 100 | ad 100 | ad 100 | ad 100 | ad 100 |

Analogous formulations are also prepared with Polymers 1 to 54 or the quaternized derivatives 2(Q) to 54(Q).

Decorative cosmetic formulations:

| | 1 | 2 | 3 | 4 | 5 | 6 | 7 |
|---|---|---|---|---|---|---|---|
| Glycerol monostearate SE | 0.50 | 1.00 | 3.00 | | | 1.50 | |
| Glyceryl stearate citrate | 2.00 | | 1.00 | 2.00 | 4.00 | | |
| Stearic acid | | 3.00 | | 2.00 | | | |
| PEG-40 stearate | 0.50 | | | | | 2.00 | |
| Cetyl phosphate | | | | | | 1.00 | |
| Cetearyl sulfate | | | | | | | 0.75 |
| Stearyl alcohol | | | 3.00 | | | 2.00 | 0.60 |
| Cetyl alcohol | 2.50 | 1.10 | | 1.50 | 0.60 | | 2.00 |
| Polymer 1(Q) | 2.00 | 1.3 | 0.8 | 2.3 | 1.4 | 1.3 | 1.00 |
| Titanium dioxide | 10.00 | 12.00 | 9.00 | 8.50 | 11.00 | 9.50 | 10.00 |
| Iron oxides | 2.00 | 4.00 | 3.00 | 5.00 | 3.40 | 6.00 | 4.40 |
| Zinc oxide | | 4.00 | | 2.00 | | 3.00 | |
| C12-15 alkyl benzoate | | 0.25 | | | 4.00 | 7.00 | |
| Dicapryl ether | | | 3.50 | | 2.00 | | |
| Butylene glycol | 5.00 | | 6.00 | | | | |
| dicaprylate/dicaprate | | | | | | | |
| Cococglycerides | | | 6.00 | | 2.00 | | |
| Dimethicone | 0.50 | | 1.00 | | 2.00 | | |
| Cyclomethicone | 2.00 | | 0.50 | | 0.50 | | |
| Shea butter | | 2.00 | | | | | |
| PVP hexadecene copolymer | 0.20 | | | 0.50 | | 1.00 | |
| Glycerol | 3.00 | 7.50 | | 7.50 | 5.00 | | 2.50 |
| Xanthan gum | 0.15 | | | | | | |
| Vitamin E acetate | 0.60 | | 0.23 | | 0.70 | 1.00 | |
| Glycine soya | | | | 0.50 | | 1.50 | 1.00 |
| Ethylhexyloxyglycine | 0.30 | | | | | | |
| DMDM hydantoin | | 0.60 | 0.40 | 0.20 | | | |
| Glyacil-L | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Iminosuccinic acid | | | | 0.25 | 1.00 | | |
| Ethanol | 2.00 | 1.50 | | 3.00 | | 1.20 | 5.00 |
| Perfume | 0.10 | 0.25 | 0.30 | | 0.40 | 0.20 | |
| Water | ad 100 | ad 100 | ad 100 | ad 100 | ad 100 | ad 100 | ad 100 |

Analogous formulations are also prepared with Polymers 1 to 54 or the quaternized derivatives 2(Q) to 54(Q).

Peeling cream, O/W type
Phase A

| | |
|---|---|
| 3.00 | Ceteareth-6 |
| 1.50 | Ceteareth-25 |
| 3.00 | glyceryl stearate |
| 5.00 | cetearyl alcohol, sodium cetearyl sulfate |
| 6.00 | cetearyl octanoate |
| 6.00 | mineral oil |
| 0.20 | bisabolol |

Phase B

| | |
|---|---|
| 2.00 | propylene glycol |
| 0.10 | disodium EDTA |
| 1.50 | Polymer 1(Q) |
| q.s. | preservative |
| 59.70 | dist. water |

Phase C

| | |
|---|---|
| 0.50 | tocopheryl acetate |
| q.s. | perfume oil |

Phase D

| | |
|---|---|
| 10.00 | polyethylene |

Preparation:

Phases A and B are heated separately to approx. 80° C. Phase B is stirred into Phase A and the mixture is homogenized and cooled to approx. 40° C. Phase C is added and the mixture is briefly homogenized again. Phase D is then stirred in from the bottom.

Analogous formulations are also prepared with Polymers 1 to 54 or the quaternized derivatives 2(Q) to 54(Q).

Lip care cream
Phase A

| | |
|---|---|
| 10.00 | cetearyl octanoate |
| 5.00 | polybutene |
| 1.40 | Polymer 1(Q) |

Phase C

| | |
|---|---|
| 2.00 | Ceteareth-6 |
| 2.00 | Ceteareth-25 |
| 2.00 | glyceryl stearate |
| 2.00 | cetyl alcohol |
| 1.00 | Dimethicone |
| 1.00 | 3-benzophenone |
| 0.20 | bisabolol |
| 6.00 | mineral oil |

Phase D

| | |
|---|---|
| 3.00 | panthenol |
| 3.00 | propylene glycol |
| q.s. | preservative |
| 54.00 | dist. water |

Phase E

| | |
|---|---|
| 0.10 | triethanolamine |

Phase F

| | |
|---|---|
| 0.50 | tocopheryl acetate |
| 0.10 | tocopherol |
| q.s. | perfume oil |

Preparation:

Phase A is dissolved and homogenized. Phase C is added and the mixture is melted at 80° C. Phase D is heated to 80° C. and added to Phase ABC and the mixture is homogenized and cooled to approx. 40° C. Phase E and Phase F are added and the mixture is homogenized again.

Analogous formulations are also prepared with Polymers 1 to 54 or the quaternized derivatives 2(Q) to 54(Q).

Cosmetic O/W formulations:

| | 1 | 2 | 3 | 4 | 5 | 6 | 7 |
|---|---|---|---|---|---|---|---|
| Glycerol monostearate SE | 0.50 | 1.00 | 3.00 | | | 1.50 | |
| Glyceryl stearate citrate | 2.00 | | 1.00 | 2.00 | 4.00 | | |
| Stearic acid | | 3.00 | | 2.00 | | | |
| PEG-40 stearate | 0.50 | | | | | 2.00 | |
| Cetyl phosphate | | | | | | 1.00 | |
| Cetearyl sulfate | | | | | | | 0.75 |
| Stearyl alcohol | | | 3.00 | | | 2.00 | 0.60 |
| Cetyl alcohol | 2.50 | 1.10 | | 1.50 | 0.60 | | 2.00 |
| Polymer 1(Q) | 2.00 | 2.20 | 1.90 | 2.40 | 1.40 | 1.97 | 1.00 |
| Dihydroxyacetone | | | 3.00 | 5.00 | | 4 | |
| Titanium dioxide - microfine | 1.00 | | | | 1.50 | | 1.50 |
| Zinc oxide - microfine | | | | 0.25 | | 2.00 | |
| C12-15 alkyl benzoate | | 0.25 | | | 4.00 | 7.00 | |
| Dicapryl ether | | | 3.50 | | 2.00 | | |
| Butylene glycol | 5.00 | | 6.00 | | | | |
| dicaprylate/dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Shea butter | | 2.00 | | | | | |
| PVP hexadecene copolymer | 0.20 | | | 0.50 | | 1.00 | |
| Glycerol | 3.00 | 7.50 | | 7.50 | 5.00 | | 2.50 |
| Xanthan gum | | | | | | 0.30 | |
| Sodium carbomer | | | | 0.15 | | | |
| Vitamin E acetate | 0.60 | | 0.23 | | 0.70 | 1.00 | |
| Fucogel 1000 | | 3.00 | 10.00 | | | | |
| Glycine soya | | | | 0.50 | | 1.50 | 1.00 |
| Ethylhexyloxyglycine | 0.30 | | | | | | |
| DMDM hydantoin | | 0.60 | 0.40 | 0.20 | | | |
| Glyacil-L | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Iminosuccinic acid | | | | 0.25 | 1.00 | | |
| Ethanol | 2.00 | 1.50 | | 3.00 | | 1.20 | 5.00 |
| Perfume | 0.10 | 0.25 | 0.30 | | 0.40 | 0.20 | |
| Water | ad 100 | ad 100 | ad 100 | ad 100 | ad 100 | ad 100 | ad 100 |

Analogous formulations are also prepared with Polymers 1 to 54 or the quaternized derivatives 2(Q) to 54(Q).

Cosmetic W/O formulations:

| | 1 | 2 | 3 | 4 | 5 |
|---|---|---|---|---|---|
| Cetyldimethicone copolyol | | 2.50 | | 4.00 | |
| Polyglyceryl-2 dipolyhydroxystearate | 5.00 | | | | 4.50 |
| PEG-30 dipolyhydroxystearate | | | 5.00 | | |
| Polymer 1(Q) | 2.00 | 1.70 | 3.00 | 2.50 | 4.00 |
| Titanium dioxide - microfine | 1.00 | | 0.90 | | 0.50 |
| Zinc oxide - microfine | | 0.90 | | 0.25 | |
| Mineral oil | | 12.00 | 10.00 | | 8.00 |
| C12-15 alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone | | 4.00 | 1.00 | 5.00 | |
| Cyclomethicone | 2.00 | 25.00 | | | 2.00 |
| Shea butter | | | 3.00 | | |
| Petrolatum | | 4.50 | | | |
| PVP hexadecene copolymer | 0.50 | | | 0.50 | 1.00 |
| Ethylhexylglycerol | | 0.30 | 1.00 | | 0.50 |
| Glycerol | 3.00 | 7.50 | | 7.50 | 8.50 |
| Glycine soya | | 1.00 | 1.50 | | 1.00 |
| MgSO₄ | 1.00 | 0.50 | | 0.50 | |
| MgCl₂ | | | 1.00 | | 0.70 |
| Vitamin E acetate | 0.50 | | 0.25 | | 1.00 |
| Ascorbyl palmitate | 0.50 | | | 2.00 | |
| DMDM hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume | 0.20 | | 0.40 | 0.35 | |
| Water | ad 100 | ad 100 | ad 100 | ad 100 | ad 100 |

Analogous formulations are also prepared with Polymers 1 to 54 or the quaternized derivatives 2(Q) to 54(Q).

1-16. (canceled)

17. A method for modifying the rheology of aqueous compositions, the method including combining an aqueous composition with a water-soluble or water-dispersible polymer obtainable by the polymerization of a mixture comprising:
a) 99.99 to 10% by weight of at least one α,β-ethylenically unsaturated compound having at least one cationogenic and/or cationic group per molecule, wherein a) is selected from
aiii) N,N-diallylamines,
aiv) vinyl- and allyl-substituted nitrogen heterocycles,
av) vinyl- and allyl-substituted heteroaromatic compounds and
avi) mixtures thereof,
b) 0 to 90% by weight of at least one monoethylenically unsaturated compound containing amide groups which differs from a),
c) 0.01 to 5% by weight of a crosslinking agent,
d) 0 to 15% by weight of at least one monoethylenically unsaturated compound d1) comprising at least one group selected from the group comprising optionally substituted C5-C30-alkyl, C5-C30-alkenyl, C5-C8-cycloalkyl, aryl, arylalkyl and heteroaryl, and/or a reactive precursor d2), and
e) 0 to 30% by weight of other monoethylenically unsaturated compounds differing from a) to d),
in the presence of
f) 0 to 70% by weight, based on the sum of components a) to e), of a polyether-containing compound,
with the proviso that the amounts of components a) to e) add up to 100% by weight,
the polymerization being carried out in the presence of less than 69% by weight of cyclohexane and less than 12% by weight of water, based on the total amount of all the components present during the polymerization, and in the absence of supercritical carbon dioxide.

18. The method according to claim 17, wherein a) is selected from aiv) and especially from vinylimidazoles of general formula (III): 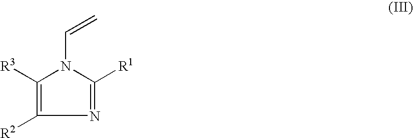
in which R¹ to R³ are hydrogen, C₁-C₄-alkyl or phenyl.

19. The method according to claim 18, wherein R¹ to R³ are hydrogen.

20. The method according to claim 17, wherein at least one other component b) is selected from α,β-ethylenically unsaturated compounds containing amide groups of general formula IV: 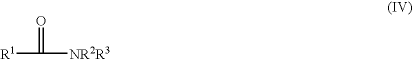
in which
R¹ is a group of the formula CH₂═CR⁴—, where R⁴═H or C₁-C₄-alkyl, and R² and R³ independently of one another are H, alkyl, cycloalkyl, heterocycloalkyl, aryl or heteroaryl, or R² and R³, together with the nitrogen atom to which they are bonded, are a five-membered to eight-membered nitrogen heterocycle, or
R² is a group of the formula CH₂═CR⁴— and R¹ and R³ independently of one another are H, alkyl, cycloalkyl, heterocycloalkyl, aryl or heteroaryl, or R¹ and R³, together with the amide group to which they are bonded, are a lactam having 5 to 8 ring atoms.

21. The method according to claim 20, wherein, in formula IV, R² is CH₂═CH— and R¹ and R³, together with the amide group to which they are bonded, are a lactam having 5 ring atoms.

22. The method according to claim 17, wherein the polyether-containing components f) used are compounds of general formula Va or Vb: 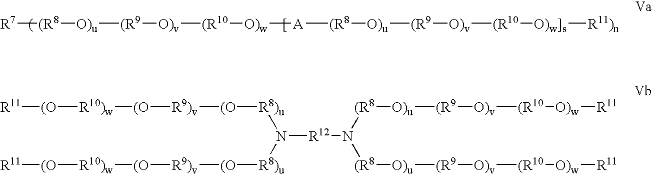
in which:
R⁷ is hydroxyl, amino, C₁-C₂₄-alkoxy, R¹³—COO—, R¹³—NH—COO— or a polyalcohol radical,
R⁸, R⁹ and R¹⁰ independently of one another are —(CH₂)₂—, —(CH₂)₃—, —(CH₂)₄—, —CH₂—CH(CH₃)—, —CH₂—CH(CH₂—CH₃)— or —CH₂—CHOR¹⁴—CH₂—,
R¹¹ is hydrogen, amino-C₁-C₆-alkyl, C₁-C₂₄-alkyl, R¹³—C(═O)— or R¹³—NH—C(═O)—,
R¹² is a C₁-C₂₀-alkylene group whose carbon chain can be interrupted by 1 to 10 non-adjacent oxygen atoms,
R¹³ is C₁-C₂₄-alkyl,
R¹⁴ is hydrogen, C₁-C₂₄-alkyl or R¹³—CO—,
A is —C(═O)—O—, —C(═O)—B—C(═O)—O— or —C(═O)—NH—B—NH—C(═O)—O—,
B is —(CH₂)ₜ—, optionally substituted cycloalkylene, optionally substituted heterocycloalkylene or optionally substituted arylene,
n is 1 or, if R⁷ is a polyalcohol radical, 1 to 8,
s is 0 to 500,
t is 1 to 12,
u independently of one another are each 1 to 5000,
v independently of one another are each 0 to 5000, and
w independently of one another are each 0 to 5000.

23. The method according to claim 22, wherein the polyether-containing components f) used are homopolymers and copolymers of ethylene oxide and/or propylene oxide having a weight-average molecular weight M_{w} ranging from 1000 to 100,000 g/mol, which can be capped with end groups at one or both ends.

24. The method according to claim 17, wherein the polyether-containing component f) is present in the polymerization in an amount ranging from 5 to 70% by weight, based on the total amount of components a) to e).

25. The method according to claim 17, wherein the crosslinking agent c) is selected from the group comprising pentaerythrityl triallyl ether, methylenebisacrylamide, N,N′-divinylethyleneurea, triallylamine and triallylmonoalkylammonium salts, and acrylic acid esters of ethylene glycol, butanediol, trimethylolpropane or glycerol, or acrylic acid esters of glycol, butanediol, trimethylolpropane or glycerol which has been reacted with ethylene oxide and/or epichlorohydrin.

26. The method according to claim 17, wherein the mixture to be polymerized comprises 0.05 to 2% by weight of the crosslinking agent c).

27. The method according to claim 28, wherein the compound d) is selected from the group comprising C₁₈-C₃₀-alkyl(meth)acrylates and C₁₈-C₃₀-alkyl vinyl ethers.

28. The method according to claim 17, wherein the polymer is partially or completely protonated, or quaternized to an extent ranging from 20 to 99 mol %, after or during the polymerization, if the component a) used for the polymerization is a monomer that is only partially protonated or quaternized, if at all.

29. A polymer as defined in claim 17.

30. A cosmetic or pharmaceutical product comprising at least one polymer according to claim 29.

31. A cosmetic or pharmaceutical product comprising at least one polymer according to claim 29 in an amount ranging from 0.01 to 5% by weight.

32. The method according to claim 18, wherein at least one other component b) is selected from α,β-ethylenically unsaturated compounds containing amide groups of general formula IV: 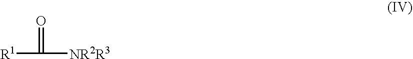
in which
R¹ is a group of the formula CH₂═CR⁴—, where R⁴═H or C₁-C₄-alkyl, and R² and R³ independently of one another are H, alkyl, cycloalkyl, heterocycloalkyl, aryl or heteroaryl, or R² and R³, together with the nitrogen atom to which they are bonded, are a five-membered to eight-membered nitrogen heterocycle, or
R² is a group of the formula CH₂═CR⁴— and R¹ and R³ independently of one another are H, alkyl, cycloalkyl, heterocycloalkyl, aryl or heteroaryl, or R¹ and R³, together with the amide group to which they are bonded, are a lactam having 5 to 8 ring atoms.

33. The method according to claim 18, wherein the polyether-containing components f) used are compounds of general formula Va or Vb: 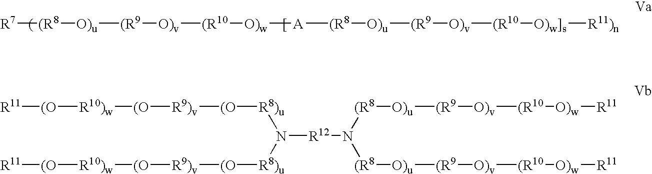
in which:
R⁷ is hydroxyl, amino, C₁-C₂₄-alkoxy, R¹³—COO—, R¹³—NH—COO— or a polyalcohol radical,
R⁸, R⁹ and R¹⁰ independently of one another are —(CH₂)₂—, —(CH₂)₃—, —(CH₂)₄—, —CH₂—CH(CH₃)—, —CH₂—CH(CH₂—CH₃)— or —CH₂—CHOR¹⁴—CH₂—,
R¹¹ is hydrogen, amino-C₁-C₆-alkyl, C₁-C₂₄-alkyl, R¹³—C(═O)— or R¹³—NH—C(═O)—,
R¹² is a C₁-C₂₀-alkylene group whose carbon chain can be interrupted by 1 to 10 non-adjacent oxygen atoms,
R¹³ is C₁-C₂₄-alkyl,
R¹⁴ is hydrogen, C₁-C₂₄-alkyl or R¹³—CO—,
A is —C(═O)—O—, —C(═O)—B—C(═O)—O— or —C(═O)—NH—B—NH—C(═O)—O—,
B is —(CH₂)ₜ—, optionally substituted cycloalkylene, optionally substituted heterocycloalkylene or optionally substituted arylene,
n is 1 or, if R⁷ is a polyalcohol radical, 1 to 8,
s is 0 to 500,
t is 1 to 12,
u independently of one another are each 1 to 5000,
v independently of one another are each 0 to 5000, and
w independently of one another are each 0 to 5000.

34. The method according to claim 18, wherein the polyether-containing component f) is present in the polymerization in an amount ranging from 5 to 70% by weight, based on the total amount of components a) to e).

35. The method according to claim 18, wherein the crosslinking agent c) is selected from the group comprising pentaerythrityl triallyl ether, methylenebisacrylamide, N,N′-divinylethyleneurea, triallylamine and triallylmonoalkylammonium salts, and acrylic acid esters of ethylene glycol, butanediol, trimethylolpropane or glycerol, or acrylic acid esters of glycol, butanediol, trimethylolpropane or glycerol which has been reacted with ethylene oxide and/or epichlorohydrin.

36. The method according to claim 18, wherein the mixture to be polymerized comprises 0.05 to 2% by weight of the crosslinking agent c).
